(19) United States
(12) Patent Application Publication
(10) Pub. No.: US 2026/0308226 A1
(43) Pub. Date: Oct. 8, 2026

(54) NK CELL COMPOSITIONS AND PREPARATIONS FOR IMMUNOTHERAPY AND METHODS FOR THEIR PRODUCTION

(71) Applicant: Deverra Therapeutics Inc., Seattle, WA (US)

(72) Inventors: Colleen Delaney, Seattle, WA (US); Carrie Stoltzman, Seattle, WA (US); Elizabeth Dam, Seattle, WA (US); Mary Prieve, Seattle, WA (US)

(73) Assignee: Deverra Therpaeutics Inc., Seattle, WA (US)

(21) Appl. No.: 19/708,094

(22) Filed: Jun. 15, 2026

Related U.S. Application Data
(63) Continuation of application No. 17/631,035, filed on Jul. 29, 2020, now Pat. No. 12,655,190.
(60) Provisional application No. 62/880,044, filed on Jul. 29, 2019.
(60) Provisional application No. 62/892,779, filed on Aug. 28, 2019.
(60) Provisional application No. 62/960,507, filed on Jan. 13, 2020.

(51) Int. Cl. C07K 14/705; A61K 40/15; A61K 40/31; A61K 40/42; C07K 16/28; C12N 5/0783

(52) U.S. Cl. CPC C07K 14/7051; A61K 40/15; A61K 40/31; A61K 40/4211; A61K 40/4255; C07K 14/70521; C07K 14/70578; C07K 16/2803; C12N 5/0646; A61K 2239/31; A61K 2239/38; A61K 2239/47; A61K 2239/48; C07K 2317/53; C07K 2317/622; C12N 2501/145; C12N 2501/2302; C12N 2501/2303; C12N 2501/2306; C12N 2501/2315; C12N 2501/26; C12N 2501/42; C12N 2506/03

(57) ABSTRACT

The present invention provides NK cell compositions and/or preparations and methods of using such NK cell compositions and/or preparations for immunotherapy. The NK cell compositions and/or preparations can be used in therapies of a broad range of viral infections, bacterial infections, cancer and leukemia malignancies, and other diseases.

### CROSS-REFERENCES TO RELATED APPLICATIONS

This application is a continuation of U.S. application Ser. No. 17/631,035, filed Jan. 28, 2022, which is a National Stage of International Application No. PCT/US2020/044117, filed Jul. 29, 2020, which claims the benefit of U.S. Provisional Application No. 62/880,044, filed Jul. 29, 2019, U.S. Provisional Application No. 62/892,779, filed Aug. 28, 2019, and U.S. Provisional Application No. 62/960,507, filed Jan. 13, 2020, the disclosures of which are incorporated herein by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 1765-P2US.CON_Seq_List_20260615.xml. The XML file is 90,020 bytes; was created on Jun. 15, 2026; contains no new matter; and is being submitted electronically via Patent Center with the filing of the specification.

### FIELD OF THE INVENTION

The present disclosure provides natural killer (NK) cell compositions and/or preparations, and methods of making and using such NK cell compositions and/or preparations for non-autologous immunotherapy. The NK cell compositions and/or preparations can be used in therapies for a broad range of viral and bacterial infections, cancer, including solid tumors and hematologic malignancies (leukemia, lymphoma), and other diseases.

### BACKGROUND OF THE INVENTION

Natural Killer (NK) cells are components of the innate immune system that exhibit a variety of cytotoxic activities against transformed target cells, such as production of certain cytokines, direct cytotoxicity, and mediation of antibody-dependent cell-mediated cytotoxicity (ADCC). NK cells play a key role in host defense against essentially any virus infected or cancer transformed cells and the clearance of these cells. In addition, NK cells have been proposed as effectors for immunotherapy of various conditions including, for example, clearing transformed cancer cells. Various methods have been described for producing NK cells, such as generation from induced pluripotent stem cells, peripheral blood mononuclear cells, peripheral blood stem or progenitor cells, or umbilical cord blood stem or progenitor cells. For each source, the properties of the NK cells depend on their method of generation. Such methods have traditionally relied on many rounds of ex vivo cell division to produce adequate numbers of NK cells for therapeutic purposes. NK cells resulting from such processes tend to be more mature and/or are more likely to exhibit exhaustion following administration to a patient. NK cell exhaustion can be exhibited by reduced production of, for example, interferon gamma (IFNγ), CD107a, granzyme B and/or perforin, decreased cytolytic activity, and the like. Such NK cells are likely to be less active or persistent when administered in vivo. Therefore, there remains a need for NK cell populations and compositions comprising said NK cell populations for therapeutic purposes which exhibit and retain higher levels of activity and/or persistence after administration to patients, as well as methods for producing such NK cell populations and/or compositions.

### SUMMARY OF THE DISCLOSURE

The present disclosure provides compositions of NK cell preparations and methods of making and using such NK cell compositions and/or preparations for immunotherapy. The NK cell compositions and/or preparations can be used in therapies for a broad range of viral infections, malignancies, and other diseases. The NK cell compositions and/or preparations can be comprised of CD56⁺ (mainly NK) cells derived from multiple human donors, with typically at least two or at least four human donors, or from fully matched or partially matched (mismatched) donors. The NK cell compositions and/or preparations can be prepared from pooled, non-HLA matched (unmatched), CD34⁺ hematopoietic stem and progenitor cells (HSPCs), from immunologically matched, or from partially immunologically matched (mismatched) HSPCs by expansion of the HSPCs in vitro or ex vivo, followed by differentiation into CD56⁺ (mainly NK) cells. Significantly, the resulting NK cell compositions and/or preparations whether immunologically unmatched, matched or mismatched, comprise a mixture of CD56⁺ (largely NK) cells and CD56-(non-NK) cells. The CD56⁺ (NK) cells produced by the disclosed methods are highly potent, but less mature than comparable NK cells in the prior art that are typically derived from adult human donors, and thus may exhibit more cytolytic activity and/or persistence in vivo. The non-NK cells present in the herein described compositions and/or preparations are typically of myeloid origin and support NK cell differentiation and activation, eliminating the need for an exogenous feeder cell layer.

In some embodiments, an NK cell composition and/or preparation of the present disclosure comprises from about 50% up to about 80% CD56⁺ cells and about 20% to about 50% CD56-myeloid-derived cells, such as dendritic cells, macrophages, and granulocytes. The CD56⁻ cells produced during the generation of the CD56⁺ are considered endogenous to the CD56⁺ cells. In some embodiments, the NK cell composition and/or preparation comprises from about 50% up to about 85% CD56⁺ cells and about 15% to about 50% CD56-myeloid-derived cells, such as dendritic cells, macrophages, and granulocytes. In some embodiments, the NK cell composition and/or preparation produced by the disclosed methods comprises from about 55% up to about 65% CD56⁺ cells and about 35% to about 45% CD56⁻, myeloid-derived cells, such as dendritic cells, macrophages, and granulocytes. In some embodiments, the NK cell composition and/or preparation produced comprises from about 70% up to about 85% CD56⁺ cells and about 15% to about 30% CD56⁻, myeloid-derived cells, such as dendritic cells, macrophages, and granulocytes.

The above described CD56⁺ subpopulation is mainly comprised of NK cells. In addition to CD56, the CD56⁺ cells can express a high frequency of NKp30, NKp46, NKp44, NKG2A, and/or granzyme B, moderate to a high frequency of perforin and/or CD107a, a low to moderate frequency of NKG2D, and substantially no killer cell immunoglobulin-like receptors (KIRs). In some embodiments the CD56⁺ cells are KIR⁻. (As used herein, “KIR⁻” refers to KIR family members KIR2DL1, KIR2DS1, KIR2DS3, KIR2DS5, KIR2DL2, KIR2DL3, KIR2DS2, KIR2DS4, KIR3DL1, and KIR3DS1.) CD16 (also known as Fc gamma receptor III (FcγRIII)) can be expressed at a low or moderate frequency on NK cells induced to differentiate in in vitro by the methods described herein. In some embodiments, the CD56⁻ cells express a moderate to high frequency of granzyme B, a high frequency of CD107a, and a low frequency of perforin. In some embodiments an expanded NK cell composition and/or preparation comprises cells derived from at least two human donors, without matching to the HLA type of the other donors and without matching to the HLA type of the patient who will receive the NK cells. In other embodiments the expanded NK cell composition and/or preparation comprises cells immunologically matched to the HLA type to the subject. In still other embodiments the expanded NK cell composition and/or preparation comprises cells that are at least partially immunologically matched (mismatched) to the subject.

In some embodiments, in vitro or ex vivo methods for preparing an NK cell composition and/or preparation are provided. The methods typically include selecting a plurality of umbilical cord blood and/or placental blood units without immunological matching to each other; lysing or otherwise depleting red blood cells; depleting the T cells; enriching for CD34⁺ hematopoietic stem and progenitor cells (HSPCs); followed by culturing the CD34⁺ enriched HSPCs in an expansion culture medium and in the absence of feeder cells for a sufficient time to produce an expanded HSPC cell population, wherein the expanded HSPCs do not substantially differentiate into CD56⁺ (mainly NK) cells during the expansion; and then culturing the expanded HSPC cell population in a differentiation culture medium with cytokines and in the absence of feeder cells for a sufficient time to produce an NK cell composition and/or preparation comprising about 50 to about 80% CD56⁺ cells and about 20 to about 50% endogenous CD56⁻ cells. In some embodiments, an NK cell composition and/or preparation comprises from about 50% up to about 85% CD56⁺ cells and about 51% to about 50% CD56⁻, endogenous myeloid-derived cells, such as dendritic cells, macrophages, and granulocytes. In some embodiments, an NK cell composition and/or preparation comprises from about 55% up to about 65% CD56⁺ cells and about 35% to about 45% CD56⁻, endogenous myeloid-derived cells, such as dendritic cells, macrophages, and granulocytes. In some embodiments, an NK cell composition and/or preparation comprises from about 70% up to about 85% CD56⁺ cells and about 15% to about 30% CD56⁻, myeloid-derived cells, such as dendritic cells, macrophages, and granulocytes. The CD56⁺ subpopulation is mainly comprised of NK cells. In addition to CD56, the CD56⁺ cells express a high frequency of NKp30, NKp46, NKp44, NKG2A, and granzyme B, moderate to a high frequency of perforin and CD107a, a low to moderate frequency of NKG2D, and substantially no KIRs. In some embodiments the CD56⁺ cells are KIR⁻. (As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1.) CD16 is expressed at a low or moderate frequency. In some embodiments, the CD56⁻ cells express a moderate to high frequency of granzyme B, a high frequency of CD107a, and a low frequency of perforin.

In other embodiments, the in vitro or ex vivo methods for preparing an NK cell composition and/or preparation comprise selecting fully immunologically matched or mismatched units of umbilical cord blood and/or placental blood units. Immunological matching can be carried out by any method known in the art. Subsequent to selecting the umbilical cord blood and/or placental blood unit(s), the method comprises lysing or otherwise depleting red blood cells; depleting the T cells; enriching for CD34⁺ hematopoietic stem and progenitor cells (HSPCs); followed by culturing the CD34⁺ enriched HSPCs in an expansion culture medium and in the absence of feeder cells for a sufficient time to produce an expanded HSPC cell population, wherein the expanded HSPCs do not substantially differentiate into CD56⁺ (mainly NK) cells during the expansion; and then culturing the expanded HSPC cell population in a differentiation culture medium with cytokines and in the absence of feeder cells for a sufficient time to produce an NK cell composition and/or preparation comprising about 50 to about 80% CD56⁺ cells and about 20 to about 50% endogenous CD56⁻ cells. In some embodiments, the NK cell composition and/or preparation comprises from about 50% up to about 85% CD56⁺ cells and about 15% to about 50% CD56⁻, endogenous myeloid-derived cells, such as dendritic cells, macrophages, and granulocytes. In some embodiments, the NK cell composition and/or preparation comprises from about 55% up to about 65% CD56⁺ cells and about 35% to about 45% CD56⁻, endogenous myeloid-derived cells, such as dendritic cells, macrophages, and granulocytes. In other embodiments, the NK cell composition and/or preparation comprises from about 70% up to about 85% CD56⁺ cells and about 15% to about 30% CD56⁻, endogenous myeloid-derived cells, such as dendritic cells, macrophages, and granulocytes. The CD56⁺ subpopulation is mainly comprised of NK cells. In addition to CD56, the CD56⁺ cells express a high frequency of NKp30, NKp46, NKp44, NKG2A, and granzyme B, moderate to a high frequency of perforin and CD107a, a low to moderate frequency of NKG2D and substantially no KIRs. In some embodiments the CD56⁺ cells are KIR⁻. (As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1.) CD16 can also be expressed at a low or moderate frequency. In some embodiments, the CD56⁻ cells can express a moderate to high frequency of granzyme B, a high frequency of CD107a, and a low frequency of perforin.

In some embodiments, regardless of whether the HSPCs originate from unmatched, matched, or mismatched cord blood units, the HSPC expansion culture medium comprises interleukin-3 (IL-3), interleukin-6 (IL-6), thrombopoietin (TPO), Flt-3 ligand (Flt-3L), stem cell factor (SCF) in a solid phase tissue culture substrate coated with a Notch ligand and fibronectin or fragments thereof. In some embodiments, the only growth factors added to the HSPC in vitro or ex vivo expansion culture medium are IL-3, IL-6, TPO, Flt-3L, and SCF. In some embodiments, the Notch ligand in the HSPC expansion culture medium is Delta1^{ext-IgG}. In some embodiments, the fibronectin or fragments thereof are recombinant human fibronectin or fragments thereof.

Differentiation of the now expanded HSPCs comprises their isolation and in vitro or ex vivo culture of the isolated HSPCs in a cell culture media supplemented with IL-2 and IL-15 to induce differentiation. In some embodiments, the only cytokines added to the in vitro or ex vivo differentiation culture medium are interleukin-2 (IL-2) and interleukin-15 (IL-15). In some embodiments, the amount of IL-2 and IL-15 in the differentiation culture medium is from about 25 U/mL to about 100 U/mL of IL-2 and from about 25 ng/ml to about 50 ng/ml of IL-15. In other embodiments, the amount of IL-2 and IL-15 in the differentiation culture medium can be about 50 U/mL for IL-2 and about 40 ng/mL for IL-15. In some embodiments, the cytokines in the differentiation culture comprise IL-2 and IL-15, wherein other cytokines, such as Flt-3L, fibroblast growth factor 2 (FGF-2), IL-6, IL-7, IL-12, IL-3, GM-CSF, granulocyte-colony stimulating factor (G-CSF), leukemia inhibitory factor (LIF), macrophage inhibitory protein 1 alpha (MIP-1a), SCF, IL-21, IL-18, and 4-1BBL (4-1BB ligand), are not added to the differentiation culture medium. In some embodiments, the differentiation culture medium does not contain added cytokines other than the added IL-2 and IL-15 used to induce differentiation of the NK cells.

In certain embodiments of the disclosure, IL-15 is added during the expansion of the CD34⁺ enriched HSPCs to prime the differentiation of the NK cell compositions and/or preparations. The IL-15 can be added during the last 4 to 7 days of the expansion phase. If the expansion phase is reduced to 7 days, the priming with IL-15 can be during the last about 4 days. Differentiation of the now expanded and primed HSPCs comprises their isolation and in vitro or ex vivo culture of the isolated HSPCs in a cell culture media supplemented with IL-2 and IL-15 to induce differentiation. In some embodiments, the only cytokines added to the in vitro or ex vivo differentiation culture medium are IL-2 and IL-15. In some embodiments, the amount of IL-2 and IL-15 in the differentiation culture medium is from about 25 U/mL to about 100 U/mL of IL-2 and from about 25 ng/mL to about 50 ng/ml of IL-15. In other embodiments, the amount of IL-2 and IL-15 in the differentiation culture medium can be about 50 U/mL for IL-2 and about 40 ng/ml for IL-15. In some embodiments, the cytokines in the differentiation culture comprise IL-2 and IL-15, wherein other cytokines, such as Flt-3L, fibroblast growth factor 2 (FGF-2), IL-6, IL-7, IL-12, IL-3, GM-CSF, granulocyte-colony stimulating factor (G-CSF), leukemia inhibitory factor (LIF), macrophage inhibitory protein 1 alpha (MIP-1a), SCF, IL-21, IL-18, and 4-1BBL (4-1BB ligand), are not added to the differentiation culture medium. In some embodiments, the differentiation culture medium does not contain added cytokines other than the added IL-2 and IL-15 used to induce differentiation of the NK cells.

In certain embodiments of the present methods the non-animal sourced protein used to supplement the differentiation medium is human AB serum, fresh frozen human plasma, or human platelet lysate. Still further, in certain embodiments the HSPCs are not derived from somatic cells embryonic stem cells, peripheral blood mononuclear cells, or induced pluripotent stem cells.

In certain embodiments of the disclosure the methods produce NK cell compositions and/or preparations that comprise less than 2% CD3⁺ cells, less than 2% CD19⁺ cells, and/or less than 2% CD34⁺ cells. The CD56⁺ cells of the compositions and or preparations further express a high frequency of KIR2DL4. In some embodiments the method comprises the use of a Notch ligand that is DXI or an antibody specific for Notch.

In certain embodiments the methods comprise the genetic modification of the NK cell compositions and/or preparations. In certain specific embodiments the genetic modification is done during the expansion phase and in other the genetic modification is done subsequent to differentiation of the NK cells. The cells of the NK cell composition and/or preparation can be genetically modified to express an antigen recognizing receptor. In certain specific embodiments the genetic modification is the introduction of a polynucleotide expressing a T cell receptor (TCR) or a chimeric antigen receptor (CAR). The TCR or the CAR can be designed to specifically bind to a viral antigen, a bacterial antigen, or a tumor specific or tumor associate antigen.

In certain embodiments of the disclosed methods the viral antigen is present in Cytomegalovirus (CMV), Epstein Barr Virus (EBV), Human Immunodeficiency Virus (HIV), Herpes simplex virus (HSV), Hepatitis virus, zika virus, influenza virus, or coronavirus. In specific embodiments, the Herpes virus is HSV 1 or HSV2, the Hepatitis virus is Hepatitis A, B or C, and the coronavirus is SARS-CoV or SARS-CoV-2.

In certain other embodiments the where the CAR is directed to a tumor specific or tumor associated antigen the antigen can be CD19, ROR1, Her2, PSMA, PSCA, mesothelin, or CD20.

The polynucleotide encoding the CAR can comprise an intracellular signaling domain comprising a signaling domain of CD3zeta, CD28, and 4-1BB; at least one co-stimulatory domain comprising a co-stimulatory domain of CD27, CD28, 4-1BB, 2B4, DAP10, DAP12, OX40, CD30, CD40, lymphocyte function-associated antigen-1 (LFA-1), CD2, CD7, LIGHT, NKG2C, or B7-H3; a transmembrane domain comprising a transmembrane domain of CD8, CD28, CD3zeta, CD4, 4-1BB, OX40, ICOS, or NKG2D; and a spacer region comprising a hinge region of IgG₁, the CH₂CH₃ region of an immunoglobulin, a portion of CD3, a portion of CD28, or a portion of CD8. In some embodiments the CAR can comprise a single chain Fv (scFv) having the CDRs of monoclonal antibody FMC63.

In certain embodiments the NK cell compositions and preparations produced by a method disclosed herein can be formulated for infusion into a subject.

In certain embodiments disclosed herein natural killer (NK) cell compositions are produced for use in immunotherapy. The compositions can comprise about 50 to about 80% CD56⁺ cells and about 20 to about 50% endogenous CD56⁻ cells, or about 50 to about 85% CD56⁺ cells and about 15 to about 50% endogenous CD56⁻ cells, wherein the CD56⁺ cells express a high frequency of NKp30, NKp46, NKp44, NKG2A, NKG2D, and granzyme B, a moderate to a high frequency of perforin and CD107a, a low to moderate frequency of CD16 and substantially no KIRs; and wherein the CD56⁻ cells express a moderate to high frequency of granzyme B, a high frequency of CD107a, and a low frequency of perforin, and a pharmaceutically acceptable carrier. In specific embodiments the NK cell composition does not contain exogenous feeder cells.

In certain embodiments, the NK cell composition comprises less than 2% CD3⁺ cells, and/or less than 2% CD19⁺ cells, and/or less than 2% CD34⁺ cells. In specific embodiments the NK cell compositions comprise CD56⁺ cells that further express a high frequency of KIR2DL4.

The NK cell compositions described herein can comprise cells that are genetically modified. The genetically modified cells of the composition can be genetically modified to express an antigen recognizing receptor. These antigen recognizing receptors can be encoded by an introduced polynucleotide expressing a TCR or a CAR. In certain specific embodiments the TCR or the CAR can specifically bind to a viral antigen, a bacterial antigen, or a tumor associated or tumor specific antigen. In more specific embodiments, the viral antigen is present in Cytomegalovirus (CMV), Epstein Barr Virus (EBV), Human Immunodeficiency Virus (HIV), Herpes simplex virus (HSV), Hepatitis virus, zika virus, influenza virus, or coronavirus. In more preferred embodiments the Herpes virus is HSV 1 or HSV 2, the Hepatitis virus is Hepatitis A, B or C, and the coronavirus is SARS-CoV or SARS-CoV-2.

In certain embodiments the NK cell composition and/or preparation comprising a TCR or a CAR, the TCR or CAR is specific for a tumor associated or tumor specific antigen is specific for carbonic anhydrase IX (CAIX), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), epithelial glycoprotein-2 (EGP-2), epithelial glycoprotein-40 (EGP-40), epithelial cell adhesion molecule (EpCAM), receptor tyrosine-protein kinases erb-B2, erb-B3, or erb-B4, folate-binding protein (FBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13Rα2), kappa-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), mesothelin (MSLN), ERBB2, MAGEA3, p53, MART1, GP100, Proteinase3 (PR1), tyrosinase, survivin, hTERT, EphA2, an NKG2D ligand, cancer-testis antigen NY-ESO-1, oncofetal antigen (h5T4), prostate stem cell antigen (PSCA), prostate-specific membrane antigen (PSMA), ROR1, tetraspanin 8 (TSPAN8), tumor-associated glycoprotein 72 (TAG-72), vascular endothelial growth factor R2 (VEGF-R2), Wilms tumor protein (WT-1), BCMA, GPC3, NKCS1, EGF1R, EGFR-VIII, CRLF2, and ERBB. In more specific embodiments, the tumor associated or tumor specific antigen is CD19, ROR1, Her2, PSMA, PSCA, mesothelin, CRLF2, or CD20.

In certain embodiments where the NK cell comprises an antigen recognizing receptor, the antigen recognizing receptor can be a CAR. In more specific embodiments the CAR can comprise an intracellular signaling domain of CD3zeta, CD28, and 4-1BB; at least one co-stimulatory domain of CD27, CD28, 4-1BB, 2B4, DAP10, DAP12, OX40, CD30, CD40, lymphocyte function-associated antigen-1 (LFA-1), CD2, CD7, LIGHT, NKG2C, or B7-H3 costimulatory domain; a transmembrane domain of CD8, CD28, CD3zeta, CD4, 4-1BB, OX40, ICOS, or NKG2D; and a spacer region of IgG₁, the CH₂CH₃ region of an immunoglobulin, a portion of CD3, a portion of CD28, or a portion of CD8. In certain embodiments the

In certain embodiments the NK cell composition can further comprise a cryoprotective agent and the NK cell composition is frozen for future use. In certain specific embodiments the NK cell composition is formulated for infusion into a subject. The formulation can comprise the cryoprotective agent used to store the composition. The NK cell composition can comprise from about 50 million to about 2 billion viable cells. In certain embodiments the NK cell composition comprises from about 50 million to about 2 billion viable CD56⁺ cells.

The present disclosure also provides methods of treating a subject in need thereof, comprising administering a therapeutically effective amount of the NK cell composition described above to the subject. The subject can have for example a cancer expressing a tumor antigen and the NK cells of the composition express an antigen recognizing receptor that binds to the tumor antigen. In other embodiments the subject has a viral or bacterial infection. In certain embodiments, the NK cell composition expresses a chimeric antigen receptor that is specific for a tumor specific or associated antigen, a viral antigen, or a bacterial antigen.

### DESCRIPTION OF THE DRAWINGS

The foregoing aspects and many of the attendant advantages of these methods and compositions and/or preparations will become more readily appreciated as the same become better understood by reference to the following detailed description, when taken in conjunction with the accompanying drawings, wherein:

FIGS. 1A and 1B show the results from 20 batches of NK cell compositions and/or preparations produced following the process described in Example 1. FIG. 1A shows the increase in CD56⁺ cells/starting CD34⁺ cells over the course of a 28-day culture (phases 1 and 2). FIG. 1B demonstrates reproducibility of CD56⁺ cell generation over a 28-day cell culture process.

FIG. 2 shows replacement of fetal bovine serum (FBS) with human AB serum (hABS) or human platelet lysate (hPL) had minimal effects on the cell expansion during differentiation.

FIG. 3 shows replacement of FBS with human AB serum (hABS) or human platelet lysate (hPL) had minimal effects on the percent of CD56⁺ cells produced.

FIG. 4 shows the percent of CD16⁺ cells relative to CD56⁺ NK cells on day 28 of culture comparing different serum supplements used in Phase 2 (the differentiation phase) of the culture.

FIG. 5 shows the percent of NKp46⁺ cells relative to CD56⁺ NK cells on day 28 of culture comparing different serum supplements used in Phase 2 of the culture.

FIG. 6 shows cultures supplemented with hPL developed a more cytotoxic NK cell population than those supplemented with hABS.

FIG. 7 shows cultures supplemented with hPL developed a more cytotoxic NK cell population than those supplemented with hABS.

FIG. 8 shows the NK cell composition and/or preparation (referred to as NK Cell Product) was active against K562 and A549 target cells with similar activity (curves indicated with closed circles and triangles in the middle of the graph) and were more potent than activated adult NK cells against A549 target cells.

FIG. 9 shows the NK cell composition and/or preparation demonstrated cell killing against many of the tested cancer cell lines. For each cell line, the left bar indicates cytotoxicity at 4 hours and the right bar indicates cytotoxicity at 24 hours. The K562 cell line is an accepted standard for NK cell activity and serves as a positive control.

FIG. 10 demonstrates the NK Cell Product was able to reduce tumor burden, as compared to the saline injection control.

FIGS. 11A and 11B show tumor response in an in vivo mouse model. FIG. 11A is a graph of the tumor size for the untreated control (line with circles) and for the NK cell composition and/or preparation (line with squares) over time for this study. The NK cell composition was able to cause a significant delay in tumor progression. FIG. 11B shows the NK cell composition persisted and was detectable 37 days post injection, as measured by detecting human CD45⁺ cells in the tumor.

FIGS. 12A-12D show expansion of HSPCs with Notch 1 antibody (see US Patent Application Publication No. 2017/0107493, incorporated herein by reference) was comparable to expansion using DXI.

FIGS. 13A and 13B show an NK cell composition and/or preparation (referred to as an NK Cell Product), K562, or A549 tumor cells were cultured alone, or together for three days. The study was run in triplicate. After three days, cytokine levels were determined by Luminex® assay. (The limit of detection of the assay used was 1 μg/mL of cytokine). Both IFNγ (FIG. 13A) and TNFα (FIG. 13B) levels increased when an NK cell composition and/or preparation was co-cultured in the presence of tumor cells. These results indicated the NK cells in the preparation became more active in the presence of tumor cells.

FIG. 14A-14E shows more consistent distribution of transgene expression across cell lineages was observed following transduction on Day 1, 4, 7, 14, or 21.

FIG. 15 shows the expression constructs included tEGFR as a selectable marker. S=Signal sequence from GM-CSF R; V_{L}=variable light chain; L=linker; V_{H}=variable heavy chain; H=Hinge spacer from human IgG₄; TM=CD28 or NKG2D transmembrane domain; 4-1BB=intracellular signaling domain; CD3zeta=intracellular signaling domain; 2A=self-cleaving peptide.

FIG. 16 shows the expression constructs included human IL-15 for support of NK cells. S=Signal sequence from GM-CSF R; V_{L}=variable light chain; L=linker; V_{H}=variable heavy chain; H=Hinge spacer from human IgG₄; TM=CD28 or NKG2D transmembrane domain; 4-1BB=intracellular signaling domain; CD3zeta=intracellular signaling domain; 2A=self-cleaving peptide.

FIGS. 17A and 17B show IL-15 added during the expansion phase to prime NK cell differentiation produces significant increases in CD56⁺ cells and that the process is reproducible. The Figures show the results from 4 batches of NK cell preparations produced at manufacturing scale. FIG. 17A shows the increase in CD56⁺ cells/starting CD34⁺ cells over the course of a 24-day culture (phases 1 and 2). FIG. 17B demonstrates the reproducibility of CD56⁺ cell generation over a 24-day cell culture process.

FIGS. 18A through 18C show the capacity of the NK cell preparations to serially kill repeat doses of target cells over a prolonged duration. FIG. 18A is a fresh NK cell preparation. FIG. 18B is a cryopreserved and thawed NK cell preparation, and FIG. 18C is an assay where a fresh NK cell preparation received repeat doses of target cells every 3 days for a total of 10 days.

FIG. 19 shows the activity of an NK cell preparation in a Kasumi-1 AML diffuse tumor model. The model was tested in NSG mice with or without NK cell preparation treatment by measuring Kasumi-1 tumor burden via bioluminescent imaging. Compared against buffer-injected control treatment, the NK cell preparation significantly inhibited tumor progression.

FIG. 20 shows NSG mouse survival in the Kasumi 1 AML model used in FIG. 19. The NK cell preparation significantly prolonged median survival in mice as compared with the mice injected with the buffer control.

FIG. 21 shows flow cytometry-based detection of tCD19 transgene expression as an indirect measure of CAR expression in transduced CD56⁺ and CD56⁻ cells. Plots show the frequency of CD56 expression and tCD19 expression in cells at day 28 of culture following transduction with a lentivirus vector expressing a mesothelin targeted CAR and a truncated CD19 (tCD19) extracellular domain driven by the EF1alpha promoter. Bulk cells were transduced using an MOI of 30, two times, 3 hours apart on day 7 of expansion.

FIG. 22 shows cytotoxic activity of MSLN CAR-NK cells or control NK cells against NOMO-1 or NOMO-1^{MSLN-/-} AML tumor cells in a 24-hour in vitro cytotoxicity assay. There is a significant and specific increase in tumor cell killing by the MSLN CAR-NK cells against the MSLN expressing NOMO-1 cells but not against the NOMO-1^{MSLN-/-} tumor cells. No difference in killing was observed with the control NK cells against the NOMO-1 cells+/−MSLN knockout.

FIG. 23 shows flow cytometry-based detection of tCD19 transgene expression as an indirect measure of CAR expression in enriched CD56⁺ cells. Plots show the frequency of CD56 expression and tCD19 expression (Tx⁺) in cells at day 27 of culture following transduction with a lentivirus vector expressing a mesothelin-targeted CAR and a truncated CD19 (tCD19) extracellular domain driven by the EF1alpha promoter. CD56⁺ cells were enriched by magnetic bead separation and transduced using an MOI of 40 on day 23 of culture in the differentiation phase.

FIG. 24 shows cytotoxic activity of CAR-NK cells or control NK cells against NOMO-1 cells or NOMO-1^{MSLN-/-} cells in a 24-hour in vitro cytotoxicity assay. There is a significant and specific increase in tumor cell killing by the MSLN CAR-NK cells against the MSLN expression NOMO-1 cells but not against the NOMO-1^{MSLN-/-} cells. No difference in killing was observed with the control NK cells against the NOMO-1 cells+/−the MSLN knockout

### DETAILED DESCRIPTION OF SPECIFIC EMBODIMENTS

Although any methods and materials similar or equivalent to those described herein can be used in the practice or testing of the present cell compositions and/or preparations, certain preferred methods and materials are described. For purposes of the present disclosure, the following terms are defined below.

As used herein, “expanded HSPCs” refers to hematopoietic stem cells or stem and progenitor cells that have been subjected to a technique for expanding the hematopoietic stem cells, or hematopoietic stem and progenitor cells ex vivo, which technique has been shown to result in (i) an increase in the number of hematopoietic stem cells, or hematopoietic stem and progenitor cells, in an aliquot of the cells thus expanded, or (ii) an increased number of severe combined immunodeficiency (SCID) repopulating cells determined by limiting-dilution analysis as shown by enhanced engraftment in non-obese diabetic (NOD)/SCID mice infused with an aliquot of the cells thus expanded. These are relative to that seen with an aliquot of the cells not subjected to the expansion technique. (See US Patent Application Publication No. 2013/0095079; Delaney et al., Nature Med. 16 (2): 232-236, 2010). Typically, the hematopoietic stem cells, or stem and progenitor cells, are CD34⁺. In some embodiments, the hematopoietic stem cells, or hematopoietic stem and progenitor cells, are derived from human umbilical cord blood and/or human placental blood. In some embodiments, the expanded stem cells are prepared using a Notch-agonist expansion method. In some embodiments, the expanded stem cells are prepared using a Delta1^{ext-IgG} (DXI) expansion method.

As used herein, a “chemotherapy regimen” refers to a regimen for chemotherapy, defining the drugs to be used, their dosage, the frequency and duration of treatments, and other considerations. Such regimens can combine several chemotherapy drugs in combination with chemotherapy. The majority of drugs used today in chemotherapy are cytostatic or cytotoxic.

The present disclosure provides compositions comprising a natural killer (NK) cell composition and/or preparation and methods of using such NK cell compositions and/or preparations for immunotherapy. The NK cell compositions and/or preparations can be used in therapies for a broad range of viral infections, malignancies, and other diseases, including hematologic and non-hematologic malignancies. An NK cell composition and/or preparation is produced from pooled, expanded CD34⁺ hematopoietic stem and progenitor cells (HSPCs). Typically, HSPCs from at least two or more, or up to at least four, different human donors are combined or pooled, before or after ex vivo expansion, to generate an expanded HSPC cell population. The expanded cell population is then differentiated ex vivo to generate an NK cell composition and/or preparation comprising a mixture of CD56⁺ and CD56⁻ cells. The CD56⁺ cells are predominantly NK cells. The CD56⁻ cells that make up a portion of the cell composition and/or preparation are typically of myeloid origin and support NK cell differentiation and activation during differentiation. The CD56⁻ cells produced during the ex vivo generation of the CD56⁺ are considered endogenous to the CD56⁺ cells.

In some embodiments, the NK cell composition and/or preparation comprises from about 50% up to about 80% CD56⁺ cells and about 20% to about 50% CD56⁻, endogenous myeloid-derived cells, such as dendritic cells, macrophages, and granulocytes. In some embodiments, the NK cell composition and/or preparation comprises from about 50% up to about 85% CD56⁺ cells and about 15% to about 50% CD56⁻, endogenous myeloid-derived cells, such as dendritic cells, macrophages, and granulocytes. In some embodiments, the NK cell composition and/or preparation comprises from about 55% up to about 65% CD56⁺ cells and about 35% to about 45% CD56⁻, endogenous myeloid-derived cells, such as dendritic cells, macrophages, and granulocytes. In some embodiments, the NK cell composition and/or preparation comprises from about 70% up to about 85% CD56⁺ cells and about 15% to about 30% CD56⁻, endogenous myeloid-derived cells, such as dendritic cells, macrophages, and granulocytes.

In addition to CD56, the CD56⁺ cells can express a high frequency of natural killer cell proteins NKp30, NKp46, NKp44, and NKG2A, and granzyme B; a moderate to high frequency of perforin and CD107a; a low to moderate frequency of NKG2D; and substantially no KIRs. In some embodiments the CD56⁺ cells are KIR⁻. (As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1). In some embodiments, CD16 is expressed at a low frequency. In some embodiments, CD16 is expressed at a moderate frequency. In some embodiments, the CD56⁻ cells express a moderate to high frequency of granzyme B, a high frequency of CD107a, and a low frequency of perforin.

In some embodiments, an NK cell composition and/or preparation produced by the methods described herein has the following characteristics.

The frequency of expression of the various NK markers is shown in the following Table.

TABLE 1
Frequency of NK Cell Marker Expression

| CD56⁺ Cells | | CD56⁻ Cells | |
|---|---|---|---|
| Granzyme B+ | High | Granzyme B⁺ | Moderate to high |
| Perforin⁺ | Moderate to high | Perforin⁺ | Low |
| CD107a⁺ | Moderate | CD107a⁺ | High |
| CD16⁺ | Low to moderate | CD15⁺CD14⁺ | Moderate |
| NKp30⁺ | High | CD15⁺CD14⁻ | Low to moderate |
| NKp44⁺ | High | CD15⁻CD14⁺ | Low to moderate |
| NKp46⁺ | High | CD11b⁺CD11V⁺ | High |
| NKG2A⁺ | High | of the CD15⁻CD14⁻ | |
| NKG2D⁺ | Low to moderate | | |
| KIR | Substantially no | | |

High expression refers to a frequency of about 60 to about 100%. Moderate expression refers to a frequency of about 20 to about 60%. Low expression refers to a frequency of about 1 to about 20%. Substantially no refers to a frequency of expression of less than 1%. As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1.

In some embodiments, the ranges of the markers for the CD56⁺ and CD56⁻ cells are as recited in Tables 2 and 3:

TABLE 2
Range of NK Cell Markers for CD56⁺ Cells

| CD56⁺ Cells | Frequency (Mean ± SD) | Range |
|---|---|---|
| CD56⁺ population | 60.7 ± 14.7 | 27.0-80.0 |

Subsets within CD56⁺ Cells

| | | |
|---|---|---|
| Granzyme B⁺ | 89.6 ± 7.1 | 79.1-98 |
| Perforin⁺ | 61.0 ± 12.3 | 51.9-81.5 |
| CD107a⁺ | 49.7 ± 5.7 | 42.9-58.8 |
| CD16⁺ | 18.4 ± 12.9 | 4.0-30.0 |
| NKp30⁺ | 84.1 ± 10.9 | 68.7-92.8 |
| NKp44⁺ | 80.0 ± 7.1 | 81.1-88.9 |
| NKp46⁺ | 85.4 ± 3.2 | 81.6-8.8 |
| NKG2A⁺ | 73.6 ± 2.0 | 70.4-85.4 |
| NKG2D⁺ | 7.3 ± 1.2 | 6.3-8.1 |
| KIR⁺ | 0.14 ± 0.04 | 0.10-0.16 |

TABLE 3
Range of NK Cell Markers for CD56⁻ Cells

| CD56⁻ Cells | Frequency (Mean ± SD) | Range |
|---|---|---|
| CD56⁻ population | 39.3 ± 14.7 | 20.0-73.0 |

Subsets within CD56⁻ population

| | | |
|---|---|---|
| Granzyme B⁺ | 64.4 ± 21.6 | 27.4-82.3 |
| Perforin⁺ | 1.5 ± 0.3 | 1.2-2.0 |
| CD107a⁺ | 89.3 ± 4.6 | 82.7-95.0 |
| CD15⁺CD14⁺ | 28.7 ± 3.7 | 24.3-33.7 |
| CD15⁺CD14⁻ | 19.8 ± 5.4 | 15.3-28.9 |
| CD15⁻CD14⁺ | 18.3 ± 9.0 | 21.4-2.9 |
| CD11b⁺CD11c⁺ (within the CD15⁻CD14⁻ population) | 76.6 ± 6.4 | 70.3-81.4 |

In some embodiments, an NK cell composition and/or preparation contains less than 2% CD3⁺ cells (T cells). In some embodiments, the NK cell composition and/or preparation contains less than 1% CD3⁺ cells (T cells). In some embodiments, the NK cell composition and/or preparation contains less than 2% CD19⁺ cells (B cells). In some embodiments, the NK cell composition and/or preparation contains less than 1% CD19⁺ cells (B cells). In some embodiments, the NK cell composition and/or preparation contains less than 2% CD34⁺ cells. In some embodiments, the NK cell composition and/or preparation contains less than 1% CD34⁺ cells (HSPCs). In some embodiments, the NK cell composition and/or preparation contains less than 2% CD19⁺ cells (B cells), less than 2% CD3⁺ (T cells) and less than 2% CD34⁺ cells (HSPCs). In some embodiments, the NK cell composition and/or preparation contains less than 1% CD19⁺ cells, less than 1% CD3⁺ (T cells) and less than 1% CD34⁺ cells (HSPCs).

In humans, NK cells are regulated by clonally distributed killer immunoglobulin-like receptors (KIRs) that recognize allotypic determinants displayed by different human leukocyte antigen (HLA) class I alleles. Inhibitory KIRs are generally dominant and prevent NK cells from killing autologous cells. The NK cell compositions and/or preparations prepared by the present methods lack MHC class I inhibitory signals. While expressing high levels of NKG2A, the NK cell composition and/or preparation can be more active in vitro than adult NK cells.

The receptor phenotype of the NK cell compositions and/or preparations produced by the methods disclosed herein can further include a high frequency of expression of the natural cytotoxicity receptors NKp30, NKp44, NKp46, while having a low to moderate frequency of expression of NKG2D by the CD56⁺ cells. The CD56⁺ cells of the instant NK cell compositions and/or preparations can further be identified by low to moderate CD16 expression.

The differentiated NK cells of the present disclosure are further identified by their capability to lyse various tumor cells lines at levels similar to or greater than activated peripheral blood NK cells (as further described in the Examples). The cytotoxic capabilities of the cells in culture can therefore be measured to verify the characteristics of an NK cell composition and/or preparation.

Preparation of differentiated NK cells ex vivo as described in the methods of the present disclosure comprises two separate phases, wherein there is a first phase involving expansion of HSPCs to generate expanded HSPCs, and wherein the first phase is followed by a second phase during which the expanded HSPCs are differentiated to form an NK cell composition and/or preparation. In contrast to the prior art, feeder cells are not used in either phase.

The expanded HSPCs comprise hematopoietic stem cells or stem and progenitor cells that have been expanded ex vivo (e.g., with a Notch ligand) and depleted of both T cells and red blood cells. The expanded HSPCs are typically CD34⁺ hematopoietic stem cells or stem and progenitor cells and are typically derived from different human sources which are not HLA-matched. In some embodiments, the expanded HSPCs are CD34⁺ hematopoietic stem or stem and progenitor cells from different human umbilical cord blood sources and/or placental blood sources. The hematopoietic stem or stem and progenitor cells comprise multiple HLA-types because the HSPCs are not matched to each other prior to pooling. As used herein, depleted of T cells refers to less than 2% CD3⁺ cells (T cells), or less than 1% CD3⁺ cells (T cells), or less than 0.5% CD3⁺ cells (T cells), or less than 0.1% CD3⁺ cells (T cells), in the expanded HSPCs.

In certain embodiments the hematopoietic stem cells or hematopoietic stem and progenitor cells have been immunologically matched to the patient that is to receive the NK cell composition and/or preparation as an immunotherapy. The immunological matching can be a full match or a partial mismatch of up to 2, 3, or sometimes 4 immunotypes. In the majority of compositions comprising the hematopoietic stem cells or hematopoietic stem and progenitor cells have been matched at the most common HLA antigens, such as, for example, HLA A-2, HLA-B7, and the like, or high frequency combinations by HLA linkage. The choice of matching will be selected depending on the patient population to be treated.

In some embodiments, the CD34⁺ hematopoietic stem cells or hematopoietic stem and progenitor cells are derived from cord blood and/or from placental blood (human cord blood or human placental blood). Such blood can be obtained by methods known in the art. See, e.g., U.S. Pat. Nos. 5,004,681 and 7,147,626 and US Patent Application Publication No. 2013/0095079 for a discussion of collecting cord and placental blood at the birth of a human. Umbilical cord blood and/or human placental blood collections are made under sterile conditions. Upon collection, cord and/or placental blood can be mixed with an anticoagulant, such as CPD (citrate-phosphate-dextrose), ACD (acid citrate-dextrose), Alsever's solution (Alsever et al., N. Y. St. J. Med. 41:126, 1941), De Gowin's Solution (De Gowin, et al., J. Am. Med. Ass. 114:850, 1940), Edglugate-Mg (Smith, et al., J. Thorac. Cardiovasc. Surg. 38:573, 1959), Rous-Turner Solution (Rous and Turner, J. Exp. Med. 23:219, 1916), other glucose mixtures, heparin, ethyl biscoumacetate, and the like. See, generally, Hurn, Storage of Blood, Academic Press, New York, pp. 26-160, 1968). In one embodiment, ACD can be used.

Cord blood can preferably be obtained by direct drainage from the umbilical cord and/or by needle aspiration from the delivered placenta at the root and at distended veins. Preferably, the collected human cord blood and/or placental blood is free of contamination (e.g., bacteria or viral) and, in particular, viral contamination.

Prior to collection of the cord blood, a maternal health history can be determined to identify risks that the cord blood cells might pose, e.g., transmitting genetic or infectious diseases, such as cancer, a leukemia, immune disorders, neurological disorders, hepatitis or HIV/AIDS. The collected cord blood can have undergone testing, for example, for one or more of cell viability, HLA typing, ABO/Rh typing, CD34⁺ cell count, and total nucleated cell count.

Once the umbilical cord blood and/or placental blood is collected from human donors at birth, the blood is processed to produce enriched HSPCs. Preferably, the HSPCs are CD34⁺ cells or predominantly CD34⁺ cells. The HSPCs are typically depleted of T cells and of red blood cells, resulting in enriched HSPCs. As used herein, depletion of T cells refers to the presence of less than about 2% CD3⁺ cells, less than about 1% CD3⁺ cells, or less than about 0.5% CD3⁺ cells, or less than about 0.1% CD3⁺ cells. Enrichment thus refers to a process wherein the percentage of HSPCs in the cell population is increased (relative to the percentage in the population before the enrichment procedure). Purification is one example of enrichment.

Prior to processing for enrichment, the collected cord and/or placental blood can be fresh or may have been previously cryopreserved. Any suitable technique known in the art for cell separation/selection can be used to carry out the enrichment for HSPCs. Methods which rely on differential expression of cell surface markers can be used. For example, cells expressing the cell surface marker CD34 can be positively selected using a monoclonal antibody specific to CD34, such that cells expressing CD34 are separated from cells not expressing CD34. Moreover, the separation techniques employed preferably maximize the viability of the cells to be selected. The particular technique employed depends upon the efficiency of separation, the cytotoxicity of the methodology, ease and speed of performance, and necessity for sophisticated equipment and/or technical skill.

Procedures for separation can include magnetic separation, using antibody-coated magnetic beads, affinity chromatography, and “panning” with antibody attached to a solid matrix, e.g., a plate, or another convenient technique. Techniques providing accurate separation/selection include fluorescence activated cell sorters, which can have varying degrees of sophistication, e.g., a plurality of color channels, low angle and obtuse light scattering detecting channels, impedance channels, and the like.

The antibodies used in the selection process allow for ease of separation of the particular cell type, and can be conjugated with markers, such as magnetic beads, which allow for direct separation; biotin, which enables removal by adhering to avidin or streptavidin bound to a support; fluorochromes, which can be used with a fluorescence activated cell sorter; or the like. Any technique can be employed which is not unduly detrimental to the viability of the remaining cells.

In a preferred embodiment, fresh cord blood units or frozen and thawed cord blood units are processed to enrich for CD34⁺ HSPCs using anti-CD34 antibodies directly or indirectly conjugated to magnetic particles in connection with a magnetic cell separator, for example, the CliniMACS® Cell Separation System (Miltenyi Biotec, Bergisch Gladbach, Germany), which employs nano-sized super-paramagnetic particles composed of iron oxide and dextran coupled to specific monoclonal antibodies. The CliniMACS® Cell Separator is a closed sterile system, outfitted with a single-use disposable tubing set. The disposable tubing set can be used for, and discarded after, processing a single unit of collected cord and/or placental blood to enrich for CD34⁺ HSPCs.

In a typical embodiment, two or more, or up to at least four or more umbilical cord blood and/or placental blood units, can be pooled prior to enriching for HSPCs. In another embodiment, individual populations of CD34⁺ HSPCs can be pooled after enriching for the HSPCs. In specific embodiments, the number of umbilical cord blood and/or placental blood units, or populations of HSPCs, that are pooled is 2, 4, 5, 6, 7, 8, 9, 10, 15, 20, 25, 30, 35, or 40, or at least any of the foregoing numbers. The number of individual populations of HSPCs pooled can depend on, for example, the number of cells in each individual populations, and/or the number of cells required for a particular patient and the immunotherapy intended. In some embodiments, the pool contains 2 to 8, 4 to 8, 2 to 10, 4 to 10, 4 to 20, or 4 to 25, and no more than 20 or 25, umbilical cord blood and/or placental blood units, or CD34⁺ HSPC populations. In a typical embodiment the umbilical cord blood and/or placental blood units or hematopoietic stem or stem and progenitor cell populations can be pooled without regard to the HLA-type of the HSPCs. In some embodiments, the cells in the pool are combined without regard to race or ethnicity. In some embodiments, the cells in the pool are derived from the umbilical cord blood and/or placental blood of individuals of the same race, e.g., African-American, Caucasian, Asian, Hispanic, Native-American, Australian Aboriginal, Inuit, Pacific Islander, or derived from umbilical cord blood and/or placental blood of individuals of the same ethnicity, e.g., Irish, Italian, Indian, Japanese, Chinese, Russian, and the like. In less common embodiments, the cells can be from an individual cell population that has been matched or partially matched to the patient. If there are a sufficient number of matched or partially matched units available the units can be pooled; however, given the sometimes-difficult process to find units that match a particular patient, pooling is commonly not carried out for matched or mismatched units.

Typically, prior to enrichment for HSPCs, the red blood cells and white blood cells of the cord blood and/or placental blood are separated. In some embodiments, depletion of red blood cells refers to the separation of red blood cells from white blood cells. Once the separation of the red blood cells and the white blood cells has taken place, the red blood cell fraction can be discarded, and the white blood cell fraction can be processed, for example, in the magnetic cell separator as described above to enrich for CD34⁺ HSPCs. Separation of the white and red blood cell fractions can be performed by any method known in the art, including, for example, centrifugation techniques. Other separation methods that can be used include the use of commercially available products FICOLL™ or FICOLL-PAQUE™ or PERCOLL™ (GE Healthcare, Piscataway, New Jersey). FICOLL-PAQUE™ is normally placed at the bottom of a conical tube, and the whole blood is layered above. After being centrifuged, the following layers will be visible in the conical tube, from top to bottom: plasma and other constituents, a layer of mononuclear cells, called a buffy coat, containing the mononuclear cells (white blood cells), and erythrocytes and granulocytes, which should be present in pellet form. This separation technique allows easy harvest of the mononuclear cells.

Optionally, prior to CD34⁺ cell selection, an aliquot of the cord blood and/or placental unit can be checked for a total nucleated cell count and/or CD34⁺ cell content. In a specific embodiment, after the CD34⁺ cell selection, both CD34⁺ and CD34⁻ cell fractions are recovered. Optionally, DNA can be extracted from a sample of the CD34⁻ cell fraction for initial HLA typing and future chimerism studies, even though HLA matching of the CD34⁺ cells to the other cord blood and/or placental blood cells is not typically performed.

The CD34⁺ enriched HSPCs can be subsequently processed prior to expansion, for example, by suspension in an appropriate cell culture medium for storage or transport. In a preferred embodiment, the cell culture medium is a cell culture medium suitable for the maintenance of viability of CD34⁺ HSPCs. For example, the cell culture medium can be a serum-free, cytokine-free, hematopoietic stem cell or stem and progenitor cell culture medium to which growth factors are added, for example, at the following concentrations: 50-300 ng/ml of stem cell factor (SCF), 50-300 ng/ml of Flt-3 receptor ligand (Flt3L), 50-100 ng/ml of thrombopoietin (TPO), 50-100 ng/ml of interleukin-6 (IL-6), and 10 ng/ml of interleukin-3 (IL-3). In more specific embodiments, the cell culture medium contains 300 ng/ml of stem cell factor, 300 ng/ml of Flt-3 receptor ligand, 100 ng/ml of TPO, 100 ng/ml of IL-6 and 10 ng/ml of IL-3; or 50 ng/ml of SCF, 50 ng/ml of Flt-3L, 50 ng/mL of TPO, 50 ng/ml of IL-6 and 10 ng/mL of IL-3. In another preferred embodiment, the cell culture medium includes, or alternatively consists of, a serum free, hematopoietic stem cell or stem and progenitor cell culture medium (e.g., STEMSPAN™ Serum Free Expansion Medium or STEMSPAN™ Serum Free Expansion Medium II (StemCell Technologies, Vancouver, British Columbia)) supplemented with 10 ng/ml recombinant human Interleukin-3 (rhIL-3), 50 ng/ml recombinant human Interleukin-6 (rhIL-6), 50 ng/mL recombinant human Thrombopoietin (rhTPO), 50 ng/mL recombinant human Flt-3 Ligand (rhFlt-3L), 50 ng/ml and recombinant human stem cell factor (rhSCF). In another preferred embodiment, the cell culture medium consists of a serum-free hematopoietic stem cell or stem and progenitor cell culture medium (e.g., StemSpan Serum Free Expansion Medium II (SFEM II, StemCell Technologies, Vancouver, British Columbia)) supplemented with recombinant human rhSCF, rhFlt-3L, rhTPO, rhIL-6 (each at 50 ng/mL final concentration), and rhIL-3 (at 10 ng/ml final concentration).

In a specific embodiment, the umbilical cord blood and/or placental blood units are red blood cell depleted, and the number of CD34⁺ cells in the red blood cell depleted fraction is determined. In some embodiments, depletion of red blood cells refers to separation of red blood cells from white blood cells or separation of red blood cells from CD34⁺ cells. Preferably, umbilical cord blood and/or placental blood units containing more than 3.5 million CD34⁺ cells are subject to the enrichment methods described above.

After the HSPCs have been isolated (e.g., from human cord blood and/or human placental blood collected from humans at birth) according to the enrichment methods described above or other methods known in the art, the enriched HSPCs are expanded to increase the number of HSPCs, e.g., CD34⁺ HSPCs. The HSPCs are cultured in an expansion culture medium under cell growth conditions (e.g., promoting mitosis) such that the HSPCs grow and divide (proliferate) to obtain an expanded CD34⁺ HSPC population. During expansion of the HSPCs, minimal differentiation of HSPCs to NK cells occurs (i.e., less than 2% or less than 1% of the resulting cells are NK cells). In one embodiment, individual populations of HSPCs each derived from umbilical cord blood and/or placental blood of a single human at birth can be pooled, without matching to the HLA type of the other HSPCs, prior to or after expansion. In another embodiment, the HSPCs are expanded prior to pooling. Preferably, the technique used for expansion is one that has been shown to result in an increase in the number of hematopoietic stem cells, or hematopoietic stem and progenitor cells, e.g., CD34⁺ cells, in the expanded HSPCs relative to the unexpanded population of HSPCs, where the unexpanded cell population and expanded cell population are from different aliquots of the same source of HSPCs, wherein the expanded HSPCs but not the unexpanded HSPCs are subjected to the expansion technique.

Expansion techniques include, but are not limited to, those described in U.S. Pat. No. 7,399,633 B2; US Patent. Application Publication No. 2013/0095079; Delaney et al., Nature Med. 16 (2): 232-236, 2010 (incorporated herein by reference); as well as those described below.

In some embodiments, the HSPCs are cultured in vitro or ex vivo in an expansion culture medium, which is serum free and suitable for culture of hematopoietic stem cell or stem and progenitor cells, in the presence of growth factors, and are exposed to cell growth conditions (e.g., promoting mitosis) such that the HSPCs proliferate to generate an expanded population of HSPCs.

In an exemplary embodiment, the expansion culture medium, suitable for expansion of hematopoietic stem cell or stem and progenitor cells, is a serum free, culture medium such as Iscove's MDM containing non-animal sourced BSA, recombinant human insulin, human transferrin, 2-mercaptoethanol, and other supplements, with a Notch ligand and the growth factors, as described below. In other embodiments, the hematopoietic stem cell or stem and progenitor cell culture medium is STEMSPAN™ Serum Free Expansion Medium (StemCell Technologies, Vancouver, British Columbia), or STEMSPAN™ Serum Free Expansion Medium II (StemCell Technologies, Vancouver, British Columbia).

In some embodiments, the HSPCs are cultured in the presence of an amount of a Notch ligand (i.e., an agonist of Notch function effective to inhibit differentiation), typically an immobilized agonist of Notch function, in an expansion culture medium and are exposed to cell growth conditions (e.g., promoting mitosis) such that the HSPCs proliferate to generate an expanded HSPC population. Differentiation of HSPCs to NK cells is minimized during the expansion phase (i.e., less than 2% or less than 1% of the resulting cells are NK cells). In a more preferred embodiment, the expansion culture medium contains an amount of an agonist of Notch function effective to inhibit differentiation and growth factors, and the HSPCs are exposed to cell growth conditions (e.g., promoting mitosis) such that the hematopoietic stem or stem and progenitor cells proliferate to obtain an expanded hematopoietic stem or stem and progenitor cell population. The expanded hematopoietic stem or stem and progenitor cell population is typically transferred to the differentiation cell culture medium following expansion. Optionally, the Notch ligand is inactivated or removed from the expanded HSPC cell population prior to the differentiation phase (e.g., by separation or dilution).

In some embodiments, the hematopoietic stem or stem and progenitor cells are cultured for expansion for 2, 3, 4, 5, 6, 7, 8, 9, 10, 11, 12, 13, 14, 15, 16, 17, 18, 19, 20, 21, 22, 23, 24, or 25 days or more; or, preferably, the hematopoietic stem or stem and progenitor cells are cultured for expansion for at least 10 days or from about 7 to about 14 days. In some embodiments, the hematopoietic stem or stem and progenitor cells are cultured for about 7 days, about 14 days, about 13 days or about 15 days. It should be noted that the duration of the expansion would be determined by the ability to achieve either the desired or maximal cell numbers while retaining stem and progenitor cell immunophenotypic qualities/characteristics needed for the generation of the desired cell preparation. Expansion would be stopped if cell proliferation stopped, viability declined, or if the cell began to substantially lose the qualities/characteristics of HSPCs.

An exemplary ex vivo culture condition for expanding the hematopoietic stem or stem and progenitor cells includes culturing the cells for 7 to 14 days in the presence of fibronectin fragments and the extracellular domain of a Delta protein fused to the Fc domain of human IgG (Delta1^{ext-IgG}) in a serum free, expansion culture medium supplemented with the following human growth factors: SCF, Flt-3L, TPO, IL-6 and IL-3. Preferably, the foregoing growth factors are present at the following concentrations: 50-300 ng/mL SCF, 50-300 ng/ml Flt-3L, 50-100 ng/mL TPO, 50-100 ng/ml IL-6 and 10 ng/ml IL-3. In more specific embodiments, 300 ng/mL SCF, 300 ng/ml of Flt-3L, 100 ng/mL TPO, 100 ng/ml IL-6 and 10 ng/ml IL-3, or 50 ng/mL SCF, 50 ng/ml of Flt-3L, 50 ng/ml TPO, 50 ng/ml IL-6 and 10 ng/ml IL-3, are used. In a more preferred embodiment, the expansion culture medium (e.g., STEMSPAN™ Serum Free Expansion Medium (StemCell Technologies, Vancouver, British Columbia)) contains, or consists of, 10 ng/mL recombinant human Interleukin-3 (rhIL-3), 50 ng/mL rhIL-6, 50 ng/mL rhTPO, 50 ng/mL rhFlt-3L, 50 ng/mL and rhSCF. In another more preferred embodiment, the expansion culture medium (e.g., StemSpan™ Serum Free Expansion Medium II (SFEM II, StemCell Technologies, Vancouver, British Columbia)) contains, or consists of, rhSCF, rhFlt-3L, rhTPO, rhIL-6 (each at 50 ng/mL final concentration), and rhIL-3 (at 10 ng/ml final concentration).

In some embodiments, the Notch ligand is DXI (Delta1^{ext-IgG}) and the expansion phase is performed as follows: Delta1^{ext-IgG} (DXI) is immobilized on the surface of the cell culture dish(es). In a specific embodiment, the cell culture dishes are coated overnight at 4° C. (or for a minimum of 2 hours at 37° C.) with 2.5 μg/mL Delta1^{ext-IgG} and 5 μg/mL RetroNectin® (a recombinant human fibronectin fragment also referred to as rFN-CH-296) in phosphate buffered saline, before adding the enriched hematopoietic stem or stem and progenitor cells. Preferably the expansion culture medium (e.g., STEMSPAN™ Serum Free Expansion Medium or StemSpan™ Serum Free Expansion Medium II (StemCell Technologies, Vancouver, British Columbia)) is supplemented with 10 ng/mL rhIL-3, 50 ng/mL rhIL-6, 50 ng/mL rhTPO, 50 ng/mL rhFlt-3L, and 50 ng/mL rhSCF.

In some embodiments, the expansion culture medium does not include growth factors other than rhIL-3, rhIL-6, rhTPO, rhFlt-3L, and rhSCF. In some embodiments, the expansion culture medium does not contain the following added growth factors or cytokines: IL-7, GM-CSF, G-CSF, LIF, MIP-1α, IL-2 or IL-15. In some embodiments, the expansion culture medium does not contain the following added growth factors or cytokines: IL-7, GM-CSF, G-CSF, LIF, MIP-1a, or IL-2. In some embodiments, the expansion culture medium does not contain an aryl hydrocarbon receptor antagonist, such as those described in U.S. Pat. No. 9,175,266 or US Patent Application Publication No. 2018/0237749; both incorporated herein by reference.

After expansion of the hematopoietic stem or stem and progenitor cells, the total number of cells and viable CD34⁺ cells can be determined. For example, at Day 14 during expansion, a sample can be taken for determination of the total viable nucleated cell count. In addition, the total number of CD34⁺ cells can be determined by multi-parameter flow cytometry, and thus the percentage of CD34⁺ cells in the sample determined. Typically, cultures that have not resulted in at least a 10-fold increase in the absolute number of CD34⁺ cells are discontinued. In a preferred embodiment, those populations containing less than 50 million CD34⁺ viable cells can be discarded.

Viability can be determined by any method known in the art, for example, by trypan blue exclusion or 7-AAD exclusion. The percentage of viable CD34⁺ cells can be assessed by flow cytometry and use of a stain that is excluded by viable cells. The percentage of viable CD34⁺ cells=the number of CD34⁺ cells that exclude 7-AAD (or other appropriate stain) in an aliquot of the sample divided by the total cell number (TNC; both viable and non-viable) of the aliquot. Viable CD34⁺ cells in the sample can be calculated as follows: Viable CD34⁺ cells=TNC of sample x % viable CD34⁺ cells in the sample. The proportional increase during enrichment or expansion in viable CD34⁺ cells can be calculated as follows: Total Viable CD34⁺ cells Post-culture/Total Viable CD34⁺ cells Pre-culture.

In some embodiments, the hematopoietic stem or stem and progenitor cells are expanded by culturing the cells ex vivo in an expansion culture medium and in the presence of an agonist of Notch function and one or more growth factors or cytokines for a given period of time, as described above. An agonist of Notch function, also referred to as Notch agonist or Notch ligand, is an agent that promotes, i.e., causes or increases, activation of Notch pathway function. As used herein, “Notch function” means a function mediated by the Notch signaling (signal transduction) pathway, including but not limited to nuclear translocation of the intracellular domain of Notch, nuclear translocation of RBP-Jκ or its Drosophila homolog Suppressor of Hairless; activation of bHILH genes of the Enhancer of Split complex, e.g., Mastermind; activation of the HES-1 gene or the KBF2 (also called CBF1) gene; inhibition of Drosophila neuroblast segregation; and binding of Notch to Delta, Jagged/Serrate, Fringe, Deltex or RBP-Jκ/Suppressor of Hairless, or homologs or analogs thereof. See generally the review article by Kopan et al., Cell 137:216-233, 2009 for a discussion of the Notch signal transduction pathway and its effects upon activation; see also Jarriault et al., Mol. Cell. Biol. 18:7423-7431, 1998.

Notch activation is carried out by exposing a cell to a Notch agonist. The agonist of Notch function can be but is not limited to a molecule immobilized on a solid phase. Exemplary Notch agonists are the extracellular binding ligands Delta and Serrate which bind to the extracellular domain of Notch and activate Notch signal transduction, or a fragment of Delta or Serrate that binds to the extracellular domain of Notch and activates Notch signal transduction. Nucleic acid and amino acid sequences of Delta and Serrate have been isolated from several species, including human are known in the art, and are disclosed in International Patent Publication Nos. WO 93/12141, WO 96/27610, WO 97/01571, and Gray et al., Am. J. Path. 154:785-794, 1999. (All incorporated herein by reference in their entirety).

In a preferred embodiment, the Notch agonist is an immobilized fragment of a Delta or Serrate protein consisting of the extracellular domain of the protein fused to a myc epitope tag (Delta^{ext-myc} or Serrate^{ext-myc}, respectively), or an immobilized fragment of a Delta or Serrate protein consisting of the extracellular domain of the protein fused to the Fc portion of IgG (Delta^{ext-IgG} or Serrate^{ext-IgG} respectively). Notch agonists include but are not limited to Notch proteins and analogs and derivatives (including fragments) thereof; proteins that are other elements of the Notch pathway and analogs and derivatives (including fragments) thereof; antibodies thereto and fragments or other derivatives of such antibodies containing the binding region thereof; nucleic acids encoding the proteins and derivatives or analogs; as well as proteins and derivatives and analogs thereof which bind to or otherwise interact with Notch proteins or other proteins in the Notch pathway such that Notch pathway activity is promoted. Such agonists include but are not limited to Notch proteins and derivatives thereof comprising the intracellular domain, Notch nucleic acids encoding the foregoing, and proteins comprising the Notch-interacting domain of Notch ligands (e.g., the extracellular domain of Delta or Serrate). Other agonists include but are not limited to RBPJκ/Suppressor of Hairless or Deltex. Fringe can be used to enhance Notch activity, for example in conjunction with Delta protein. These proteins, fragments and derivatives thereof can be recombinantly expressed and isolated or can be chemically synthesized.

In yet another embodiment, the agonist of Notch is a peptidomimetic or peptide analog or organic molecule that binds to a member of the Notch signaling pathway. Such an agonist can be identified by binding assays selected from those known in the art, for example the cell aggregation assays described in Rebay et al., Cell 67:687-699, 1991 and in International Patent Publication No. WO 92/19734. (Both incorporated herein by reference).

In a preferred embodiment the agonist is a protein consisting of at least a fragment of a protein encoded by a Notch-interacting gene which mediates binding to a Notch protein or a fragment of Notch, which fragment of Notch contains the region of Notch responsible for binding to the agonist protein, e.g., epidermal growth factor-like repeats 11 and 12 of Notch. Notch interacting genes, as used herein, shall mean the genes Notch, Delta, Serrate, RBPJκ, Suppressor of Hairless and Deltex, as well as other members of the Delta/Serrate family or Deltex family which may be identified by virtue of sequence homology or genetic interaction and more generally, members of the “Notch cascade” or the “Notch group” of genes, which are identified by molecular interactions (e.g., binding in vitro, or genetic interactions (as depicted phenotypically, e.g., in Drosophila). Exemplary fragments of Notch-binding proteins containing the region responsible for binding to Notch are described in U.S. Pat. Nos. 5,648,464; 5,849,869; and 5,856,441. (All incorporated herein by reference).

The Notch agonists utilized by the methods described herein can be obtained commercially, produced by recombinant expression, or chemically synthesized.

In a specific embodiment, exposure of the cells to a Notch agonist is not done by incubation with other cells recombinantly expressing a Notch ligand on the cell surface (e.g., a feeder layer), but rather is by exposure to a cell-free Notch ligand, e.g., incubation with a cell-free ligand of Notch, which ligand is immobilized on the surface of a solid phase, e.g., immobilized on the surface of a tissue culture dish.

In specific embodiments, Notch activity is promoted by the binding of a Notch ligand(s) (e.g., Delta, Serrate) to the extracellular portion of the Notch receptor. Notch signaling appears to be triggered by the physical interaction between the extracellular domains of Notch and its ligands that are either membrane-bound on adjacent cells or immobilized on a solid surface. Full length ligands are agonists of Notch, as their expression on one cell triggers the activation of the pathway in the neighboring cell which expresses the Notch receptor. Soluble truncated Delta or Serrate molecules, comprising the extracellular domains of the proteins or Notch-binding portions thereof, that have been immobilized on a solid surface, such as a tissue culture plate, are particularly preferred Notch pathway agonists. Such soluble proteins can be immobilized on a solid surface by an antibody or interacting protein, for example an antibody directed to an epitope tag with which Delta or Serrate is expressed as a fusion protein (e.g., a myc epitope tag, which is recognized by the antibody 9E10) or a protein which interacts with an epitope tag with which Delta or Serrate is expressed as a fusion protein (e.g., an immunoglobulin epitope tag, which is bound by Protein A).

In another specific embodiment, and as described in U.S. Pat. No. 5,780,300 to Artavanis-Tsakonas et al., Notch agonists include reagents that promote or activate cellular processes that mediate the maturation or processing steps required for the activation of Notch or a member of the Notch signaling pathway, such as the furin-like convertase required for Notch processing, Kuzbanian, the metalloprotease-disintegrin (ADAM) thought to be required for the activation of the Notch pathway upstream or parallel to Notch (Schlondorff and Blobel, J. Cell Sci. 112:3603-3617, 1999), or, more generally, cellular trafficking and processing proteins such as the rab family of GTPases required for movement between cellular compartments (for a review on Rab GTPases, see Olkkonen and Stenmark, Int. Rev. Cytol. 176:1-185, 1997). The agonist can be any molecule that increases the activity of one of the above processes, such as a nucleic acid encoding a furin, Kuzbanian or rab protein, or a fragment or derivative or dominant active mutant thereof, or a peptidomimetic or peptide analog or organic molecule that binds to and activates the function of the above proteins.

U.S. Pat. No. 5,780,300 further discloses classes of Notch agonist molecules (and methods of their identification) which can be used to activate the Notch pathway, for example molecules that trigger the dissociation of the Notch ankyrin repeats with RBP-Jκ, thereby promoting the translocation of RBP-Jκ from the cytoplasm to the nucleus.

In some preferred embodiments, an expansion method including DXI is used. The Notch agonist DXI is an immobilized fragment of a Delta consisting of the extracellular domain of the protein fused to the Fc portion of IgG (Delta^{ext-IgG} or DXI), as described in U.S. Pat. No. 7,399,633 or an immobilized Notch-1 or Notch-2 specific antibody, as described in U.S. Pat. No. 10,208,286. Preferably, Delta1^{ext-IgG} is immobilized on the surface of the cell culture dishes. In a specific embodiment, the cell culture dishes are coated overnight at 4° C. (or for a minimum of 2 hours at 37° C.) with 2.5 μg/mL Delta1^{ext-IgG} and 5 μg/mL RetroNectin® (a recombinant human fibronectin fragment also referred to as rFN-CH-296) in phosphate buffered saline, before adding the hematopoietic stem or stem and progenitor cells. Preferably, the cell culture medium is a serum free hematopoietic stem cell culture medium (e.g., STEMSPAN™ Serum Free Expansion Medium or STEMSPAN™ Serum Free Expansion Medium II (StemCell Technologies, Vancouver, British Columbia)) supplemented with 10 ng/mL rhIL-3, 50 ng/mL rhIL-6, 50 ng/mL rhTPO, 50 ng/mL rhFlt-3L, and 50 ng/mL rhSCF. The hematopoietic stem or stem and progenitor cells are cultured in this embodiment for 7 to 14 days.

In certain embodiments, the increase in the number of CD34⁺ cells as a percentage of cells in the expanded HSPCs, relative to the cell population prior to the enrichment procedure, is at least 25-, 50-, 75-, 100-, 150-, 200-, 250-, 300-, 350-, 400- or at least 350-fold, and preferably is 100-200 fold or 100-400 fold.

Once the expanded hematopoietic stem cells or stem and progenitor cells are obtained, the expanded stem or stem and progenitor cell population (expanded HSPCs) can be collected and cryopreserved or can be used directly in the second phase to produce the NK cell composition and/or preparation.

The NK cell composition and/or preparation results from differentiation of the expanded HSPCs in a second phase of the process. In the differentiation phase, the HSPCs are cultured in a differentiation culture medium that contains an amount of one or more cytokine(s) effective to induce and direct HSPC differentiation into NK cells.

In some embodiments, the cytokines in the differentiation culture medium are IL-2 and IL-15. In some embodiments, the only cytokines in the differentiation culture medium are IL-2 and IL-15. In some embodiments, the amount of IL-2 and IL-15 in the differentiation culture medium is from about 25 U/mL to about 100 U/mL for IL-2 and from about 25 ng/mL to about 50 ng/ml for IL-15. In some embodiments, the amount of IL-2 and IL-15 in the differentiation culture medium is about 50 U/mL for IL-2 and about 40 ng/mL for IL-15. In some embodiments, the cytokines in the differentiation culture comprise IL-2 and IL-15, wherein other cytokines, such as Flt-3L, FGF-2, IL-6, IL-7, IL-12, IL-3, GM-CSF, G-CSF, LIF, MIP-1a, SCF, IL-21, IL-18, and 4-1BBL (4-1BB ligand), are not added to the differentiation culture medium. In some embodiments, the differentiation culture medium does not contain added cytokines other than IL-2 and IL-15.

In an embodiment, the differentiation culture medium further includes a supplement, such as, for example, human serum or plasma, or another proteinaceous fluid that provides a broad spectrum of macromolecules, carrier proteins for lipoid substances and trace elements, attachment and spreading factors, hormones, and growth factors that promote cell growth and health. In very early cell culture methods for hematopoietic stem cells fetal bovine serum (FBS) was used as this cell culture supplement; however, bovine proteins are not acceptable in human pharmaceutical products. As such, various human biological fluids have been used including, for example, human serum, plasma, fresh frozen plasma, platelet lysate, and the like. In certain embodiments of the present methods human AB serum has been used. In a particularly preferred embodiment platelet lysate has been used. In some embodiments, between about 2.5% to about 10% human platelet lysate is included in the differentiation medium. For example, the differentiation medium can comprise about 3%, about 4%, about 5%, about 6%, about 7%, about 8%, about 9%, or more than about 9% of human platelet lysate. In embodiments comprising human platelet lysate, the human platelet lysate is produced from human platelets (e.g. from platelet-rich plasma (PRP), expired platelets collected via apheresis, and the like) by processes such as, but not limited to, freezing and thawing cycles (e.g. between 1 and 6 cycles), sonication, solvent/detergent treatment, or activation with calcium or thrombin. Human platelets can be derived from autologous collections and/or allogeneic collections from multiple different individuals. The human platelet lysates can be used with or without regard to factors which affect the composition and bioactivity, such as, but not limited to, plasma content, growth factor content, donor age and gender, platelet counts, production process, heparin or anticoagulant presence, fibrinogen-depletion, cellular component presence, metabolite presence, blood group, storage conditions (e.g. duration and/or temperature), treatment to reduce/inactivate pathogens/viruses, and the like. (Bieback, K., et al., Transfusion 59:3448-3460, 2019). Additionally, the human platelet lysate can be an acceptable composition procured from a commercial source which has treated the composition as described, or by any other method of producing a commercially acceptable human platelet lysate composition. In some embodiments fresh frozen plasma, or about 2.5% to about 10% human AB serum can be included in the differentiation medium instead of the platelet lysate. In some embodiments, the differentiation culture medium does not include a feeder layer or feeder cells. In some embodiments, the differentiation culture medium is free of fetal bovine serum (FBS), fetal calf serum (FCS), and other animal sourced products and does not include feeder cells or a feeder cell layer.

Although the methods disclosed herein typically and preferably do not use a feeder cell layer, the use of such a feeder cell layer does not alter the advantages of the methods described herein. As used herein, a “feeder cell layer,” “feeder layer,” or “feeder cells” refer to exogenous cells of one type that are co-cultured with cells of a second type (e.g., HSPCs), to provide an environment in which the cells of the second type can be maintained and differentiate or proliferate. Without being bound by any theory, feeder cells can provide, for example, peptides, polypeptides, electrical signals, organic molecules, nucleic acid molecules, growth factors, other factors (e.g., cytokines), and metabolic nutrients to the second type of cells.

In the preferred embodiments of the present methods, the NK cell composition and/or preparation does not contain exogenously added cells, such as exogenous antigen presenting cells (for example, dendritic cells). As used herein, an exogenous cell(s) refers to a cell(s) not derived from the expanded and/or differentiated HSPCs.

The HSPCs are cultured in vitro or ex vivo in the differentiation culture medium for a time period sufficient to produce the NK cell composition and/or preparation. In some embodiments, the time period for differentiation is from about 7 to 21 days, from about 7 to 14 days, from about 12 to 16 days, from about 14 to 16 days, from about 7 days or about 14 days. The time period selected can be dependent upon the temperature used when culturing, the concentration of the cytokines used and other factors.

The status of the differentiating NK cells can be monitored by flow cytometry and the determination of the development of CD56⁺ cells by staining the cells with an anti-CD56 antibody.

The resulting NK cell composition and/or preparation can contain about 50% to about 80% CD56⁺ cells and about 20% to about 50% endogenous CD56⁻ cells. In some embodiments, the NK cell composition and/or preparation comprises from about 50% up to about 85% CD56⁺ cells and about 15% to about 50% CD56⁻, endogenous myeloid-derived cells, such as dendritic cells, macrophages, and granulocytes. In some embodiments, the NK cell composition and/or preparation comprises from about 55% up to about 65% CD56⁺ cells and about 35% to about 45% CD56⁻, endogenous myeloid-derived cells, such as dendritic cells, macrophages, and granulocytes. The CD56⁺ cells are predominantly NK cells. In some embodiments, the NK cell composition and/or preparation comprises from about 70% up to about 85% CD56⁺ cells and about 15% to about 30% CD56⁻, endogenous myeloid-derived cells, such as dendritic cells, macrophages, and granulocytes. In addition to CD56, the CD56⁺ cells express a high frequency of NKp30, NKp46, NKp44, NKG2A, and granzyme B; a moderate to high frequency of perforin and CD107a; a low to moderate frequency of NKG2D; and substantially no KIRs. In some embodiments the CD56⁺ cells are KIR⁻. (As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1.) CD16 is expressed at a low to moderate frequency. In some embodiments, the CD56⁻ cells express a moderate to high frequency of granzyme B; a high frequency of CD107a, and a low frequency of perforin.

After the cells are cultured in the differentiation culture medium for a time sufficient to produce the NK cell composition and/or preparation, the resultant CD56⁺ (mainly NK) cells in the population are typically immature but functional NK cells resembling, but distinct from, NK cells found naturally in the human body. In some embodiments, the CD56⁺ (mainly NK) cells in the NK cell composition and/or preparation are not further differentiated prior to use or storage.

In a certain embodiment, it has been found that priming the HSPCs for NK cell differentiation with IL-15 during the expansion phase increases the number of CD56⁺ cells obtained in the final NK cell composition and/or preparation. Typically, the IL-15 is added during the last about 4 to about 7 days of the expansion phase. Where the expansion phase is about 7 days, priming take place during the last about 4 days. The increase in the total number of CD56⁺ cells using this method can be increased by up to 50% or more and even up to 58%, or more. The remaining steps of the expansion phase and the differentiation phase remain as described above. In this embodiment IL-15 can be added in an amount of about 40 ng/ml to about 100 ng/ml or more.

The NK cells that comprise the compositions and/or preparations produced by any of the above described methods may be genetically engineered to express a molecule or molecules of interest, such as, for example, a protein, nucleic acid, or carbohydrate. In some embodiments, the NK cells comprising the compositions and/or preparations are genetically engineered to express a protein of interest, such as a protein, polypeptide, or peptide (collectively referred to as a protein). In some embodiments, the protein is an antigen recognizing receptor, other cell surface protein(s), or an intracellular molecule. An NK cell of the composition and/or preparation can be genetically modified, prior to, or during the expansion phase and/or during or after the differentiation phase. In a typical embodiment, the NK cells are genetically engineered during the expansion phase. In some embodiments, the NK cells are genetically engineered during or subsequent to the differentiation phase.

The NK cells comprising the compositions and/or preparations can be genetically engineered to express an antigen recognizing receptor(s) that binds to an antigen of interest. In certain embodiments, the antigen recognizing receptor is a chimeric antigen receptor (CAR). In certain embodiments, the antigen recognizing receptor is a T-cell receptor (TCR). The antigen recognizing receptor can bind to, for example, a tumor specific or tumor associated antigen or a pathogen antigen.

In certain embodiments, the antigen recognizing receptor binds to a tumor associated or tumor specific antigen. Any suitable tumor associated or tumor specific antigen (e.g., an antigenic peptide) can be used in the embodiments described herein. Sources of antigen include, but are not limited to, proteins associated with cancer and/or leukemia (e.g., AML) (a tumor associated or tumor specific antigen). An antigen can be expressed as a peptide or as an intact protein or portion thereof. The intact protein or a portion thereof can be native or a variant thereof, such as a mutant form. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13Ralpha2), kappa-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), mesothelin (MSLN), ERBB2, MAGEA3, p53, MART1, gp100, proteinase 3 (PR1), tyrosinase, survivin, hTERT, EphA2, an NKG2D ligand, cancer-testis antigen NY-ESO-1, oncofetal antigen (h5T4), prostate stem cell antigen (PSCA), prostate-specific membrane antigen (PSMA), ROR1, tetraspanin 8 (TSPAN8), tumor-associated glycoprotein 72 (TAG-72), vascular endothelial growth factor R2 (VEGF-R2), Wilms tumor protein (WT-1), cytokine receptor-like factor 2 (CRLF2), BCMA, GPC3, NKCS1, EGF1R, EGFR-VIII, and ERBB.

In some embodiments, the tumor antigen is CD19, ROR1, Her2, PSMA, PSCA, mesothelin (MSLN), or CD20. In some embodiments, the tumor antigen is CD19, CD20, CD33, MSLN, or cytokine receptor-like factor 2 (CRLF2), which are expressed on leukemias or lymphomas.

In some embodiments, the antigen is associated with or specific to leukemia, such as acute lymphocytic leukemia (ALL), acute myelogenous leukemia (AML), chronic lymphocytic leukemia (CLL), or chronic myelogenous leukemia (CML). In a preferred embodiment, the target is associated with or specific to AML. In a preferred embodiment, the antigen is associated with or specific to AML. The antigen can be, but is not limited to, a protein, non-protein, neoantigen, post-translationally modified antigen, peptide-MHC antigen, and/or over-expressed antigen.

In certain embodiments, the AML specific or associated antigen is AMLI-ETO, DEK-CAN, promyelocytic leukemia-retinoic acid receptor a (PML-RARα), Fms-like tyrosine kinase 3-internal tandem duplication (Flt3-ITD), Fms-like tyrosine kinase 3 (Flt3), nucleophosmin 1 (NPM1), Aurora A kinase (AurA), B-cell lymphoma-2 (Bcl-2), Bax inhibitor 1 (B1-1), B lymphoma Mo-MLV insertion region 1 homolog (BMI1), BRCA1-associated protein (BRAP), chronic myeloid leukemia (CML) 28 (CML28), CML66, cyclin B1, cyclin E, cytochrome P450 1B1 (CYP1B1), ETO/MTG8 (myeloid translocation gene on 8q), carbonic anhydrase IX (CAIX), G250/CAIX, homeobox A9 (HOXA9), human telomerase reverse transcriptase (hTERT), myeloid cell leukemia sequence 1 (Mcl-1), mesothelin (MSLN), minor histocompatibility antigen (mHAg) (e.g., lymphoid-restricted histocompatibility antigen 1 (LRH-1)), myeloperoxidase, M-phase phosphoprotein 11 (MPP11), mucin 1 (MUC1), nucleolar and spindle-associated protein 1 (NuSAP1), oncofetal antigen-immature laminin receptor protein (OFA/iLRP), proteinase 3, regulator of G protein signaling 5 (RGS5), receptor for hyaluronic acid-mediated motility (RHAMM), synovial sarcoma X breakpoint 2-interacting protein (SSX2IP), Survivin, Wilms' tumor 1 protein (WT1), cyclin A1, melanoma antigen (MAGE), Per ARNT Sim Domain containing 1 (PASD1), preferentially expressed antigen in melanoma (PRAME), renal antigen-1 (RAGE-1), heat-shock DnaJ protein homolog 2 (HSJ2), myc-associated zinc-finger protein (MAZ), renal cell cancer antigen (NY-REN60), particularly interesting new Cys-His protein (PINCH), recombination signal-binding protein 1 for J-κ (RBPJk), syntaxin, methyl-lysophosphatidic acid (mLPA), α-galactosylceramide (a-GalCer), Lewis Y antigen (LeY), isocitrate dehydrogenase 1 (IDH1 (R132)), isocitrate dehydrogenase 2 (IDH2 (R140)), nucleophosmin 1 mutant (NPM1mut), Notch signaling molecule isoform (Notch variants), hyaluronan receptor isoform (CD44v6), phosphorylated peptides, protein tyrosine phosphatase type IVA member 3 (PRL3), proteinase 3 peptide HLA-A2-restricted (PR1/HLA-A2), 20 Wilms' tumor peptides HLA-A2-restricted (WT1/HLA-A2), interleukin 12 receptor beta 1 (IL12RB1), and/or member of immunoglobulin superfamily (CD96). (Goswami et al., Curr. Drug Targets 18:296-303, 2017).

In certain embodiments, the antigen recognizing receptor binds to a pathogen antigen, e.g., for use in treating and/or preventing a pathogen infection or other infectious disease, for example, in an immunocompromised subject. In certain embodiments, pathogen includes a virus, bacteria, fungi, parasite or protozoa capable of causing disease.

Non-limiting examples of viruses include Retroviridae (e.g., human immunodeficiency viruses, such as HIV-1 and other isolates, such as HIV-LP; Picornaviridae (e.g., polio viruses, hepatitis A virus, enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses, zika virus); Coronoviridae (e.g., coronaviruses, including SARS-CoV and SARS-CoV-2); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Naira viruses); Arena viridae (hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

In certain embodiments, the pathogen antigen is a viral antigen present in Cytomegalovirus (CMV), Epstein Barr Virus (EBV), Human Immunodeficiency Virus (HIV), Herpes simplex virus (HSV1 or HSV2), Hepatitis virus (A, B, or C), zika virus, influenza virus, or coronavirus (SARS-CoV or SARS-CoV-2). In a preferred embodiment the viral antigen is specific to or associated with HIV, HSV 1 or 2, zika virus, Hepatitis A, B, or C, SARS-CoV, or SARS-CoV-2.

Non-limiting examples of bacteria include Pasteurella, Staphylococci, Streptococcus, Escherichia coli, Pseudomonas species, and Salmonella species. Specific examples of infectious bacteria include, but are not limited to, Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococci), Streptococcus agalactiae (Group B Streptococci), Streptococci (viridans group), Streptococcus faecalis, Streptococcus bovis, Streptococci (Anaerobic sps.), Streptococcus pneumoniae, pathogenic Campylobacter sp., Enterococcus sp., Haemophilus influenzae, Bacillus antracis, Corynebacterium diphtherias, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the antigen recognizing receptor is a TCR. A TCR is a disulfide-linked heterodimeric protein consisting of two variable chains expressed as part of a complex with the invariant CD3 chain molecules. A TCR is found on the surface of a T cell and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: a variable (V) region and a constant (C) region. The constant region is proximal to the cell membrane, followed by a transmembrane region and a short cytoplasmic tail. The variable region binds to the peptide/MHC complex. The variable domain of both chains each have three complementarity determining regions (CDRs).

In certain embodiments, a TCR can form a receptor complex with three dimeric signaling modules: CD3delta/epsilon, CD3gamma/epsilon, and CD247zeta/zeta, or zeta/eta. When a TCR complex engages with its antigen and MHC (peptide/MHC), the T cell expressing the TCR complex is activated.

In certain embodiments, NK cells comprising the described compositions and/or preparations are genetically modified to express a recombinant TCR. In certain embodiments, the TCR is a non-naturally occurring TCR. In certain embodiments, the TCR differs from any naturally occurring TCR by at least one amino acid residue. In certain embodiments, the TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the TCR is modified from a naturally occurring TCR by at least one amino acid residue. In certain embodiments, the TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In some embodiments, the TCR specifically binds to an antigen of cytomegalovirus (CMV), adenovirus, human herpesvirus 6 (HHV6), BK virus, Epstein-Barr virus (EBV), HIV, or SARS (SARS-CoV or SARS-CoV-2).

In certain embodiments, the antigen recognizing receptor is a chimeric antigen receptor (CAR). A CAR is an engineered receptor which confers a specificity of interest onto a cell, such as cells of the NK cell compositions and/or preparations described herein. CARs can be used to graft the specificity of a monoclonal antibody onto an NK cell of the NK cell compositions and/or preparations described herein, with transfer of their coding sequence facilitated by, for example, a retroviral vector.

Expanded HSPCs, as described previously, can be differentiated, as described previously, to form CAR-HSPCs and CAR-NK cells. The CAR-HSPCs comprise HSPCs engineered to express the CAR receptor, and which express the engineered CAR receptor. The CAR-NK cells comprise NK cells engineered to express the CAR receptor, and which express the engineered CAR receptor.

Generations of CARs include the following. “First generation” CARs are typically composed of an extracellular antigen binding domain (e.g., a single-chain variable fragment (scFv)) fused to a transmembrane domain, fused to cytoplasmic/intracellular signaling domain of the T cell receptor chain. “First generation” CARs typically have the intracellular signaling domain from the CD3zeta-chain, which is the primary transmitter of signals from endogenous TCRs. “First generation” CARs can provide de novo antigen recognition and cause activation of cells through their CD3zeta chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, or 2B4) to the cytoplasmic tail of the CAR to provide additional signals to the cell. “Second generation” CARs comprise those that provide both co-stimulation (e.g., CD28, 4-1BB or 2B4) and activation (CD3zeta). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3zeta).

In some embodiments, a co-stimulatory domain can be CD27, CD28, 4-1BB (CD137), OX40 (CD134), CD30, CD40, lymphocyte function-associated antigen-1 (LFA-1), CD2, CD7, LIGHT, NKG2C, and/or B7-H3 costimulatory domains. In some embodiments, a co-stimulatory domain can be CD27, CD28, 4-1BB (CD137), OX40 (CD134), DAP10, DAP12, ICOS, and/or 2B4. In some embodiments, a co-stimulatory domain can be CD27, CD28, 4-1BB, 2B4, DAP10, DAP12, OX40, CD30, CD40, lymphocyte function-associated antigen-1 (LFA-1), CD2, CD7, LIGHT, NKG2C, and/or B7-H3 costimulatory domains. In some embodiments, an intracellular signaling domain can be a domain of CD3zeta, CD28 and/or 4-1BB.

In certain non-limiting embodiments, the extracellular antigen-binding domain of the CAR (embodied, for example, as an scFv or an analog thereof) binds to an antigen with a dissociation constant (K_{d}) of about 2×10⁻⁷ M or less. In certain embodiments, the K_{d} is about 1×10⁻⁷ M or less, about 5×10⁻⁸ M or less, about 1×10⁻⁸ M or less, about 5×10⁻⁹ M or less, or about 1×10⁻⁹ M or less.

Binding of an extracellular antigen-binding domain (for example, an scFv or an analog thereof) of an antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

A CAR can comprise an extracellular antigen-binding domain, a transmembrane domain and an intracellular signaling domain, wherein the extracellular antigen-binding domain specifically binds to an antigen, e.g., a tumor antigen or a pathogen antigen, including for example a viral or bacterial antigen.

In certain embodiments, the extracellular antigen-binding domain specifically binds to an antigen. In certain embodiments, the extracellular antigen-binding domain is an scFv. In certain embodiments, the scFv is a human scFv. In certain embodiments, the scFv is a humanized scFv. In certain embodiments, the extracellular antigen-binding domain is a Fab, which is optionally crosslinked. In certain embodiments, the extracellular binding domain is a F(ab′)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen, including for example, a viral or a bacterial antigen.

In certain embodiments, the extracellular binding domain is an scFv that specifically binds to CD19, such as an scFv derived from the FMC63 antibody or 4G7 antibody. In some embodiments, the scFv comprises the CDRs of the FMC63 antibody: a CDRL1 sequence of RASQDISKYLN (SEQ ID NO:1), a CDRL2 sequence of SRLHSGV (SEQ ID NO:2), a CDRL3 sequence of GNTLPYTFG (SEQ ID NO: 3), a CDRH1 sequence of DYGVS (SEQ ID NO:4), a CDRH2 sequence of VTWGSETTYYNSALKS (SEQ ID NO:5), and a CDRH3 sequence of YAMDYWG (SEQ ID NO:6); or a CDRL1 sequence of RASQDISKYLN (SEQ ID NO:1), a CDRL2 sequence of SRLHSGV (SEQ ID NO:2), a CDRL3 sequence of GNTLPYTFG (SEQ ID NO:3), a CDRH1 sequence of DYGVS (SEQ ID NO:4), a CDRH2 sequence of DNSKSQ (SEQ ID NO:63), and a CDRH3 sequence of YAMDYWG (SEQ ID NO:6). In some embodiments, an extracellular binding domain is an scFv derived from or comprising the heavy and light chain variable regions of antibody FMC63. The heavy and light chain variable regions of antibody FMC63 are shown in SEQ ID NO:64 and SEQ ID NO:65, respectively.

FMC63 heavy chain variable region
SEQ ID NO: 64
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSS
FMC63 light chain variable region
SEQ ID NO: 65
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT

In some embodiments, an scFV comprises the heavy and light chain CDRs of the CD19 monoclonal antibody 4G7, as set forth in SEQ ID NO:25 and SEQ ID NO: 26 or SEQ ID NO:27. In some embodiments, the extracellular antigen binding comprises the heavy and light chain variable region CDRs set forth in SEQ ID NO:25 and 26, or in SEQ NO: 25 and 27. In some embodiments, an scFV is derived from the CD19 monoclonal antibody 4G7, preferably comprises a part of the binding domains of CD19 monoclonal antibody 4G7, portions of variable region of the CD19 monoclonal antibody 4G7 immunoglobulin gamma 1 heavy chain (SEQ ID NO:25) and the variable fragments of the CD19 monoclonal antibody 4G7 immunoglobulin kappa light chain (SEQ ID NO:26 or SEQ ID NO:27) linked together by a flexible linker. In a particular embodiment, the flexible linker has the amino acid sequence set forth in SEQ ID NO:28. In some embodiments, the extracellular antigen binding comprises the heavy and light chain variable region CDRs set forth in SEQ ID NO:25 and SEQ ID NO:26, or in SEQ NO: 25 and SEQ ID NO:27.

anti human CD 19 monoclonal antibody
4G7 heavy chain variable region:
SEQ ID NO: 25
EVQLQQSGPELIKPGASVKMSCKASGYTFTSYVMHWVKQKPGQGLEWIGY
1NPYNDGTKYNEKFKGKATLTSDKSSSTAYMELSSLTSEDSAVYYCARGT
YYYGSRVFDYWGQGTTLTVSS
anti-human CD19 monoclonal antibody 4G7
immunoglobulin kappa light chain variable region:
SEQ ID NO: 26
DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRAD
anti-human CD19 monoclonal antibody
4G7 immunoglobulin kappa light chain variable
region:
SEQ ID NO: 27
DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRSDP
flexible linker:
SEQ ID NO: 28
GGGGSGGGGSGGGGS

In some embodiments, an scFV is derived from a CLA (cutaneous lymphocyte antigen) monoclonal antibody and comprises the heavy and light chain variable region of the CLA monoclonal antibody set forth in SEQ ID NO:29 and SEQ ID NO: 30, respectively (see US Patent Application Publication No. 2019/0209611; incorporated herein by reference). In some embodiments, the extracellular antigen binding domain comprises the heavy and light chain variable region CDRs set forth in SEQ ID NO:29 and SEQ ID NO:30.

CLA VH (heavy chain variable region)
SEQ ID NO: 29
EVQLVESGGGLVQPGNSLKLSCSASGFTFSSYGMHWIRQAPGEGLDWVAY
ISSSSGTVYADAVKARFTISRDNAKNTLYLQLNSLKSEDTAIYYCARAQN
WDLFDYWGQGVMVTVSS
CLA VL (light chain variable region)
SEQ ID NO: 30
QIMLTQQAESLWISPGERVSITCRASQSLLYTDGKHYLSWYQQKPGQTTK
ALIYHASVRTDGVPTRFIGSGSGTEFTLSIEHVQPEDFAIYYCLQTLKSP
FTFGSGTKLEIK

In some embodiments, an scFV is derived from a CD142 monoclonal antibody and comprises the heavy and light chain variable region of the CD142 monoclonal antibody set forth in SEQ ID NOs: 31 and 32, respectively (see US Patent Application Publication No. 2019/0209611, incorporated herein by reference). In some embodiments, the extracellular antigen binding domain comprises the heavy and light chain variable region CDRs set forth in SEQ ID NO:31 and SEQ ID NO:32.

CD142 VH (heavy chain variable region)
SEQ ID NO: 31
QVQLKQSGPGLVQPSQSLSITCTVSGFSLSNYGVHWVRQSPGKGLEWLGV
IWSGGSTDYNVAFISRLIITKDNSKSQVFLKMNSLQADDTAIYFCARTTG
SVFNAMDHWGQGTSVTVSS
CD142 VL (light chain variable region)
SEQ ID NO: 32
QIVLTQSPALMSASPGEKVTMTCSASSSVTYMYWYQQKPRSSPKPWIYLT
SNLASGVPARFSGSGSGTSYSLTISSVEAEDAATYYCQQWSSNPLTFGAG
TKLELK

In some embodiments, an scFV is derived from a CD73 monoclonal antibody and comprises the heavy and light chain variable region of the CD73 monoclonal antibody set forth in SEQ ID NOs: 33 and 34, respectively (see US Patent Application Publication No. 2019/0209611, incorporated herein by reference). In some embodiments, the extracellular antigen binding domain comprises the heavy and light chain variable region CDRs set forth in SEQ ID NO:33 and SEQ ID NO:34.

CD73 VH (heavy chain variable region)
SEQ ID NO: 33
EVQLQQSGAELVKPGASVKLSCTASGFNIKDTYIHWVKQRPEQGLEWIGR
IDPATGNTEYDPKFQGKATITADTSSNTAYLHLSSLTSEDTAVYYCARGY
YGSSYPPWFAYWGQGTLVTVSA
CD73 VL (light chain variable region)
SEQ ID NO: 34
DIVMTQSHKFMSTSVGDRVSITCKASQDVGSAVAWYQQKPGQSPKLLIYW
ASTRHTGVPDRFTGSGSGTDFTLTISNVQSEDLADYFCQQYSSYPLTFGA
GTKLELK

In some embodiments, an scFV is derived from a CD49c monoclonal antibody and comprises the heavy and light chain variable region of the CD49c monoclonal antibody set forth in SEQ ID NOs: 35 and 36, respectively (see US Patent Application Publication No. 2019/0209611; incorporated herein by reference). In some embodiments, the extracellular antigen binding domain comprises the heavy and light chain variable region CDRs set forth in SEQ ID NO:35 and SEQ ID NO:36.

CD49c VH (heavy chain variable region)
SEQ ID NO: 35
EVQLQQSGAELVKPGASVKLSCTASGFNIKDTYMHWVKQRPEQGLEWIGR
IDPANGHTKYDPKFQGKATITADTSSNAAYLQLNSLTSEDTAVYYCARRV
AYAMDYWGQGTSVTVSS
CD49c VL (light chain variable region)
SEQ ID NO: 36
ENVLTQSPAIMSASPGEKVTMTCSASSSVTYMHWYQQKSSTSPKLWIYDT
SKLASGVPGRFSGSGSGNSYSLTISSMEAEDVATYCCFQGSGYPLTFGGG
TKLEIK

In some embodiments, an scFV is derived from a CD66c monoclonal antibody and comprises the heavy and light chain variable region of the CD66c monoclonal antibody set forth in SEQ ID NOs: 37 and 38, respectively (see US Patent Application Publication No. 2019/0209611, incorporated herein by reference). In some embodiments, the extracellular antigen binding domain comprises the heavy and light chain variable region CDRs set forth in SEQ ID NO:37 and SEQ ID NO:38.

CD66c VH (heavy chain variable region)
SEQ ID NO: 37
QVTLKESGPGILKPSQTLSLTCSFSGFSLSTSGMGVGWIRQPSGKSLEWL
AHIWWNDERYYNPSLKNQLTISKDTSRNQVFLKITSVDTADTATYYCARS
PRGYFDYWGHGTTLTVSS
CD66c VL (light chain variable region)
SEQ ID NO: 38
DIVMTQSQKFMSTSVGDRVSVTCKASQNVVTNVAWYQQTPGQSPKALIYS
ASYRYSGVPDRFSGSGSGTDFTLTISNVQSGDLAEYFCQQYNSYPLTFGA
GTKLELK

In some embodiments, an scFV is derived from a CD104 monoclonal antibody and comprises the heavy and light chain variable region of the CD104 monoclonal antibody set forth in SEQ ID NOs: 39 and 40, respectively (see US Patent Application Publication No. 2019/0209611; incorporated herein by reference). In some embodiments, the extracellular antigen binding domain comprises the heavy and light chain variable region CDRs set forth in SEQ ID NO:39 and SEQ ID NO:40.

CD104 VH (heavy chain variable region)
SEQ ID NO: 39
QVNLLQSGAALVKPGASVKLSCKASGYTFTDYYIFWVKQSHGKSLEWIGY
INPNSGSTNYNEKFKRKATLSVDKSTNTAYMELSRLTSEDSATYYCTRRA
YYGYNPFDYWGQGVMVTVSS
CD104 VL (light chain variable region)
SEQ ID NO: 40
DIQMTQTPSSMPASLGERVTISCRASRGINNYLSWYQQNLDGTIKPLIYY
TSNLQSGVPSRFSGSGSGTDYSLTISSLEPEDFAMYYCQQYDSSPWTFGG
GTKLELK

In some embodiments, an scFV is derived from a CD318 monoclonal antibody and comprises the heavy and light chain variable region of the CD318 monoclonal antibody set forth in SEQ ID NO:41 and SEQ ID NO:42, respectively (see US Patent Application Publication No. 2019/0209611; incorporated herein by reference). In some embodiments, the extracellular antigen binding domain comprises the heavy and light chain variable region CDRs set forth in SEQ ID NO:41 and SEQ ID NO:42.

CD318 VH (heavy chain variable region)
SEQ ID NO: 41
EVQLQQSGAELVRPGALVKLSCKASGFNIKDYYIHWVKQRPEQGLEWIGW
IDPENGHTIYDPKFQGKASITADTSSNTAYLQLSSLTSEDTAVYYCARLT
GTTYAMDYWGQGTSVTVSS
CD318 VL (light chain variable region)
SEQ ID NO: 42
DIVMTQSHKFMSTSVGDRVSITCKASQDVSTAVAWYQQKSGQSPKLLIYW
ASTRHTGVPDRFTGSGSGTDYTLTISSVQAEDLALYYCQQHYSTPYTFGG
GTKLEIK

In some embodiments, an scFV is derived from a TSPAN8 monoclonal antibody and comprises the heavy and light chain variable region of the TSPAN8 monoclonal antibody set forth in SEQ ID NOs: 43 and 44, respectively (see US Patent Application Publication No. 2019/0209611; incorporated herein by reference). In some embodiments, the extracellular antigen binding domain comprises the heavy and light chain variable region CDRs set forth in SEQ ID NO:43 and SEQ ID NO:44.

TSPAN8 VH (heavy chain variable region)
SEQ ID NO: 43
EVKLLESGGGLVQPGGSMRLSCAASGFTFTDFYMNWIRQPAGKAPEWLGF
IRNKASGYTTEYNPSVKGRFTISRDNTQNMLYLQMNTLRAEDTATYYCAR
AHSYYGYDYFDYWGQGVMVTVSS
TSPAN8 VL (light chain variable region)
SEQ ID NO: 44
DIQMTQSPASLSASLEEIVTITCQASQDIGNWLSWYQQKPGKSPQLLIYG
ATSLADGVPSRFSGSRSGTQYSLKISRLQVEDIRIYYCLQAYSAPWTFGG
GTKLELK

In some embodiments, an scFV to CLA has the amino acid sequence set forth in SEQ ID NO:45 or SEQ ID NO:46 (see US Patent Application Publication No. 2019/0209611; incorporated herein by reference).

CLA specific scFv VH-linker-VL
SEQ ID NO: 45
EVQLVESGGGLVQPGNSLKLSCSASGFTFSSYGMHWIRQAPGEGLDWVAY
ISSSSGTVYADAVKARFTISRDNAKNTLYLQLNSLKSEDTAIYYCARAQN
WDLFDYWGQGVMVTVSSGGGGSGGGGSGGGGSQIMLTQQAESLWISPGER
VSITCRASQSLLYTDGKHYLSWYQQKPGQTTKALIYHASVRTDGVPTRFI
GSGSGTEFTLSIEHVQPEDFAIYYCLQTLKSPFTFGSGTKLEIK
CLA specific scFv VL-linker-VH
SEQ ID NO: 46
QIMLTQQAESLWISPGERVSITCRASQSLLYTDGKHYLSWYQQKPGQTTK
ALIYHASVRTDGVPTRFIGSGSGTEFTLSIEHVQPEDFAIYYCLQTLKSP
FTFGSGTKLEIKGGGGSGGGGSGGGGSEVQLVESGGGLVQPGNSLKLSCS
ASGFTFSSYGMHWIRQAPGEGLDWVAYISSSSGTVYADAVKARFTISRDN
AKNTLYLQLNSLKSEDTAIYYCARAQNWDLFDYWGQGVMVTVSS

In some embodiments, an scFV to CD142 has the amino acid sequence set forth in SEQ ID NO:47 or SEQ ID NO:48 (see US Patent Application Publication No. 2019/0209611; incorporated herein by reference).

CD142 specific CAR sequence VH-linker-VL
SEQ ID NO: 47
QVQLKQSGPGLVQPSQSLSITCTVSGFSLSNYGVHWVRQSPGKGLEWLGV
IWSGGSTDYNVAFISRLIITKDNSKSQVFLKMNSLQADDTAIYFCARTTG
SVFNAMDHWGQGTSVTVSSGGGGSGGGGSGGGGSQIVLTQSPALMSASPG
EKVTMTCSASSSVTYMYWYQQKPRSSPKPWIYLTSNLASGVPARFSGSGS
GTSYSLTISSVEAEDAATYYCQQWSSNPLTFGAGTKLELK
CD142 specific CAR sequence VL-linker-VH
SEQ ID NO: 48
QIVLTQSPALMSASPGEKVTMTCSASSSVTYMYWYQQKPRSSPKPWIYLT
SNLASGVPARFSGSGSGTSYSLTISSVEAEDAATYYCQQWSSNPLTFGAG
TKLELKGGGGSGGGGSGGGGSQVQLKQSGPGLVQPSQSLSITCTVSGFSL
SNYGVHWVRQSPGKGLEWLGVIWSGGSTDYNVAFISRLIITKDNSKSQVF
LKMNSLQADDTAIYFCARTTGSVFNAMDHWGQGTSVTVSS

In some embodiments, an scFV to CD73 has the amino acid sequence set forth in SEQ ID NO:49 or SEQ ID NO:50 (see US Patent Application Publication No. 2019/0209611; incorporated herein by reference).

CD73 specific CAR sequence VH-linker-VL
SEQ ID NO: 49
EVQLQQSGAELVKPGASVKLSCTASGFNIKDTYIHWVKQRPEQGLEWIGR
IDPATGNTEYDPKFQGKATITADTSSNTAYLHLSSLTSEDTAVYYCARGY
YGSSYPPWFAYWGQGTLVTVSAGGGGSGGGGSGGGGSDIVMTQSHKFMST
SVGDRVSITCKASQDVGSAVAWYQQKPGQSPKLLIYWASTRHTGVPDRFT
GSGSGTDFTLTISNVQSEDLADYFCQQYSSYPLTFGAGTKLELK
CD73 specific CAR sequence VL-linker-VH
SEQ ID NO: 50
DIVMTQSHKFMSTSVGDRVSITCKASQDVGSAVAWYQQKPGQSPKLLIYW
ASTRHTGVPDRFTGSGSGTDFTLTISNVQSEDLADYFCQQYSSYPLTFGA
GTKLELKGGGGSGGGGSGGGGSEVQLQQSGAELVKPGASVKLSCTASGFN
IKDTYIHWVKQRPEQGLEWIGRIDPATGNTEYDPKFQGKATITADTSSNT
AYLHLSSLTSEDTAVYYCARGYYGSSYPPWFAYWGQGTLVTVSA

In some embodiments, an scFV to CD49c has the amino acid sequence set forth in SEQ ID NO:51 or SEQ ID NO:52 (see US Patent Application Publication No. 2019/0209611; incorporated herein by reference).

CD49c specific CAR sequence VH-linker-VL
SEQ ID NO: 51
EVQLQQSGAELVKPGASVKLSCTASGFNIKDTYMHWVKQRPEQGLEWIGR
IDPANGHTKYDPKFQGKATITADTSSNAAYLQLNSLTSEDTAVYYCARRV
AYAMDYWGQGTSVTVSSGGGGSGGGGSGGGGSENVLTQSPAIMSASPGEK
VTMTCSASSSVTYMHWYQQKSSTSPKLWIYDTSKLASGVPGRFSGSGSGN
SYSLTISSMEAEDVATYCCFQGSGYPLTFGGGTKLEIK
CD49c specific CAR sequence VL-linker-VH
SEQ ID NO: 52
ENVLTQSPAIMSASPGEKVTMTCSASSSVTYMHWYQQKSSTSPKLWIYDT
SKLASGVPGRFSGSGSGNSYSLTISSMEAEDVATYCCFQGSGYPLTFGGG
TKLEIKGGGGSGGGGSGGGGSEVQLQQSGAELVKPGASVKLSCTASGFNI
KDTYMHWVKQRPEQGLEWIGRIDPANGHTKYDPKFQGKATITADTSSNAA
YLQLNSLTSEDTAVYYCARRVAYAMDYWGQGTSVTVSS

In some embodiments, an scFV to CD66c has the amino acid sequence set forth in SEQ ID NO:53 or SEQ ID NO:54 (see US Patent Application Publication No. 2019/0209611; incorporated herein by reference).

CD66c specific CAR sequence VH-linker-VL
SEQ ID NO: 53
QVTLKESGPGILKPSQTLSLTCSFSGFSLSTSGMGVGWIRQPSGKSLEWL
AHIWWNDERYYNPSLKNQLTISKDTSRNQVFLKITSVDTADTATYYCARS
PRGYFDYWGHGTTLTVSSGGGGSGGGGSGGGGSDIVMTQSQKFMSTSVGD
RVSVTCKASQNVVTNVAWYQQTPGQSPKALIYSASYRYSGVPDRFSGSGS
GTDFTLTISNVQSGDLAEYFCQQYNSYPLTFGAGTKLELK
CD66c specific CAR sequence VL-linker-VH
SEQ ID NO: 54
DIVMTQSQKFMSTSVGDRVSVTCKASQNVVTNVAWYQQTPGQSPKALIYS
ASYRYSGVPDRFSGSGSGTDFTLTISNVQSGDLAEYFCQQYNSYPLTFGA
GTKLELKGGGGSGGGGSGGGGSQVTLKESGPGILKPSQTLSLTCSFSGFS
LSTSGMGVGWIRQPSGKSLEWLAHIWWNDERYYNPSLKNQLTISKDTSRN
QVFLKITSVDTADTATYYCARSPRGYFDYWGHGTTLTVSS

In some embodiments, an scFV to CD104 has the amino acid sequence set forth in SEQ ID NO:55 or SEQ ID NO:56 (see US Patent Application Publication No. 2019/0209611; incorporated herein by reference).

CD104 specific CAR sequence VH-linker-VL
SEQ ID NO: 55
QVNLLQSGAALVKPGASVKLSCKASGYTFTDYYIFWVKQSHGKSLEWIGY
INPNSGSTNYNEKFKRKATLSVDKSTNTAYMELSRLTSEDSATYYCTRRA
YYGYNPFDYWGQGVMVTVSSGGGGSGGGGSGGGGSDIQMTQTPSSMPASL
GERVTISCRASRGINNYLSWYQQNLDGTIKPLIYYTSNLQSGVPSRFSGS
GSGTDYSLTISSLEPEDFAMYYCQQYDSSPWTFGGGTKLELK
CD104 specific CAR sequence VL-linker-VH
SEQ ID NO: 56
DIQMTQTPSSMPASLGERVTISCRASRGINNYLSWYQQNLDGTIKPLIYY
TSNLQSGVPSRFSGSGSGTDYSLTISSLEPEDFAMYYCQQYDSSPWTFGG
GTKLELKGGGGSGGGGSGGGGSQVNLLQSGAALVKPGASVKLSCKASGYT
FTDYYIFWVKQSHGKSLEWIGYINPNSGSTNYNEKFKRKATLSVDKSTNT
AYMELSRLTSEDSATYYCTRRAYYGYNPFDYWGQGVMVTVSS

In some embodiments, an scFV to CD318 has the amino acid sequence set forth in SEQ ID NO:57 or SEQ ID NO:58 (see US Patent Application Publication No. 2019/0209611; incorporated herein by reference).

CD318 specific CAR sequence VH-linker-VL
SEQ ID NO: 57
EVQLQQSGAELVRPGALVKLSCKASGFNIKDYYIHWVKQRPEQGLEWIGW
IDPENGHTIYDPKFQGKASITADTSSNTAYLQLSSLTSEDTAVYYCARLT
GTTYAMDYWGQGTSVTVSSGGGGSGGGGSGGGGSDIVMTQSHKFMSTSVG
DRVSITCKASQDVSTAVAWYQQKSGQSPKLLIYWASTRHTGVPDRFTGSG
SGTDYTLTISSVQAEDLALYYCQQHYSTPYTFGGGTKLEIK
CD318 specific CAR sequence VL-linker-VH
SEQ ID NO: 58
DIVMTQSHKFMSTSVGDRVSITCKASQDVSTAVAWYQQKSGQSPKLLIYW
ASTRHTGVPDRFTGSGSGTDYTLTISSVQAEDLALYYCQQHYSTPYTFGG
GTKLEIKGGGGSGGGGSGGGGSEVQLQQSGAELVRPGALVKLSCKASGFN
IKDYYIHWVKQRPEQGLEWIGWIDPENGHTIYDPKFQGKASITADTSSNT
AYLQLSSLTSEDTAVYYCARLTGTTYAMDYWGQGTSVTVSS

In some embodiments, an scFV to TSPAN8 has the amino acid sequence set forth in SEQ ID NO:59 or SEQ ID NO:60 (see US Patent Application Publication No. 2019/0209611; incorporated herein by reference).

TSPAN8 specific CAR sequence VH-linker-VL
SEQ ID NO: 59
EVKLLESGGGLVQPGGSMRLSCAASGFTFTDFYMNWIRQPAGKAPEWLGF
IRNKASGYTTEYNPSVKGRFTISRDNTQNMLYLQMNTLRAEDTATYYCAR
AHSYYGYDYFDYWGQGVMVTVSSGGGGSGGGGSGGGGSDIQMTQSPASLS
ASLEEIVTITCQASQDIGNWLSWYQQKPGKSPQLLIYGATSLADGVPSRF
SGSRSGTQYSLKISRLQVEDIRIYYCLQAYSAPWTFGGGTKLELK
TSPAN8 specific CAR sequence VL-linker-VH
SEQ ID NO: 60
DIQMTQSPASLSASLEEIVTITCQASQDIGNWLSWYQQKPGKSPQLLIYG
ATSLADGVPSRFSGSRSGTQYSLKISRLQVEDIRIYYCLQAYSAPWTFGG
GTKLELKGGGGSGGGGSGGGGSEVKLLESGGGLVQPGGSMRLSCAASGFT
FTDFYMNWIRQPAGKAPEWLGFIRNKASGYTTEYNPSVKGRFTISRDNTQ
NMLYLQMNTLRAEDTATYYCARAHSYYGYDYFDYWGQGVMVTVSS

### Transmembrane Domain of a CAR

In some embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. The transmembrane domain of the CAR can comprise, for example, a CD8 polypeptide, a CD28 polypeptide, a CD3zeta polypeptide, a CD4 polypeptide, a 4-1BB polypeptide, an OX40 polypeptide, an ICOS polypeptide, a NKG2D polypeptide, a synthetic peptide (not based on a protein associated with the immune response), or a combination thereof.

In certain embodiments, the transmembrane domain comprises a CD8 polypeptide. In certain embodiments, the CD8 polypeptide has an amino acid sequence that is at least about 85%, about 90%, about 95%, about 96%, about 97%, about 98%, about 99% or about 100% identical to the sequence having a NCBI Reference No: NP_001139345.1, SEQ ID NO:7, (sequence identity herein may be determined using standard software such as BLAST or FASTA), or fragments thereof, and/or may optionally comprise up to one or up to two or up to three conservative amino acid substitutions. As used herein, a “conservative amino acid substitution” means an amino acid substitution wherein an amino acid is substituted for another electronically similar amino acid. For example, an amino acid with a hydrophobic side chain can be substituted for a different amino acid also having a hydrophobic side chain (e.g., leucine substituted for isoleucine, alanine substituted for valine, and the like); an amino acid with an acidic chain can be substituted for a different amino acid also having an acidic side chain (e.g., aspartic acid substituted for glutamic acid, and the like); an amino acid with a basic chain can be substituted for a different amino acid also having a basic side chain (e.g., lysine substituted for arginine, and the like); and an amino acid with a polar side chain can be substituted for a different amino acid also having a polar side chain (e.g., serine substituted for threonine, and the like). In certain embodiments, the CD8 polypeptide can have an amino acid sequence that is a consecutive portion of SEQ ID NO:7 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO:7. In certain embodiments, a CAR comprises a transmembrane domain comprising a human CD8 polypeptide that comprises an amino acid sequence of amino acids 137 to 209 of SEQ ID NO:7.

SEQ ID NO: 7:
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the CD8 polypeptide has an amino acid sequence that is at least about 85%, about 90%, about 95%, about 96%, about 97%, about 98%, about 99% or about 100% identical to the sequence having a NCBI Reference No: AAA92533.1, SEQ ID NO:8, or fragments thereof, and/or may optionally comprise up to one or up to two or up to three conservative amino acid substitutions. In certain embodiments, the CD8 polypeptide can have an amino acid sequence that is a consecutive portion of SEQ ID NO:8 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO:8. In certain embodiments, the CAR comprises a transmembrane domain comprising a murine CD8 polypeptide that comprises an amino acid sequence of amino acids 151 to 219 of SEQ ID NO:8.

SEQ ID NO: 8:
MASPLTRELSLNLLLMGESIILGSGEAKPQAPELRIFPKKMDAELGQKVD
LVCEVLGSVSQGCSWLFQNSSSKLPQPTFVVYMASSHNKITWDEKLNSSK
LFSAVRDTNNKYVLTLNKFSKENEGYYFCSVISNSVMYFSSVVPVLQKVN
STTTKPVLRTPSPVHPTGTSQPQRPEDCRPRGSVKGTGLDFACDIYIWAP
LAGICVAPLLSLIITLICYHRSRKRVCKCPRPLVRQEGKPRPSEKIV

In certain embodiments, the CD8 polypeptide comprises or has the amino acid sequence set forth in SEQ ID NO:9.

SEQ ID NO: 9:
STTTKPVLRTPSPVHPTGTSQPQRPEDCRPRGSVKGTGLDFACDIYIWAP
LAGICVALLLSLIITLICY

In certain embodiments, the transmembrane domain of a CAR comprises a CD28 polypeptide. The CD28 polypeptide can have an amino acid sequence that is at least about 85%, about 90%, about 95%, about 96%, about 97%, about 98%, about 99% or 100% identical to the sequence having a NCBI Reference No: P10747 or NP_006130 (SEQ ID No:10), or fragments thereof, and/or may optionally comprise up to one or up to two or up to three conservative amino acid substitutions. In non-limiting certain embodiments, the CD28 polypeptide can have an amino acid sequence that is a consecutive portion of SEQ ID NO:10 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO:10. In certain embodiments, the CD28 polypeptide comprised in the transmembrane domain of a presently disclosed CAR has an amino acid sequence of amino acids 153 to 179 of SEQ ID NO:10.

SEQ ID NO: 10:
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the transmembrane domain of a CAR comprises a NKG2D polypeptide. The NKG2D polypeptide can have an amino acid sequence that is at least about 85%, about 90%, about 95%, about 96%, about 97%, about 98%, about 99% or 100% identical to the sequence having a NCBI Reference No: NP_0031386.2 (SEQ ID NO:66), or fragments thereof, and/or may optionally comprise up to one or up to two or up to three conservative amino acid substitutions. In non-limiting certain embodiments, the NKG2D polypeptide can have an amino acid sequence that is a consecutive portion of SEQ ID NO:66 which is at least 20, or at least 30, or at least 40, or at least 50, and up to 216 amino acids in length. In certain embodiments, the NKG2D polypeptide comprises the transmembrane domain of SEQ ID NO: 10 (e.g., amino acids 52-72).

SEQ ID NO: 66:
MGWIRGRRSRHSWEMSEFHNYNLDLKKSDFSTRWQKQRCPVVKSKCRENA
SPHFCCFIAVAMGIRFIIMVTIWSAVFLNSLFNQEVQIPLTESYCGPCP
KNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYI
ENCSTPNTYICMQRTV

In some embodiments, the transmembrane domain of a CAR comprises a CD3zeta (CD3C) polypeptide. The transmembrane domain of the CD3zeta polypeptide can have an amino acid sequence that is at least about 85%, about 90%, about 95%, about 96%, about 97%, about 98%, about 99% or 100% identical to the sequence of the transmembrane domain of CD3zeta set forth in NCBI Reference No: NP_932170 (SEQ ID NO:11), SEQ ID NO:12, or SEQ ID NO:13, and/or may optionally comprise up to one or up to two or up to three conservative amino acid substitutions.

In some embodiments, the transmembrane domain of a CAR comprises a CD4 polypeptide. The transmembrane domain of the CD4 polypeptide can have an amino acid sequence that is at least about 85%, about 90%, about 95%, about 96%, about 97%, about 98%, about 99% or 100% identical to the sequence of the transmembrane domain of CD4 set forth in NCBI Reference No: NP_000607.1, NP 001181943.1 or NP_001181946.1, (each incorporated herein by reference) and/or may optionally comprise up to one or up to two or up to three conservative amino acid substitutions.

In some embodiments, the transmembrane domain of a CAR comprises a 4-1BB polypeptide. The transmembrane domain of the 4-1BB polypeptide can have an amino acid sequence that is at least about 85%, about 90%, about 95%, about 96%, about 97%, about 98%, about 99% or 100% identical to the sequence of the transmembrane domain of 4-1BB set forth in NCBI Reference No: P41273 (incorporated herein by reference) or NP_001552.2 (SEQ ID NO:18), and/or may optionally comprise up to one or up to two or up to three conservative amino acid substitutions.

In some embodiments, the transmembrane domain of a CAR comprises an OX40 polypeptide. The transmembrane domain of the OX40 polypeptide can have an amino acid sequence that is at least about 85%, about 90%, about 95%, about 96%, about 97%, about 98%, about 99% or 100% identical to the sequence of the transmembrane domain of OX40 set forth in NCBI Reference No: NP_003318.1 (incorporated herein by reference), and/or may optionally comprise up to one or up to two or up to three conservative amino acid substitutions.

In some embodiments, the transmembrane domain of a CAR comprises an ICOS polypeptide. The transmembrane domain of the ICOS polypeptide can have an amino acid sequence that is at least about 85%, about 90%, about 95%, about 96%, about 97%, about 98%, about 99% or 100% identical to the sequence of the transmembrane domain of ICOS set forth in NCBI Reference No: NP_036224.1 (incorporated herein by reference), and/or may optionally comprise up to one or up to two or up to three conservative amino acid substitutions. Spacer Region

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG₁ (GenPept Ref No.: P01857.1, incorporated herein by reference), or the CH₂CH₃ region of an immunoglobulin (e.g., IgG₄ (GenPept Ref No.: P01861.1, incorporated herein by reference) and portions of CD3, a portion of a CD28 polypeptide (e.g., a portion of SEQ ID NO:10), a portion of a CD8 polypeptide (e.g., a portion of SEQ ID NO:7, or a portion of SEQ ID NO:8), a variation of any of the foregoing which is at least about 80%, at least about 85%, at least about 90%, or at least about 95% identical thereto, or a synthetic spacer sequence.

### Intracellular Signaling Domain of a CAR

In certain non-limiting embodiments, an intracellular signaling domain of the CAR can comprise a CD3zeta (CD3C) polypeptide, which can activate or stimulate a cell (e.g., a cell of the lymphoid lineage, e.g., an NK cell). CD35 comprises 3 immunoreceptor tyrosine-based activation motifs (ITAMs), and transmits an activation signal to the cell (e.g., a cell of the lymphoid lineage, e.g., an NK cell) after antigen is bound. In certain embodiments, the CD3zeta polypeptide has an amino acid sequence that is at least about 85%, about 90%, about 95%, about 96%, about 97%, about 98%, about 99% or about 100% identical to the sequence having a NCBI Reference No: NP_932170 (SEQ ID NO:11), or fragments thereof, and/or can optionally comprise up to one or up to two or up to three conservative amino acid substitutions. In certain non-limiting embodiments, the CD32 polypeptide comprises or has an amino acid sequence that is a consecutive portion of SEQ ID NO: 11, which is at least 20, or at least 30, or at least 40, or at least 50, and up to 164 amino acids in length. Alternatively or additionally, in non-limiting various embodiments, the CD3zeta polypeptide comprises or has an amino acid sequence of amino acids 1 to 164, 1 to 50, 50 to 100, 100 to 150, or 150 to 164 of SEQ ID NO:11. In certain embodiments, the CD3 polypeptide comprises or has an amino acid sequence of amino acids 52 to 164 of SEQ ID NO:11.

SEQ ID NO: 11:
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3zeta polypeptide has an amino acid sequence that is at least about 85%, about 90%, about 95%, about 96%, about 97%, about 98%, about 99% or about 100% identical to the sequence having a NCBI Reference No: NP_001106864.2 (SEQ ID NO:12), or fragments thereof, and/or may optionally comprise up to one or up to two or up to three conservative amino acid substitutions. In certain non-limiting embodiments, the CD3zeta polypeptide can have an amino acid sequence that is a consecutive portion of SEQ ID NO:12, which is at least about 20, or at least about 30, or at least about 40, or at least about 50, or at least about 90, or at least about 100, and up to 188 amino acids in length. Alternatively or additionally, in non-limiting various embodiments, the CD32 polypeptide comprises or has an amino acid sequence of amino acids 1 to 164, 1 to 50, 50 to 100, 52 to 142, 100 to 150, or 150 to 188 of SEQ ID NO:12. In certain embodiments, the CD3zeta polypeptide comprises or has an amino acid sequence of amino acids 52 to 142 of SEQ ID NO:12.

SEQ ID NO: 12:
MKWKVSVLACILHVRFPGAEAQSFGLLDPKLCYLLDGILFIYGVIITALY
LRAKFSRSAETAANLQDPNQLYNELNLGRREEYDVLEKKRARDPEMGGKQ
RRRNPQEGVYNALQKDKMAEAYSEIGTKGERRRGKGHDGLYQDSHFQAVQ
FGNRREREGSELTRTLGLRARPKACRHKKPLSLPAAVS

In certain embodiments, the CD3zeta polypeptide comprises or has the amino acid sequence set forth in SEQ ID NO: 13.

SEQ ID NO: 13:
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In certain non-limiting embodiments, an intracellular signaling domain of the CAR further comprises at least a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include, for example, a CD28 polypeptide, a 4-1BB polypeptide, an OX40 polypeptide, an ICOS polypeptide, a DAP-10 polypeptide, a DAP-12 polypeptide, a 2B4 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands include, but are not limited to, CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of an NK cell. CARs comprising an intracellular signaling domain that comprises a co-stimulatory signaling region comprising 4-1BB, ICOS or DAP-10 are disclosed in U.S. Pat. No. 7,446,190; incorporated herein by reference (i.e., a nucleotide sequence encoding 4-1BB is set forth in SEQ ID NO:15 and the protein sequence is set forth in NP_001551.2; a nucleotide sequence encoding ICOS is set forth in SEQ ID NO:16 and the protein sequence is set forth in NP_036224.1, and a nucleotide sequence encoding DAP-10 is set forth in SEQ ID NO: 17 and the protein sequence is set forth in NP_055081.1), which U.S. Pat. No. 7,446,190 and Ref Protein sequences are herein incorporated by reference in their entirety.

4-1BB
SEQ ID NO: 15
ATGGGAAACAGCTGTTACAACATAGTAGCCACTCTGTTGCTGGTCCTCAA
CTTTGAGAGGACAAGATCATTGCAGGATCCTTGTAGTAACTGCCCAGCTG
GTACATTCTGTGATAATAACAGGAATCAGATTTGCAGTCCCTGTCCTCCA
AATAGTTTCTCCAGCGCAGGTGGACAAAGGACCTGTGACATATGCAGGCA
GTGTAAAGGTGTTTTCAGGACCAGGAAGGAGTGTTCCTCCACCAGCAATG
CAGAGTGTGACTGCACTCCAGGGTTTCACTGCCTGGGGGCAGGATGCAGC
ATGTGTGAACGGATTGTAAACAAGGTCAAGAACTGACAAAAAAAGGTTG
TAAAGACTGTTGCTTTGGGACATTTAACGATCAGAAACGTGGCATCTGTC
GACCCTGGACAAACTGTTCTTTGGATGGAAAGTCTGTGCTTGTGAATGGG
ACGAAGGAGAGGGACGTGGTCTGTGGACCATCTCCAGCCGACCTCTCTCC
GGGAGCATCCTCTGTGACCCCGCCTGCCCCTGCGAGAGAGCCAGGACACT
CTCCGCAGATCATCTCCTTCTTTCTTGCGCTGACGTCGACTGCGTTGCTC
TTCCTGCTGTTCTTCCTCACGCTCCGTTTCTCTGTTGTTAAACGGGGCAG
AAAGAAACTCCTGTATATATTCAAACAACCATTTATGAGACCAGTACAAA
CTACTCAAGAGGAAGATGGCTGTAGCTGCCGATTTCCAGAAGAAGAAGAA
GGAGGATGTGAACTGTGA
ICOS
SEQ ID NO: 16
ATGAAGTCAGGCCTCTGGTATTTCTTTCTCTTCTGCTTGCGCATTAAAGT
TTTAACAGGAGAAATCAATGGTTCTGCCAATTATGAGATGTTTATATTTC
ACAACGGAGGTGTACAAATTTTATGCAAATATCCTGACATTGTCCAGCAA
TTTAAAATGCAGTTGCTGAAAAGGGGGGCAAATACTCTGCGATCTCACTA
AGACAAAAGGAAGTGGAAACACAGTGTCCATTAAGAGTCTGAAATTCTGC
CATTCTCAGTTATCCAACAACAGTGTCTCTTTTTTTCTACAACCTTGGAC
CATTCTCATGCCAACTATTACTTCTGCAACCTATCAATTTTTGATCCTCC
TCCTTTTAAAGTAACTCTTACAGGAGGATATTTGCATATTTATGAAATCA
CAACTTTGTTGCCAGCTGAAGTTCTGGTTACCCATAGGATGTGCAGCCTT
TGTTGTAGTCTGCATTTTGGGATGCATACTTATTTGTTGGCTTACAAAAA
AGAAGTATTCATCCAGTGTGCACGACCCTAACGGTGAATACATGTTCATG
AGAGCAGTGAACACAGCCAAAAAATCTAGACTCACAGATGTGACCCTATA
A
DAP-10
SEQ ID NO: 17
ATGATCCATCTGGGTCACATCCTCTTCCTGCTTTTGCTCCCAGTGGCTGC
AGCTCAGACGACCCCAGGAGAGAGATCATCACTCCCTGCCTTTTACCCTG
GCACTTCAGGCTCCTGTTCCGGATGTGGGTCCCTCTCTCTGCCGCTCCTG
GCAGGCCTCGTGGCTGCTGATGCGGTGGCATCGCTGCTCATCGTGGGGGC
GGTGTTCCTGTGCGCACGCCCACGCCGCAGCCCCGCCCAAGAAGATGGCA
AAGTCTACATCAACATGCCAGGCAGGGGCTGA

In certain embodiments, the intracellular signaling domain of the CAR comprises a co-stimulatory signaling region that comprises a CD28 polypeptide. The CD28 polypeptide can have an amino acid sequence that is at least about 85%, about 90%, about 95%, about 96%, about 97%, about 98%, about 99% or 100% identical to the sequence having a NCBI Reference No: P10747 (incorporated herein by reference) or NP_006130 (SEQ ID NO:10), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO:10 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO:10. In certain embodiments, the intracellular signaling domain of the CAR comprises a co-stimulatory signaling region that comprises a CD28 polypeptide having an amino acid sequence of amino acids 180 to 220 of SEQ ID NO:10.

In certain embodiments, the CD28 polypeptide has an amino acid sequence that is at least about 85%, about 90%, about 95%, about 96%, about 97%, about 98%, about 99% or about 100% identical to the sequence having a NCBI Reference No: NP_031668.3 (SEQ ID NO:14), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO:14 which is at least about 20, or at least about 30, or at least about 40, or at least about 50, and up to 218 amino acids in length. Alternatively or additionally, in non-limiting various embodiments, the CD28 polypeptide has an amino acid sequence of amino acids 1 to 218, 1 to 50, 50 to 100, 100 to 150, 114 to 220, 150 to 200, 178 to 218, or 200 to 220 of SEQ ID NO:14. In certain embodiments, the co-stimulatory signaling region of a presently disclosed CAR comprises a CD28 polypeptide that comprises or has the amino acids 178 to 218 of SEQ ID NO:14.

SEQ ID NO: 14:
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the intracellular signaling domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or about 100% identical to the sequence set forth in NCBI Reference No: P41273 (incorporated herein by reference) or NP_001552.2 (SEQ ID NO:18) or fragments thereof, and/or may optionally comprise up to one or up to two or up to three conservative amino acid substitutions.

SEQ ID NO: 18:
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

An OX40 polypeptide can have an amino acid sequence that is at least about 85%, about 90%, about 95%, about 96%, about 97%, about 98%, about 99% or about 100% identical to the sequence having a NCBI Reference No: P43489 (incorporated herein by reference) or NP_003318.1 (SEQ ID NO:19), or fragments thereof, and/or may optionally comprise up to one or up to two or up to three conservative amino acid substitutions.

SEQ ID NO: 19:
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

An ICOS polypeptide can have an amino acid sequence that is at least about 85%, about 90%, about 95%, about 96%, about 97%, about 98%, about 99% or about 100% identical to the sequence having a NCBI Reference No: NP_036224.1 (SEQ ID NO:20) or fragments thereof, and/or may optionally comprise up to one or up to two or up to three conservative amino acid substitutions.

SEQ ID NO: 20:
MKSGLWYFFLFCLRIKVLTGEINGSANYEMFIFHNGGVQILCKYPDIVQ
QFKMQLLKGGQILCDLIKTKGSGNTVSIKSLKFCHSQLSNNSVSFFLYN
LDHSHANYYFCNLSIFDPPPFKVTLIGGYLHIYESQLCCQLKFWLPIGC
AAFVVVCILGCILICWLTKKKYSSSVHDPNGEYMFMRAVNTAKKSRLTD
VTL

A DAP-12 polypeptide can have an amino acid sequence of a co-stimulatory region that is at least about 85%, about 90%, about 95%, about 96%, about 97%, about 98%, about 99% or about 100% identical to the sequence having a NCBI Reference No: NP_003323.1, NP_001166986.1, NP_001166985.1 or NP_937758.1 (each incorporated herein by reference) or fragments thereof, and/or may optionally comprise up to one or up to two or up to three conservative amino acid substitutions.

A 2B4 polypeptide can have an amino acid sequence of a co-stimulatory region that is at least about 85%, about 90%, about 95%, about 96%, about 97%, about 98%, about 99% or about 100% identical to the sequence having a NCBI Reference No: NP_057466.1, NP_001160135.1 or NP_001160136.1 (each incorporated herein by reference) or fragments thereof, and/or may optionally comprise up to one or up to two or up to three conservative amino acid substitutions. Additional Genes

In some embodiments, a CAR construct further includes a gene(s) encoding an additional gene product, such as a cytokine or a transfection marker. In some embodiments, the additional gene encodes a cytokine, such as human IL-15 (U.S. Pat. No. 9,931,377; SEQ ID NO:68), IL-18 or IL-12. In some embodiments, the additional gene encodes a transfection marker or suicide gene such as truncated EGFR (tEGFR) (see WO2011/056894; SEQ ID NO:67) or iCaps9 (WO2013/040371), the sequences of which are incorporated by reference herein.

tEGFR
SEQ ID NO: 67
RKVCNGIGIGEFKDSLSINATNIKHFKNCTSISGDLHILPVAFRGDSFT
HTPPLDPQELDILKTVKEITGFLLIQAWPENRTDLHAFENLEIIRGRTK
QHGQFSLAVVSLNITSLGLRSLKEISDGDVIISGNKNLCYANTINWKKL
FGTSGQKTKIISNRGENSCKATGQVCHALCSPEGCWGPEPRDCVSCRNV
SRGRECVDKCNLLEGEPREFVENSECIQCHPECLPQAMNITCTGRGPDN
CIQCAHYIDGPHCVKTCPAGVMGENNTLVWKYADAGHVCHLCHPNCTYG
CTGPGLEGCPTNGPKIPSIATGMVGALLLLLVVALGIGLFM
IL-15
SEQ ID NO: 68
RISKPHLRSISIQCYLCLLLNSHFLTEAGIHVFILGCFSAGLPKTEANW
VNVISDLKKIEDLIQSMHIDATLYTESDVHPSCKVTAMKCFLLELQVIS
LESGDASIHDTVENLIILANNSLSSNGNVTESGCKECEELEEKNIKEFL
QSFVHIVQMFINTS

### Exemplary CAR Constructs

In certain embodiments, a CAR comprises an extracellular antigen-binding domain that binds to CD19, a transmembrane domain comprising a CD28 polypeptide, and an intracellular signaling domain comprising a CD3zeta polypeptide and a co-stimulatory signaling region comprising a CD28 polypeptide. In certain embodiments, the CAR is designated 1928z. In certain embodiments, 1928z is a protein having at least about 85%, about 90%, about 95%, about 96%, about 97%, about 98%, about 99% or about 100% identical to the amino acid sequence set forth in SEQ ID NO:21. The protein sequence includes a CD8 leader sequence at amino acids 1-18, and is able to bind human CD19.

SEQ ID NO: 21
MALPVTALLLPLALLLHAEVKLQQSGAELVRPGSSVKISCKASGYAFSS
YWMNWVKQRPGQGLEWIGQIYPGDGDTNYNGKFKGQATLTADKSSSTAY
MQLSGLTSEDSAVYFCARKTISSVVDFYPDYWGQGTTVTVSSGGGGSGG
GGSGGGGSDIELTQSPKFMSTSVGDRVSVTCKASQNVGTNVAWYQQKPG
QSPKPLIYSATYRNSGVPDRFTGSGSGTDFTLTITNVQSKDLADYFCQQ
YNRYPYTSGGGTKLEIKRAAAIEVMYPPPYLDNEKSNGTIIHVKGKHLC
PSPLFPGPSKPFWVLVVVGGVLACYSLLVTVAFIIFWVRSKRSRLLHSD
YMNMTPRRPGPTRKHYQPYAPPRDFAAYRSRVKFSRSAEPPAYQQGQNQ
LYNELNLGRREEYDVLDKRRGRDPEMGGKPRRKNPQEGLYNELQKDKMA
EAYSEIGMKGERRRGKGHDGLYQGLSTATKDTYDALHMQALPPR

In another embodiment, a CAR having an extracellular antigen-binding domain that binds to CD19, a hinge spacer from human IgG₄, a transmembrane domain comprising a CD28 transmembrane domain, a 4-1BB co-stimulatory signaling region, and a CD3ζ intracellular signaling domain is provided. In certain embodiments, the CAR is designated 1928z1. In certain embodiments, 1928z1 is a protein having at least about 85%, about 90%, about 95%, about 96%, about 97%, about 98%, about 99% or about 100% identical to the amino acid sequence set forth in SEQ ID NO:22.

SEQ ID NO: 22
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPMFWVLVVVGGVLACY
SLLVTVAFIIFWVKRGRKKLLYIFKQPFMRPVQTTQEEDGCSCRFPEEE
EGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPRL

In certain embodiments, a CAR comprises an extracellular antigen-binding domain that binds to MUC16, a transmembrane domain comprising a CD28 polypeptide, and an intracellular signaling domain comprising a CD3zeta polypeptide and a co-stimulatory signaling region comprising a CD28 polypeptide. In certain embodiments, the CAR is designated 4H1128z. In certain embodiments, 4H1128z is a protein having at least about 85%, about 90%, about 95%, about 96%, about 97%, about 98%, about 99% or about 100% identical to the amino acid sequence set forth in SEQ ID NO:23. The protein includes a CD8 leader sequence at amino acids 1-18 and binds to the MUC-16 ectodomain.

SEQ ID NO: 23
MALPVTALLLPLALLLHAEVKLQESGGGFVKPGGSLKVSCAASGFTFSS
YAMSWVRLSPEMRLEWVATISSAGGYIFYSDSVQGRFTISRDNAKNTLH
LQMGSLRSGDTAMYYCARQGFGNYGDYYAMDYWGQGTTVTVSSGGGGSG
GGGSGGGGSDIELTQSPSSLAVSAGEKVTMSCKSSQSLLNSRTRKNQLA
WYQQKPGQSPELLIYWASTRQSGVPDRFTGSGSGTDFTLTISSVQAEDL
AVYYCQQSYNLLTFGPGTKLEIKRAAAIEVMYPPPYLDNEKSNGTIIHV
KGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSAEPPAY
QQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPP
R

In certain embodiments, a CAR comprises an extracellular antigen-binding domain that binds to CD19, a transmembrane domain comprising a CD8 polypeptide, and an intracellular signaling domain comprising a CD3zeta polypeptide and a co-stimulatory signaling region comprising a 4-1BB polypeptide. In certain embodiments, the CAR is 19BBz. An exemplary protein sequence of the 19BBz polypeptide is set forth in SEQ ID NO:24.

SEQ ID NO: 24:
MALPVTALLLPLALLLHAEVKLQQSGAELVRPGSSVKISCKASGYAFSS
YWMNWVKQRPGQGLEWIGQIYPGDGDTNYNGKFKGQATLTADKSSSTAY
MQLSGLTSEDSAVYFCARKTISSVVDFYFDYWGQGTTVTVSSGGGGSGG
GGSGGGGSDIELTQSPKFMSTSVGDRVSVTCKASQNVGTNVAWYQQKPG
QSPKPLIYSATYRNSGVPDRFTGSGSGTDFTLTITNVQSKDLADYFCQQ
YNRYPYTSGGGTKLEIKRAAAPTTTPAPRPPTPAPTIASQPLSLRPEAC
RPAAGGAVHTRGLDFACDIYIWAPLAGTCGVLLLSLVITLYCNKRGRKK
LLYIFKQPFMRPVQTTQEEDGCSCRFPEEEEGGCELRVKFSRSAEPPAY
QQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPP
R

In certain embodiments, a CAR having an extracellular antigen-binding domain that binds to CD19, a hinge spacer from human IgG₄, a transmembrane domain comprising a CD28 transmembrane domain, a 4-1BB co-stimulatory signaling region, and a CD3zeta intracellular signaling domain is provided. An exemplary CD19 CAR protein sequence, that also includes a truncated EGFR (tEGFR) polypeptide attached to the carboxy terminal portion of the CAR by a self-cleaving 2A peptide, is set forth in SEQ ID NO:61.

SEQ ID NO: 61:
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPMFWVLVVVGGVLACY
SLLVTVAFIIFWVKRGRKKLLYIFKQPFMRPVQTTQEEDGCSCRFPEEE
EGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPRLEGGGEGRGSLLTCGDVEENPGPRMLLLV
TSLLLCELPHPAFLLIPRKVCNGIGIGEFKDSLSINATNIKHFKNCTSI
SGDLHILPVAIRGDSFTHTPPLDPQELDILKTVKEITGFLLIQAWPENR
TDLHAFENLEIIRGRTKQHGQFSLAVVSLNITSLGLRSLKEISDGDVII
SGNKNLCYANTINWKKLFGTSGQKTKIISNRGENSCKATGQVCHALCSP
EGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKY
ADAGHVCHLCHPNCTYGCTGPGLEGCPTNGPKIPSIATGMVGALLLLLV
VALGIGLFM

In certain embodiments, a CAR having an extracellular antigen-binding domain that binds to CD19, a hinge spacer from human IgG₄, a transmembrane domain of a CD28 transmembrane domain, a 4-1BB co-stimulatory signaling region, and a CD3ζ intracellular signaling domain is provided. An exemplary CD19 CAR protein sequence, that also includes a human IL-15 attached to the carboxy terminal portion of the CAR by a self-cleaving 2A peptide, is set forth in SEQ ID NO:62.

SEQ ID NO: 62:
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPMFWVLVVVGGVLACY
SLLVTVAFIIFWVKRGRKKLLYIFKQPFMRPVQTTQEEDGCSCRFPEEE
EGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPRLEGGGEGRGSLLTCGDVEENPGPRMRISK
PHLRSISIQCYLCLLLNSHFLTEAGIHVFILGCFSAGLPKTEANWVNVI
SDLKKIEDLIQSMHIDATLYTESDVHPSCKVTAMKCFLLELQVISLESG
DASIHDTVENLIILANNSLSSNGNVTESGCKECEELEEKNIKEFLQSFV
HIVQMFINTS

The corresponding nucleic acid sequence that can encode the construct in SEQ ID NO:62 is set forth in SEQ ID NO:69.

SEQ ID NO: 69:
ATGCTGCTGCTGGTGACCAGCCTGCTGCTGTGCGAGCTGCCCCACCCCG
CCTTTCTGCTGATCCCCGACATCCAGATGACCCAGACCACCTCCAGCCT
GAGCGCCAGCCTGGGCGACCGGGTGACCATCAGCTGCCGGGCCAGCCAG
GACATCAGCAAGTACCTGAACTGGTATCAGCAGAAGCCCGACGGCACCG
TCAAGCTGCTGATCTACCACACCAGCCGGCTGCACAGCGGCGTGCCCAG
CCGGTTTAGCGGCAGCGGCTCCGGCACCGACTACAGCCTGACCATCTCC
AACCTGGAACAGGAAGATATCGCCACCTACTTTTGCCAGCAGGGCAACA
CACTGCCCTACACCTTTGGCGGCGGAACAAAGCTGGAAATCACCGGCAG
CACCTCCGGCAGCGGCAAGCCTGGCAGCGGCGAGGGCAGCACCAAGGGC
GAGGTGAAGCTGCAGGAAAGCGGCCCTGGCCTGGTGGCCCCCAGCCAGA
GCCTGAGCGTGACCTGCACCGTGAGCGGCGTGAGCCTGCCCGACTACGG
CGTGAGCTGGATCCGGCAGCCCCCCAGGAAGGGCCTGGAATGGCTGGGC
GTGATCTGGGGCAGCGAGACCACCTACTACAACAGCGCCCTGAAGAGCC
GGCTGACCATCATCAAGGACAACAGCAAGAGCCAGGTGTTCCTGAAGAT
GAACAGCCTGCAGACCGACGACACCGCCATCTACTACTGCGCCAAGCAC
TACTACTACGGCGGCAGCTACGCCATGGACTACTGGGGCCAGGGCACCA
GCGTGACCGTGAGCAGCGAATCTAAGTACGGACCGCCCTGCCCCCCTTG
CCCTATGTTCTGGGTGCTGGTGGTGGTCGGAGGCGTGCTGGCCTGCTAC
AGCCTGCTGGTCACCGTGGCCTTCATCATCTTTTGGGTGAAACGGGGCA
GAAAGAAACTCCTGTATATATTCAAACAACCATTTATGAGACCAGTACA
AACTACTCAAGAGGAAGATGGCTGTAGCTGCCGATTTCCAGAAGAAGAA
GAAGGAGGATGTGAACTGAGGGTGAAGTTCAGCAGAAGCGCCGACGCCC
CTGCCTACCAGCAGGGCCAGAATCAGCTGTACAACGAGCTGAACCTGGG
CAGAAGGGAAGAGTACGACGTCCTGGATAAGCGGAGAGGCCGGGACCCT
GAGATGGGCGGCAAGCCTCGGCGGAAGAACCCCCAGGAAGGCCTGTATA
ACGAACTGCAGAAAGACAAGATGGCCGAGGCCTACAGCGAGATCGGCAT
GAAGGGCGAGCGGAGGCGGGGCAAGGGCCACGACGGCCTGTATCAGGGC
CTGTCCACCGCCACCAAGGATACCTACGACGCCCTGCACATGCAGGCCC
TGCCCCCAAGGCTCGAGGGCGGCGGAGAGGGCAGAGGAAGTCTTCTAAC
ATGCGGTGACGTGGAGGAGAATCCCGGCCCTAGGATGCGGATTTCCAAA
CCTCACCTGCGCTCTATCTCTATCCAGTGCTATCTGTGCCTGCTGCTGA
ACTCACATTTCCTGACCGAAGCCGGCATCCACGTGTTCATCCTGGGCTG
CTTTTCCGCCGGCCTGCCAAAGACCGAGGCAAACTGGGTGAATGTGATC
TCTGACCTGAAGAAGATCGAGGATCTGATCCAGAGCATGCACATCGACG
CCACCCTGTACACAGAGTCCGATGTGCACCCTTCTTGCAAGGTGACAGC
CATGAAGTGTTTCCTGCTGGAGCTGCAGGTCATCAGCCTGGAGAGCGGC
GACGCCTCTATCCACGATACCGTGGAGAACCTGATCATCCTGGCCAACA
ATAGCCTGAGCAGCAACGGCAATGTGACAGAGTCCGGCTGCAAGGAGTG
TGAGGAGCTGGAGGAGAAGAATATCAAAGAGTTCCTGCAGTCATTCGTC
CATATCGTCCAGATGTTTATCAATACCTCCTAA

In certain embodiments, a CAR having an extracellular antigen-binding domain that binds to CD19, a hinge spacer from human IgG₄, a transmembrane domain of a CD28 transmembrane domain, a 2B4 co-stimulatory signaling region, and a CD3zeta intracellular signaling domain is provided. An exemplary CD19 CAR protein sequence, that also includes a human IL-15 attached to the carboxy terminal portion of the CAR by a self-cleaving 2A peptide, is set forth in SEQ ID NO:70.

SEQ ID NO: 70:
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPMFWVLVVVGGVLACY
SLLVTVAFIIFWVWRRKRKEKQSETSPKEFLTIYEDVKDLKTRRNHEQE
QTFPGGGSTIYSMIQSQSSAPTSQEPAYTLYSLIQPSRKSGSRKRNHSP
SFNSTIYEVIGKSQPKAQNPARLSRKELENFDVYSRVKFSRSADAPAYQ
QGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
LEGGGEGRGSLLTCGDVEENPGPRMRISKPHLRSISIQCYLCLLLNSHF
LTEAGIHVFILGCFSAGLPKTEANWVNVISDLKKIEDLIQSMHIDATLY
TESDVHPSCKVTAMKCFLLELQVISLESGDASIHDTVENLIILANNSLS
SNGNVTESGCKECEELEEKNIKEFLQSFVHIVQMFINTS

The corresponding nucleic acid sequence encoding the SEQ ID NO: 70 construct is set forth in SEQ ID NO:71.

SEQ ID NO: 71:
ATGCTGCTGCTGGTGACCAGCCTGCTGCTGTGCGAGCTGCCCCACCCCG
CCTTTCTGCTGATCCCCGACATCCAGATGACCCAGACCACCTCCAGCCT
GAGCGCCAGCCTGGGCGACCGGGTGACCATCAGCTGCCGGGCCAGCCAG
GACATCAGCAAGTACCTGAACTGGTATCAGCAGAAGCCCGACGGCACCG
TCAAGCTGCTGATCTACCACACCAGCCGGCTGCACAGCGGCGTGCCCAG
CCGGTTTAGCGGCAGCGGCTCCGGCACCGACTACAGCCTGACCATCTCC
AACCTGGAACAGGAAGATATCGCCACCTACTTTTGCCAGCAGGGCAACA
CACTGCCCTACACCTTTGGCGGCGGAACAAAGCTGGAAATCACCGGCAG
CACCTCCGGCAGCGGCAAGCCTGGCAGCGGCGAGGGCAGCACCAAGGGC
GAGGTGAAGCTGCAGGAAAGCGGCCCTGGCCTGGTGGCCCCCAGCCAGA
GCCTGAGCGTGACCTGCACCGTGAGCGGCGTGAGCCTGCCCGACTACGG
CGTGAGCTGGATCCGGCAGCCCCCCAGGAAGGGCCTGGAATGGCTGGGC
GTGATCTGGGGCAGCGAGACCACCTACTACAACAGCGCCCTGAAGAGCC
GGCTGACCATCATCAAGGACAACAGCAAGAGCCAGGTGTTCCTGAAGAT
GAACAGCCTGCAGACCGACGACACCGCCATCTACTACTGCGCCAAGCAC
TACTACTACGGCGGCAGCTACGCCATGGACTACTGGGGCCAGGGCACCA
GCGTGACCGTGAGCAGCGAATCTAAGTACGGACCGCCCTGCCCCCCTTG
CCCTATGTTCTGGGTGCTGGTGGTGGTCGGAGGCGTGCTGGCCTGCTAC
AGCCTGCTGGTCACCGTGGCCTTCATCATCTTTTGGGTGTGGAGGAGGA
AGAGGAAGGAGAAGCAGAGCGAGACAAGCCCTAAGGAGTTTCTGACAAT
CTATGAAGACGTGAAGGACCTGAAGACACGGAGAAACCACGAGCAGGAG
CAGACCTTCCCTGGAGGAGGCAGCACAATCTACTCCATGATCCAGTCTC
AGAGCAGCGCCCCCACCTCCCAGGAGCCTGCCTACACACTGTATAGCCT
GATCCAGCCATCCCGGAAGTCTGGCAGCAGGAAGCGCAACCACTCCCCC
TCTTTTAATTCTACCATCTATGAAGTGATCGGCAAGAGCCAGCCCAAGG
CACAGAACCCCGCACGACTGAGCAGGAAGGAACTGGAGAACTTTGATGT
CTACTCTAGGGTGAAGTTCAGCAGAAGCGCCGACGCCCCTGCCTACCAG
CAGGGCCAGAATCAGCTGTACAACGAGCTGAACCTGGGCAGAAGGGAAG
AGTACGACGTCCTGGATAAGCGGAGAGGCCGGGACCCTGAGATGGGCGG
CAAGCCTCGGCGGAAGAACCCCCAGGAAGGCCTGTATAACGAACTGCAG
AAAGACAAGATGGCCGAGGCCTACAGCGAGATCGGCATGAAGGGCGAGC
GGAGGCGGGGCAAGGGCCACGACGGCCTGTATCAGGGCCTGTCCACCGC
CACCAAGGATACCTACGACGCCCTGCACATGCAGGCCCTGCCCCCAAGG
CTCGAGGGCGGCGGAGAGGGCAGAGGAAGTCTTCTAACATGCGGTGACG
TGGAGGAGAATCCCGGCCCTAGGATGCGGATTTCCAAACCTCACCTGCG
CTCTATCTCTATCCAGTGCTATCTGTGCCTGCTGCTGAACTCACATTTC
CTGACCGAAGCCGGCATCCACGTGTTCATCCTGGGCTGCTTTTCCGCCG
GCCTGCCAAAGACCGAGGCAAACTGGGTGAATGTGATCTCTGACCTGAA
GAAGATCGAGGATCTGATCCAGAGCATGCACATCGACGCCACCCTGTAC
ACAGAGTCCGATGTGCACCCTTCTTGCAAGGTGACAGCCATGAAGTGTT
TCCTGCTGGAGCTGCAGGTCATCAGCCTGGAGAGCGGCGACGCCTCTAT
CCACGATACCGTGGAGAACCTGATCATCCTGGCCAACAATAGCCTGAGC
AGCAACGGCAATGTGACAGAGTCCGGCTGCAAGGAGTGTGAGGAGCTGG
AGGAGAAGAATATCAAAGAGTTCCTGCAGTCATTCGTCCATATCGTCCA
GATGTTTATCAATACCTCCTAA

In certain embodiments, a CAR having an extracellular antigen-binding domain that binds to CD19, a hinge spacer from human IgG₄, a transmembrane domain of a NKG2D transmembrane domain, a 4-1BB co-stimulatory signaling region, and a CD3zeta intracellular signaling domain is provided. Use of the NKG2Ds transmembrane domain has been used previously (Xu et al., J. Hematol. Oncol. 12:49, 2019) although some suggest that because NKGD2 is a type II membrane protein it should be inserted in the opposite orientation. An exemplary CD19 CAR protein sequence, that also includes a human IL-15 attached to the carboxy terminal portion of the CAR by a self-cleaving 2A peptide, is set forth in SEQ ID NO:72.

SEQ ID NO: 72:
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPPFFFCCFIAVAMGIR
FIIMVTKRGRKKLLYIFKQPFMRPVQTTQEEDGCSCRFPEEEEGGCELR
VKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKD
TYDALHMQALPPRLEGGGEGRGSLLTCGDVEENPGPRMRISKPHLRSIS
IQCYLCLLLNSHFLTEAGIHVFILGCFSAGLPKTEANWVNVISDLKKIE
DLIQSMHIDATLYTESDVHPSCKVTAMKCFLLELQVISLESGDASIHDT
VENLIILANNSLSSNGNVTESGCKECEELEEKNIKEFLQSFVHIVQMFI
NTS

The corresponding nucleic acid sequence encoding the SEQ ID NO: 72 construct is set forth in SEQ ID NO:73.

SEQ ID NO: 73:
ATGCTGCTGCTGGTGACCAGCCTGCTGCTGTGCGAGCTGCCCCACCCCG
CCTTTCTGCTGATCCCCGACATCCAGATGACCCAGACCACCTCCAGCCT
GAGCGCCAGCCTGGGCGACCGGGTGACCATCAGCTGCCGGGCCAGCCAG
GACATCAGCAAGTACCTGAACTGGTATCAGCAGAAGCCCGACGGCACCG
TCAAGCTGCTGATCTACCACACCAGCCGGCTGCACAGCGGCGTGCCCAG
CCGGTTTAGCGGCAGCGGCTCCGGCACCGACTACAGCCTGACCATCTCC
AACCTGGAACAGGAAGATATCGCCACCTACTTTTGCCAGCAGGGCAACA
CACTGCCCTACACCTTTGGCGGCGGAACAAAGCTGGAAATCACCGGCAG
CACCTCCGGCAGCGGCAAGCCTGGCAGCGGCGAGGGCAGCACCAAGGGC
GAGGTGAAGCTGCAGGAAAGCGGCCCTGGCCTGGTGGCCCCCAGCCAGA
GCCTGAGCGTGACCTGCACCGTGAGCGGCGTGAGCCTGCCCGACTACGG
CGTGAGCTGGATCCGGCAGCCCCCCAGGAAGGGCCTGGAATGGCTGGGC
GTGATCTGGGGCAGCGAGACCACCTACTACAACAGCGCCCTGAAGAGCC
GGCTGACCATCATCAAGGACAACAGCAAGAGCCAGGTGTTCCTGAAGAT
GAACAGCCTGCAGACCGACGACACCGCCATCTACTACTGCGCCAAGCAC
TACTACTACGGCGGCAGCTACGCCATGGACTACTGGGGCCAGGGCACCA
GCGTGACCGTGAGCAGCGAATCTAAGTACGGACCGCCCTGCCCCCCTTG
CCCTCCCTTCTTTTTCTGCTGTTTTATCGCCGTGGCTATGGGCATCCGG
TTCATCATCATGGTGACCAAACGGGGCAGAAAGAAACTCCTGTATATAT
TCAAACAACCATTTATGAGACCAGTACAAACTACTCAAGAGGAAGATGG
CTGTAGCTGCCGATTTCCAGAAGAAGAAGAAGGAGGATGTGAACTGAGG
GTGAAGTTCAGCAGAAGCGCCGACGCCCCTGCCTACCAGCAGGGCCAGA
ATCAGCTGTACAACGAGCTGAACCTGGGCAGAAGGGAAGAGTACGACGT
CCTGGATAAGCGGAGAGGCCGGGACCCTGAGATGGGCGGCAAGCCTCGG
CGGAAGAACCCCCAGGAAGGCCTGTATAACGAACTGCAGAAAGACAAGA
TGGCCGAGGCCTACAGCGAGATCGGCATGAAGGGCGAGCGGAGGCGGGG
CAAGGGCCACGACGGCCTGTATCAGGGCCTGTCCACCGCCACCAAGGAT
ACCTACGACGCCCTGCACATGCAGGCCCTGCCCCCAAGGCTCGAGGGCG
GCGGAGAGGGCAGAGGAAGTCTTCTAACATGCGGTGACGTGGAGGAGAA
TCCCGGCCCTAGGATGCGGATTTCCAAACCTCACCTGCGCTCTATCTCT
ATCCAGTGCTATCTGTGCCTGCTGCTGAACTCACATTTCCTGACCGAAG
CCGGCATCCACGTGTTCATCCTGGGCTGCTTTTCCGCCGGCCTGCCAAA
GACCGAGGCAAACTGGGTGAATGTGATCTCTGACCTGAAGAAGATCGAG
GATCTGATCCAGAGCATGCACATCGACGCCACCCTGTACACAGAGTCCG
ATGTGCACCCTTCTTGCAAGGTGACAGCCATGAAGTGTTTCCTGCTGGA
GCTGCAGGTCATCAGCCTGGAGAGCGGCGACGCCTCTATCCACGATACC
GTGGAGAACCTGATCATCCTGGCCAACAATAGCCTGAGCAGCAACGGCA
ATGTGACAGAGTCCGGCTGCAAGGAGTGTGAGGAGCTGGAGGAGAAGAA
TATCAAAGAGTTCCTGCAGTCATTCGTCCATATCGTCCAGATGTTTATC
AATACCTCCTAA

In certain embodiments, a CAR having an extracellular antigen-binding domain that binds to CD19, a hinge spacer from human IgG₄, a transmembrane domain of a NKG2D transmembrane domain, a 2B4 co-stimulatory signaling region, and a CD33 intracellular signaling domain is provided. An exemplary CD19 CAR protein sequence, that also includes a human IL-15 attached to the carboxy terminal portion of the CAR by a self-cleaving 2A peptide, is set forth in SEQ ID NO:74.

SEQ ID NO: 74:
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSESKYGPPCPPCPPFFFCCFIAVAMGIR
FIIMVTWRRKRKEKQSETSPKEFLTIYEDVKDLKTRRNHEQEQTFPGGG
STIYSMIQSQSSAPTSQEPAYTLYSLIQPSRKSGSRKRNHSPSFNSTIY
EVIGKSQPKAQNPARLSRKELENFDVYSRVKFSRSADAPAYQQGQNQLY
NELNLGRREEYDVLDKRRGRDPEMGGKPRRKNPQEGLYNELQKDKMAEA
YSEIGMKGERRRGKGHDGLYQGLSTATKDTYDALHMQALPPRLEGGGEG
RGSLLTCGDVEENPGPRMRISKPHLRSISIQCYLCLLLNSHFLTEAGIH
VFILGCFSAGLPKTEANWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTS

The corresponding nucleic acid sequence encoding the SEQ ID NO: 74 construct is set forth in SEQ ID NO:75.

SEQ ID NO: 75:
ATGCTGCTGCTGGTGACCAGCCTGCTGCTGTGCGAGCTGCCCCACCCCG
CCTTTCTGCTGATCCCCGACATCCAGATGACCCAGACCACCTCCAGCCT
GAGCGCCAGCCTGGGCGACCGGGTGACCATCAGCTGCCGGGCCAGCCAG
GACATCAGCAAGTACCTGAACTGGTATCAGCAGAAGCCCGACGGCACCG
TCAAGCTGCTGATCTACCACACCAGCCGGCTGCACAGCGGCGTGCCCAG
CCGGTTTAGCGGCAGCGGCTCCGGCACCGACTACAGCCTGACCATCTCC
AACCTGGAACAGGAAGATATCGCCACCTACTTTTGCCAGCAGGGCAACA
CACTGCCCTACACCTTTGGCGGCGGAACAAAGCTGGAAATCACCGGCAG
CACCTCCGGCAGCGGCAAGCCTGGCAGCGGCGAGGGCAGCACCAAGGGC
GAGGTGAAGCTGCAGGAAAGCGGCCCTGGCCTGGTGGCCCCCAGCCAGA
GCCTGAGCGTGACCTGCACCGTGAGCGGCGTGAGCCTGCCCGACTACGG
CGTGAGCTGGATCCGGCAGCCCCCCAGGAAGGGCCTGGAATGGCTGGGC
GTGATCTGGGGCAGCGAGACCACCTACTACAACAGCGCCCTGAAGAGCC
GGCTGACCATCATCAAGGACAACAGCAAGAGCCAGGTGTTCCTGAAGAT
GAACAGCCTGCAGACCGACGACACCGCCATCTACTACTGCGCCAAGCAC
TACTACTACGGCGGCAGCTACGCCATGGACTACTGGGGCCAGGGCACCA
GCGTGACCGTGAGCAGCGAATCTAAGTACGGACCGCCCTGCCCCCCTTG
CCCTCCCTTCTTTTTCTGCTGTTTTATCGCCGTGGCTATGGGCATCCGG
TTCATCATCATGGTGACCTGGAGGAGGAAGAGGAAGGAGAAGCAGAGCG
AGACAAGCCCTAAGGAGTTTCTGACAATCTATGAAGACGTGAAGGACCT
GAAGACACGGAGAAACCACGAGCAGGAGCAGACCTTCCCTGGAGGAGGC
AGCACAATCTACTCCATGATCCAGTCTCAGAGCAGCGCCCCCACCTCCC
AGGAGCCTGCCTACACACTGTATAGCCTGATCCAGCCATCCCGGAAGTC
TGGCAGCAGGAAGCGCAACCACTCCCCCTCTTTTAATTCTACCATCTAT
GAAGTGATCGGCAAGAGCCAGCCCAAGGCACAGAACCCCGCACGACTGA
GCAGGAAGGAACTGGAGAACTTTGATGTCTACTCTAGGGTGAAGTTCAG
CAGAAGCGCCGACGCCCCTGCCTACCAGCAGGGCCAGAATCAGCTGTAC
AACGAGCTGAACCTGGGCAGAAGGGAAGAGTACGACGTCCTGGATAAGC
GGAGAGGCCGGGACCCTGAGATGGGCGGCAAGCCTCGGCGGAAGAACCC
CCAGGAAGGCCTGTATAACGAACTGCAGAAAGACAAGATGGCCGAGGCC
TACAGCGAGATCGGCATGAAGGGCGAGCGGAGGCGGGGCAAGGGCCACG
ACGGCCTGTATCAGGGCCTGTCCACCGCCACCAAGGATACCTACGACGC
CCTGCACATGCAGGCCCTGCCCCCAAGGCTCGAGGGCGGCGGAGAGGGC
AGAGGAAGTCTTCTAACATGCGGTGACGTGGAGGAGAATCCCGGCCCTA
GGATGCGGATTTCCAAACCTCACCTGCGCTCTATCTCTATCCAGTGCTA
TCTGTGCCTGCTGCTGAACTCACATTTCCTGACCGAAGCCGGCATCCAC
GTGTTCATCCTGGGCTGCTTTTCCGCCGGCCTGCCAAAGACCGAGGCAA
ACTGGGTGAATGTGATCTCTGACCTGAAGAAGATCGAGGATCTGATCCA
GAGCATGCACATCGACGCCACCCTGTACACAGAGTCCGATGTGCACCCT
TCTTGCAAGGTGACAGCCATGAAGTGTTTCCTGCTGGAGCTGCAGGTCA
TCAGCCTGGAGAGCGGCGACGCCTCTATCCACGATACCGTGGAGAACCT
GATCATCCTGGCCAACAATAGCCTGAGCAGCAACGGCAATGTGACAGAG
TCCGGCTGCAAGGAGTGTGAGGAGCTGGAGGAGAAGAATATCAAAGAGT
TCCTGCAGTCATTCGTCCATATCGTCCAGATGTTTATCAATACCTCCTA
A

### Expression Constructs

In certain embodiments, a CAR can further be expressed from a nucleic acid comprising an inducible promoter, for expressing nucleic acid sequences in human cells. Promoters for use in expressing CAR genes can be a constitutive promoter, such as promoters for ubiquitin C (UbiC), PGK, EF-1alpha, MND (a synthetic viral promoter that contains the U3 region of a modified Moloney murine leukemia virus long terminal repeat with myeloproliferative sarcoma virus enhancer), or Chicken beta actin.

Methods of preparing genetically modified NK cell compositions and/or preparations for immunotherapy comprise introducing into the CD56⁺ (mainly NK) cells a polynucleotide(s) encoding an antigen recognizing receptor(s), such as a CAR, into the cells. The polynucleotide(s) encoding the desired molecule can be introduced into the HSPCs, before, during, or after expansion, or into HSPCs or NK cell compositions and/or preparations before, during, or after differentiation. Some embodiments relate to a method of engineering an NK cell composition and/or preparation by transforming, transducing, or transfecting an HSPC or NK cell composition and/or preparation with at least one polynucleotide encoding a CAR, TCR, or other antigen recognizing receptor, and expressing the polynucleotide in the cell. Desired polynucleotides encoding genes can be introduced into HSPCs or other cells by any method known in the art, including transfection, electroporation, microinjection, lipofection, calcium phosphate mediated transfection, infection with a viral or bacteriophage vector containing the gene sequences, cell fusion, chromosome-mediated gene transfer, microcell-mediated gene transfer, spheroplast fusion, using CRISPR or other rare-cutting endonuclease (e.g., TALE-nuclease or Cas9 endonuclease), and the like. Numerous techniques are known in the art for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985) and can be used, provided that the necessary physiological functions of the recipient cells are not disrupted. The technique should provide for the stable transfer of the gene to the cell, so that the gene is expressible by the cell and preferably heritable and expressible by its cell progeny. In some embodiments, the method of transfer includes the transfer of a selectable marker or tag sequence to the cells. The cells are then placed under selection to isolate those cells that have taken up and express the transferred gene. In a preferred embodiment, the polynucleotide(s) or genes are included in lentiviral vectors in view of being stably expressed in the cells.

In an embodiment, an antigen recognizing receptor (e.g., CAR or TCR) is introduced into HSPCs during the expansion phase and prior to differentiation, to form antigen recognizing receptor-expressing HSPCs and/or antigen recognizing receptor-expressing NK cells. In a preferred embodiment, the antigen recognizing receptor CAR is introduced into HSPCs during the expansion phase and prior to differentiation, to form CAR-HSPCs and/or CAR-NK cells. In another embodiment, an antigen recognizing receptor (e.g., CAR or TCR) is introduced into isolated enriched CD56⁺ NK cells after expansion and during the differentiation phase, to form antigen recognizing receptor-expressing HSPCs and/or antigen recognizing receptor-expressing NK cells. In a preferred embodiment, the antigen recognizing receptor CAR is introduced into isolated enriched CD56⁺ NK cells after expansion and during the differentiation phase, to form CAR-HSPCs and/or CAR-NK cells. The antigen recognizing receptor introduction, CAR introduction, cell expansion, and cell differentiation mentioned above are each carried out as described above and below.

The different methods described above involve introducing CAR, or another antigen recognizing receptor into a cell. As a non-limiting example, a CAR or TCR can be introduced as a transgene(s) encoded by one plasmid vector. The plasmid vector can also contain a selection marker which provides for identification and/or selection of cells which received said vector.

Polypeptides, such as a CAR or TCR, can be synthesized in situ in the cell as a result of the introduction of polynucleotides encoding the polypeptides into the cell.

Methods for viral mediated introduction of a polynucleotide construct into cells are known in the art, and include as non-limiting examples recombinant viral vectors (e.g., retroviruses, adenoviruses).

Cryopreservation of the NK Cell Composition and/or Preparation

An NK cell composition and/or preparation, with or without genetic engineering, can be divided and frozen in one or more bags (or units). In a preferred embodiment, from about 50 to about 500 million total cells are frozen in a single bag (or unit). In another preferred embodiment, from about 100 to about 500 million cells are frozen in a single bag (or unit). In other preferred embodiments, about 50, 100, 200, 300, 400 or 400 million cells are frozen in a single bag (or unit). In some embodiments a single bag (or unit) contains about 50 million to about 2 billion viable cells per dose. In some embodiments, a single bag (or unit) contains about 100 million, about 200 million, about 300 million, about 400 million, about 500 million, about 600 million, about 750 million, about 1 billion, about 1.5 billion or about 2 billion viable cells. In some embodiments, a single bag contains about 50 million to about 2 billion viable CD56⁺ cells per dose. In some embodiments, a single bag (or unit) contains about 100 million, about 200 million, about 300 million, about 400 million, about 500 million, about 600 million, about 750 million, about 1 billion, about 1.5 billion or about 2 billion viable CD56⁺ cells.

In a preferred embodiment, the NK cell composition and/or preparation is frozen or cryopreserved. In another embodiment, the NK cell composition and/or preparation is fresh, i.e., the cells have not been previously frozen prior to expansion or cryopreservation. The terms “frozen/freezing” and “cryopreserved/cryopreserving/cryopreservation” are used interchangeably in the present application. Cryopreservation can be by any method known in the art that preserves cells in viable form. The freezing of cells is ordinarily destructive because on cooling, water within the cell freezes, leading to injury caused by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. For a discussion, see Mazur, P., Cryobiology 14:251-272, 1977.

These injurious effects can be circumvented by (a) use of a cryoprotective agent, (b) control of the freezing rate, and (c) storage at a temperature sufficiently low to minimize degradative reactions.

Cryoprotective agents which can be used include, but are not limited to, dimethyl sulfoxide (DMSO) (Lovelock and Bishop, Nature 183:1394-1395, 1959; Ashwood-Smith, Nature 190:1204-1205, 1961); glycerol, polyvinylpyrrolidine (Rinfret, Ann. N.Y. Acad. Sci. 85:576, 1960); polyethylene glycol (Sloviter and Ravdin, Nature 196:548, 1962); albumin, dextran, sucrose, ethylene glycol, i-erythritol, D-ribitol, D-mannitol (Rowe et al., Fed. Proc. 21:157, 1962); D-sorbitol, i-inositol, D-lactose, choline chloride (Bender et al., J. Appl. Physiol. 15:520, 1960); amino acids (Phan The Tran and Bender, 1960, Exp. Cell Res. 20:651, 1960); methanol, acetamide, glycerol monoacetate (Lovelock, Biochem. J. 56:265, 1954); inorganic salts (Phan The Tran and Bender, Proc. Soc. Exp. Biol. Med. 104:388, 1960; Phan The Tran and Bender, in Radiobiology, Proceedings of the Third Australian Conference on Radiobiology, Ilbery ed., Butterworth, London, p. 59, 1961), and CryoStor® CS5 or CS10 (BioLife Solutions Inc., Bothell, WA). In a preferred embodiment, DMSO is used. For example, DMSO is used at a concentration which is nontoxic to cells. Additionally, DMSO comprises up to about 20% of the composition, up to about 15% of the composition, up to about 10% of the composition, up to about 5% of the composition, up to about 2% of the composition, up to about 1% of the composition, or up to about 0.5% of the composition. In some embodiments, addition of plasma (e.g., up to a concentration of about 20 to 25%) can augment the protective effect of DMSO. In some embodiments, addition of a human protein, such as for example, human serum albumin (e.g., up to a concentration of about 2 to 10%) can augment the protective effect of DMSO. After addition of DMSO, cells should be kept at 0° C. until freezing, since DMSO concentrations of about 1% can be toxic at temperatures above 4° C.

In another embodiment, PBS containing 20% DMSO and 8% human serum albumin (HSA), or other suitable cell freezing media is used. This mixture is then diluted 1:1 with media so that the final concentration of DMSO and HSA are 10% and 4%, respectively. The cells in this mixture are then frozen to −80° C. at a rate of 1° per minute and stored in the vapor phase of a liquid nitrogen storage tank.

A controlled slow cooling rate can be important. Different cryoprotective agents (Rapatz et al., Cryobiology 5 (1): 18-25, 1968) and different cell types have different optimal cooling rates (see e.g., Rowe and Rinfret, Blood 20:636, 1962; Rowe, Cryobiology 3 (1): 12-18, 1966; Lewis, et al., Transfusion 7 (1): 17-32, 1967; and Mazur, Science 168:939-949, 1970 for effects of cooling velocity on survival of marrow-stem cells and on their transplantation potential). The heat of fusion phase where water turns to ice should be kept to a minimum. The cooling procedure can be carried out by use of, e.g., a programmable freezing device or a methanol bath procedure known in the art.

A programmable freezing apparatus allows the determination of optimal cooling rates and facilitates standard reproducible cooling. A programmable controlled-rate freezer such as Cryomed or Planar permit tuning of the freezing regimen to the desired cooling rate curve. For example, for marrow cells in 10% DMSO and 20% plasma, the optimal rate is 1° C. to 3° C. per minute from 0° C. to −80° C. In a preferred embodiment, this cooling rate of 1° C. to 3° C. per minute from 0° C. to −80° C. can be used. The container holding the cells must be stable at cryogenic temperatures and allow for rapid heat transfer for effective control of both freezing and thawing. Sealed plastic vials (e.g., Nunc, the Wheaton Cryule®) or glass ampules can be used for multiple small amounts (1-2 mL) or larger amounts (e.g., 5 to 30 mL), while larger volumes (20 to 200 mL) can be frozen in polyolefin bags (e.g., Del-Med) or ethylene vinyl acetate freezer bags (e.g., OriGen Biomedical) held between metal plates for better heat transfer during cooling. By way of example, bags of bone marrow cells have been successfully frozen by placing them in −80° C. freezers which gives a cooling rate of approximately 3° C./minute.

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol, which is placed in a mechanical refrigerator (e.g., Harris or Revco) at −80° C. Thermocouple measurements of the methanol bath and the samples indicate the desired cooling rate of 1° C. to 3° C. per minute. After at least two hours, the specimens have reached a temperature of −80° C. and can be placed directly into liquid nitrogen (−196° C.) for permanent storage.

After thorough freezing, the NK cell composition and/or preparation can be rapidly transferred to a long-term cryogenic storage vessel. In a preferred embodiment, samples are cryogenically stored in liquid nitrogen (−196° C.) or its vapor (between about −140° C. and −180° C.). In another preferred embodiment, samples are cryogenically stored in liquid nitrogen vapor phase (e.g., at about −140° C. to −180° C.). Such storage is greatly facilitated by the availability of highly efficient liquid nitrogen refrigerators, which resemble large Thermos® containers with an extremely low vacuum and internal super insulation, such that heat leakage and nitrogen losses are minimized.

Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey and Linner, Nature 327:255, 1987; Linner et al., J. Histochem. Cytochem. 34 (9): 1123-1135, 1986; see also U.S. Pat. No. 4,199,022 by Senkan et al., U.S. Pat. No. 3,753,357 by Schwartz, U.S. Pat. No. 4,559,298 by Fahy).

Cryopreserved or frozen cells are preferably thawed quickly (e.g., in a water bath maintained at 37° C. to 41° C.), and chilled immediately upon thawing. In a specific embodiment, the vial containing the frozen cells can be immersed up to its neck in a warm water bath with gentle rotation to ensure mixing of the cell suspension as it thaws and to increase heat transfer from the warm water to the internal ice mass. As soon as the ice has completely melted, the vial is immediately placed in ice.

In an embodiment, a cryopreserved NK cell composition and/or preparation is thawed, and the full preparation, or a portion thereof, is infused into a human or animal patient in need thereof (e.g., having leukemia, such as AML; another hematological malignancy; a viral infection (e.g., HIV, HSV1 or 1, Hepatitis A, B, or C, zika, SARS-CoV or SARS-CoV-2, and the like); or other infection as disclosed herein). Several procedures relating to processing of the thawed cells are available, known in the art, and can be employed if desirable.

It can be desirable to treat the cells to prevent cellular clumping upon thawing. To prevent clumping, various procedures are well known in the art and can be used in the disclosed methods, including, but not limited to, the addition of DNase before and/or after freezing (Spitzer et al., Cancer 45:3075-3085, 1980), low molecular weight dextran and citrate, and/or hydroxyethyl starch (Stiff et al., Cryobiology 20:17-24, 1983), and the like.

The cryoprotective agent, if toxic in humans, should be removed prior to therapeutic use of the thawed NK cell composition and/or preparation. In an embodiment employing DMSO as the cryopreservative, it is preferable to omit this step to avoid cell loss. However, where removal of the cryoprotective agent is desired, the removal is preferably accomplished upon thawing.

For example, removal of the cryoprotective agent can be by dilution to achieve the cryoprotective agent at an insignificant concentration. This can be accomplished by addition of medium, followed by, if necessary, one or more cycles of centrifugation to pellet cells, removal of the supernatant, and resuspension of the cells. For example, intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, Biochemical Methods in Cell Culture and Virology, Dowden, Hutchinson & Ross, Stroudsburg, Pa., pp. 18-19, 1977; Methods in Medical Research, Eisen et al., eds., Vol. 10, Year Book Medical Publishers, Inc., Chicago, pp. 39-47, 1964) can be performed to confirm cell survival. The percentage of viable antigen (e.g., CD56) positive cells can be determined by calculating the number of antigen positive cells that exclude 7-AAD (or other suitable dye excluded by viable cells) in an aliquot of the cells, divided by the total number of nucleated cells (TNC) (both viable and non-viable) in the aliquot of the cells. The number of viable antigen positive cells can then be determined by multiplying the percentage of viable antigen positive cells by the TNC.

Prior to cryopreservation and/or after thawing, the total number of nucleated cells, or in a specific embodiment, the total number of CD56⁺ cells, can be determined. For example, total nucleated cell count can be performed by using a hemocytometer and exclusion of trypan blue dye. Specimens that are of high cellularity can be diluted to a concentration range appropriate for manual counting. Final cell counts for products are corrected for any dilution factors.

Total nucleated cell count equals viable nucleated cells per mL x volume of product in milliliters (mL). The number of CD56⁺ positive cells in the sample can be determined, e.g., by use of flow cytometry using anti-CD56 monoclonal antibodies conjugated to a fluorochrome.

The NK cell compositions and/or preparations can be used in immunotherapy for the treatment of solid tumors, hematopoietic malignancies, viral disorders, bacterial infections and the like. In some embodiments, the NK cell compositions and/or preparations are administered to inhibit tumor growth in a subject (also referred to as a patient) in need thereof. A method according to this aspect of the present invention is affected by administering a therapeutically effective amount of an NK cell composition and/or preparation to the subject. As used herein, “treating” or “treatment” includes, but is not limited to, the administration of an NK cell composition and/or preparation to reduce or delay the onset of the symptoms, complications, or biochemical indicia of a disease, alleviating the symptoms or arresting or inhibiting further development of the disease, condition, or disorder (e.g., cancer, metastatic cancer, metastatic solid tumors, viral or bacterial symptoms). Treatment can be prophylactic, i.e., as an adjuvant (to prevent relapse or delay the onset of the disease, or to prevent the manifestation of clinical or subclinical symptoms thereof) or therapeutic suppression or alleviation of symptoms after the manifestation of the disease.

In one embodiment, an NK cell composition and/or preparation is administered in an amount effective to reduce or eliminate a cancer, such as a solid tumor or a hematologic malignancy, such as for example, leukemia or lymphoma, and the like, or prevent its occurrence or recurrence. “An amount effective to reduce or eliminate the solid tumor or hematologic malignancy or to prevent its occurrence or recurrence” or “an amount effective to reduce or eliminate the hyperproliferative disease or to prevent its occurrence or recurrence” refers to an amount of an NK cell composition and/or preparation that improves a subject's outcome or survival following treatment for the tumor disease state or hyperproliferative disease as measured by patient test data, survival data, elevation or suppression of tumor marker levels, reduced susceptibility based upon genetic profile or exposure to environmental factors. “Inhibiting tumor growth” refers to reducing the size or viability or number of cells of a tumor. “Cancer,” “malignancy,” “solid tumor,” or “hyperproliferative disease” are used as synonymous terms and refer to any of a number of diseases that are characterized by uncontrolled, abnormal proliferation of cells; the ability of affected cells to spread locally or through the bloodstream and lymphatic system to other parts of the body (i.e., metastasize); as well as any other characteristic structural and/or molecular features. A “cancerous,” “malignant cell,” or “solid tumor cell” is understood as a cell having specific structural properties, lacking differentiation, and being capable of invasion and metastasis. “Cancer” refers to all types of cancer or neoplasm or malignant tumors found in mammals, including carcinomas and sarcomas. Examples are cancers of the breast, lung, non-small cell lung, stomach, brain, head and neck, medulloblastoma, bone, liver, colon, genitourinary, bladder, urinary, kidney, testes, uterus, ovary, cervix, or prostate, as well as melanoma, mesothelioma, and sarcoma, (see DeVita, et al., (eds.), Cancer Principles and Practice of Oncology, 6^{th}. Ed., Lippincott Williams & Wilkins, Philadelphia, Pa., 2001; this reference is herein incorporated by reference in its entirety for all purposes). “Hyperproliferative disease” refers to any disease or disorder in which the cells proliferate more rapidly than normal tissue growth. Thus, a hyperproliferating cell is a cell that is proliferating more rapidly than normal cells.

A “solid tumor” includes, but is not limited to, a sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, mucinous carcinoma, carcinoma muciparum, carcinoma mucocellulare, choriomeningitis carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, scirrhous carcinoma, carcinoma scroti, signet-ring cell carcinoma, carcinoma simplex, small-cell carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, acute myelogenous leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In another embodiment, there is provided a method of inhibiting a viral infection in a subject in need thereof. The method is affected by administering a therapeutic amount of an NK cell composition and/or preparation to a subject. Viral infections suitable for treatment with an NK cell composition and/or preparation include, but are not limited to, HIV, lymphatic choriomenengitis virus (LCMV), cytomegalovirus (CMV), vaccinia virus, coronavirus (SARS-CoV and SARS-CoV-2), influenza and para-influenza virus, zika virus, hepatitis (including hepatitis A, hepatitis B, hepatitis C, non-A-non-B, and the like), herpes simplex virus (HSV 1 or HSV 2), herpes zoster virus, and Theiler's virus. Other infectious diseases suitable for treatment with an NK cell composition and/or preparation include, but are not limited to, parasitic infections such as Plasmodium, Leishmania and Toxoplasma infections, and bacterial infections such as mycobacteria and Listeria (for a review of NK cells in treatment of viral, bacterial and protozoan diseases see Zucchini et al., Exp. Rev. Anti-Infect. Ther. 6:867-885, 2008, which reference is incorporated by reference herein).

According to some aspects of the present disclosure, there are provided pharmaceutical compositions comprising an NK cell preparation for the treatment of disease. For example, the NK cell preparation is formulated together with a pharmaceutically acceptable carrier to form an NK cell composition for the treatment of e.g., metastatic cancer, solid tumors, hematological malignancy, hyperproliferative disease, any viral infection, a bacterial infection, and the like.

Therapeutic applications, compositions, or medicaments are administered to a subject suspected of, or already suffering from, such a disease, and are provided in an amount sufficient to cure or partially arrest the symptoms of the disease (biochemical, histologic and/or behavioral), including its complications and intermediate pathological phenotypes in development of the disease. An amount adequate to accomplish such therapeutic treatment is defined as a therapeutically effective dose. In therapeutic regimes, NK cell compositions and/or preparations are usually administered in several dosages until a sufficient anti-proliferative response has been achieved. Typically, the anti-proliferative response is monitored, and repeated dosages are given if the anti-proliferative response starts to wane.

Effective doses of an NK cell composition and/or preparation for the treatment of disease, e.g., metastatic cancer, solid tumors, hematologic malignancy, a hyperproliferative disease, any viral infection, a bacterial infection, and the like, as described herein, vary depending upon factors including means of administration, target site, physiological state of the patient, whether the patient is human or an animal, other medications administered, and whether treatment is prophylactic or therapeutic. Usually, the patient is a human, but nonhuman mammals including transgenic mammals can also be treated. Treatment dosages need to be titrated to optimize safety and efficacy.

For administration with a therapeutic NK cell composition and/or preparation, the dosage typically ranges from about 50 to about 500 million total cells per dose. In some embodiments, a dosage is about 50, 100, 200, 300, 400 or 400 million cells per dose. In some embodiments, a dose of the NK cell composition and/or preparation can be from about 50 million to about 2 billion viable cells per dose. In some embodiments, a dose of the NK cell composition and/or preparation contains about 100 million, about 200 million, about 300 million, about 400 million, about 500 million, about 600 million, about 750 million, about 1 billion, about 1.5 billion or about 2 billion viable cells. In some embodiments, a dose of the NK cell composition and/or preparation can be from about 50 million to about 2 billion viable CD56⁺ cells per dose. In some embodiments, a dose of the NK cell composition and/or preparation contains about 100 million, about 200 million, about 300 million, about 400 million, about 500 million, about 600 million, about 750 million, about 1 billion, about 1.5 billion or about 2 billion viable CD56⁺ cells.

An exemplary treatment regimen entails administration about once, twice, or even three times per week, once, twice or even three times per every two weeks, about once per month, about once every 3 to 6 months, or about once every 6 to 12 months. Multiple administrations of NK cell compositions and/or preparations can be provided. Intervals between single dosages can be a few days, weekly, monthly, yearly, or some combination of such time periods. Intervals can also be irregular and as indicated by measuring blood levels of the NK cell composition and/or preparation in the patient. Alternatively, the NK cell compositions and/or preparations can be administered as a sustained release formulation, in which case less frequent administration is required. Dosage and frequency vary depending on the half-life of the NK cell compositions and/or preparations in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the remainder of their lives. In therapeutic applications, a relatively high dosage at relatively short intervals is sometimes required until progression of the disease is reduced or terminated, and preferably until the patient shows partial or complete amelioration of disease symptoms or disease indication. Thereafter, the patent can be administered a prophylactic regimen.

Compositions of an NK cell composition and/or preparation for the treatment of disease, e.g., metastatic cancer, solid tumors, hematological malignancy, a hyperproliferative disease, any viral infection, a bacterial infection, and the like, can be administered by intravenous, intravesicular, intraarterial, intracranial, or intraperitoneal means.

In some embodiments, an NK cell composition and/or preparation is administered after a chemotherapy regimen. The chemotherapy regimen can be a single agent or multi-agent regimen. In some embodiments, the chemotherapy regimen is an induction regimen or a consolidation regimen. In some embodiments, the chemotherapy regimen is a salvage regimen.

In some embodiments, a fixed dose of an NK cell composition and/or preparation can be administered following chemotherapy regimen or a cycle thereof, such as an induction regimen. A fixed dose of an NK cell composition and/or preparation can also be administered following a consolidation regimen or a cycle thereof. A fixed dose of an NK cell composition and/or preparation can also be administered following a salvage regimen or a cycle thereof. In some embodiments, a fixed dose of an NK cell composition and/or preparation can be administered following a second induction regimen or cycle thereof, or a second cycle of an induction regimen, if desired or necessary. In some embodiments, a fixed dose of an NK cell composition and/or preparation can be administered following a second consolidation regimen or cycle thereof, or a second cycle of a consolidation regimen is desired or necessary. In some embodiments, a fixed dose of an NK cell composition and/or preparation can be administered following a second salvage regimen or cycle thereof, or a second cycle of a salvage regimen is desired or necessary.

### EXAMPLES

The present invention is not to be limited in scope by the specific embodiments described herein. Indeed, various modifications of the invention, in addition to those described herein, will be apparent to those skilled in the art from the descriptions herein and accompanying figures. Such modifications are intended to fall within the scope of the appended claims.

Various publications, including patents, patent application publications, and scientific literature, are cited herein, the disclosures of which are incorporated by reference in their entireties for all purposes. While illustrative embodiments have been depicted and described, it will be appreciated that various changes can be made herein without departing from the spirit and scope of the invention.

### Example 1: Generation of an NK Cell Composition and/or Preparation

This Example describes the production and storage of an NK cell preparation and its formulation into a NK cell composition.

Umbilical cord blood/placental blood unit(s) (CBU) were collected from human donors at birth. Typically, the CBUs are frozen after collection and the selected CBUs are donor eligible and fully qualified prior to use. The collected blood was then mixed with an anti-coagulant to prevent clotting. The blood was stored under quarantine at 4° C. in a monitored refrigerator. The received units were assessed, and the units to be processed for expansion were determined.

Phase 1 involved expansion of hematopoietic stem and progenitor cells. CD34⁺ enriched hematopoietic stem and progenitor cells (HSPC) were seeded into tissue culture treated plastic vessels (Thermo Scientific Nunc™ EasYFlask™). The enriched CD34⁺ HSPCs were prepared from pooled cord blood units as described in US Patent Application Publication No. 2013/0095079 (incorporated herein by reference). Generally, CD34⁺ cells from at least 4 cord blood units were pooled, either before or after enrichment for CD34⁺ cells.

The tissue culture treated plastic vessels were pre-coated with a solution of recombinant human fibronectin fragment (Takara Bio, Inc.; RetroNectin® Recombinant Human Fibronectin Fragment, 5 μg/mL and 0.8 μg/cm² surface area) and a Notch agonist (Delta1^{extIgG}, 2.5 μg/mL and 0.4 μg/cm² surface area) in DPBS (Gibco). Culture medium consisted of a serum free cell culture medium designed and suitable for the culture and expansion of hematopoietic cells (StemSpan™ Serum-Free Expansion Medium II (SFEM II; StemCell Technologies)) supplemented with 50 ng/mL each of rhSCF (Miltenyi Biotec), Flt-3L (Miltenyi Biotec), TPO (Miltenyi Biotec), and IL-6 (Miltenyi Biotec), and 10 ng/mL IL-3 (Miltenyi Biotec). Cells were cultured for 14 days, passaging the cells into larger volume culture vessels when appropriate to maintain a cell density <2×10⁶ cells/mL.

An alternate protocol for the Phase 1 expansion culture that produces the same cell preparation includes the replacement of Delta1^{extIgG} as the Notch agonist with 2.5 to 10 μg/mL of a purified anti-human Notch 1 antibody (BioLegend, LEAF™ Purified, clone MHN1-519).

Phase 2 of the culturing process differentiates the expanded hematopoietic stem and progenitor cells toward an NK cell phenotype. At the end of the 14-day expansion phase (above), all cells were collected and re-plated into tissue culture treated plastic vessels without the Notch agonist (Delta1^{extIgG} or RetroNectin®) coating. The culture medium comprises RPMI 1640 (Gibco) supplemented with 5 to 10% heat inactivated fetal bovine serum (FBS; Gibco), 40 ng/mL rhIL-15 (PeproTech), and 50 U/mL rhIL-2 (PeproTech). Cells were cultured for up to 14 additional days, passaging the cells into larger volume culture vessels when appropriate to maintain a cell density <2×10⁶ cells/mL.

In addition to CD56, the CD56⁺ cells expressed high a frequency of NKp30, NKp46, NKp44, NKG2A, and granzyme B, moderate to a high frequency of perforin and CD107a, a low to moderate frequency of NKG2D and substantially no KIRs. In those batches tested the CD56⁺ cells were found to be KIR⁻. (As used herein, “KIR⁻” refers to KIR2DL1, KIR2DS1, KIR2DS3, KIR2DS5, KIR2DL2, KIR2DL3, KIR2DS2, KIR2DS4, KIR3DL1, and KIR3DS1.) CD16 was expressed at a low to moderate frequency. In those batches tested, the CD56⁻ cells expressed a moderate to high frequency of granzyme B, a high frequency of CD107a and a low frequency of perforin. See Table 1 above. Table 2, also above, shows the phenotypic attributes of the CD56⁺ cells in an exemplary NK cell preparation.

The NK cell preparation also included approximately 25% to 50% CD56⁻ cells. These cells were largely myeloid derived and can include dendritic cells, macrophages and granulocytes. Across the CD56⁻ cells in the NK cell preparation, the following cell surface markers were expressed at the indicated frequency: Granzyme B is expressed at intermediate frequency and CD107a is expressed at high frequency. Table 3, above, shows the phenotypic attributes of the CD56⁻ cells in an exemplary NK cell preparation.

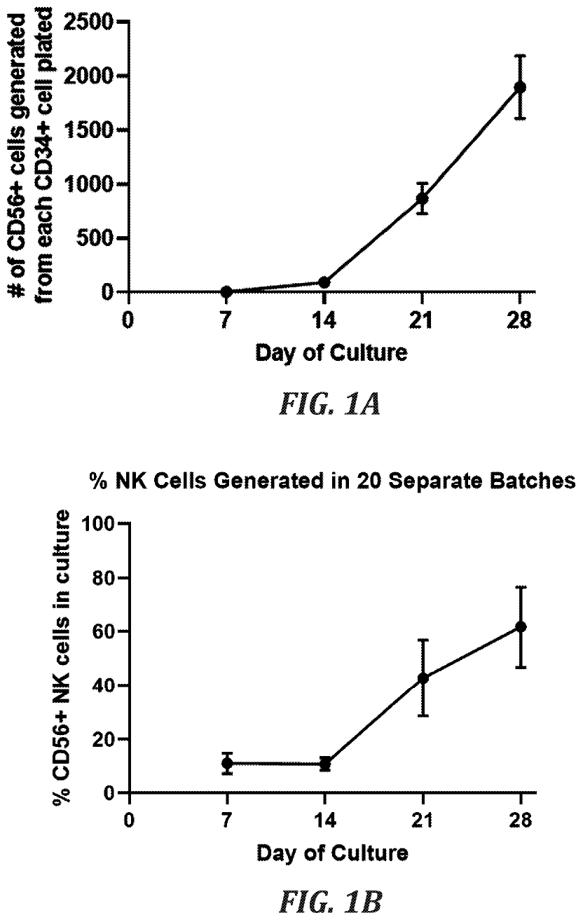

FIG. 1A shows the increase in CD56⁺ cells/starting CD34⁺ cells over the course of a 28-day culture (phases 1 (expansion) and 2 (differentiation)). FIG. 1B demonstrates reproducibility of CD56⁺ cell generation over a 28-day cell culture process. FIGS. 1A and 1B show the results from 20 batches of NK cell preparations produced following the process described above.

### Example 2: Production of an NK Cell Preparation without Animal Derived Serum

To avoid the use of animal derived serum, an alternate protocol for the Phase 2 culture (differentiation) to produce an NK cell preparation involved the replacement of fetal bovine serum (FBS) with heat inactivated human AB serum (Valley Biomedical) or with human platelet lysate (Mill Creek Life Sciences PLTMax®). Applicant unexpectedly was able to substitute heat inactivated human AB serum or human platelet lysate in phase 2 (the differentiation phase) of the process described in Example 1.

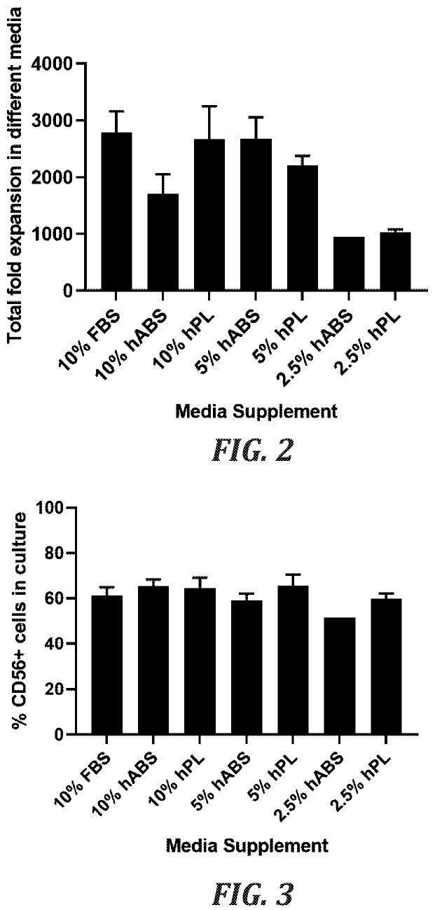

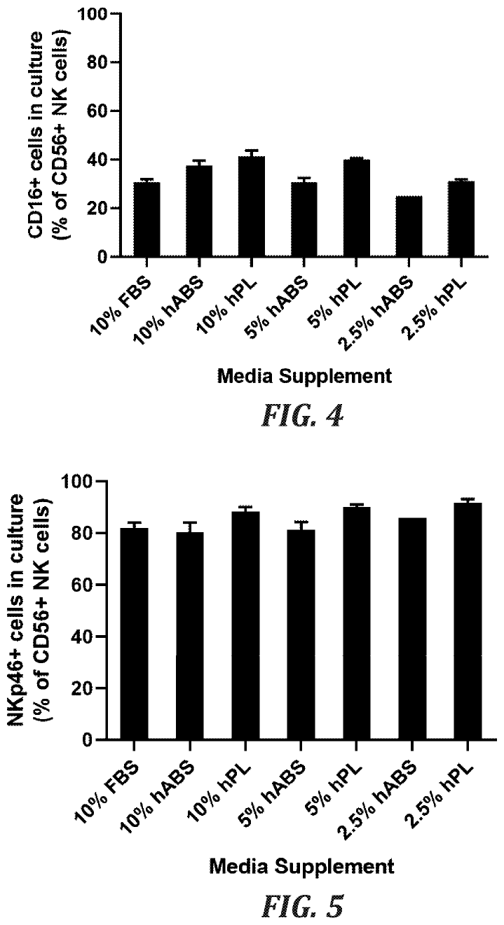

Replacement of FBS with human AB serum (hABS) or with human platelet lysate (hPL) had minimal effects on the cell expansion during differentiation (FIG. 2) or the number of CD56⁺ cells produced (FIG. 3). hPL maintained expansion of the culture slightly better than hABS, but the resulting cell phenotypes are very similar among the FBS, hABS, and hPL cultures.

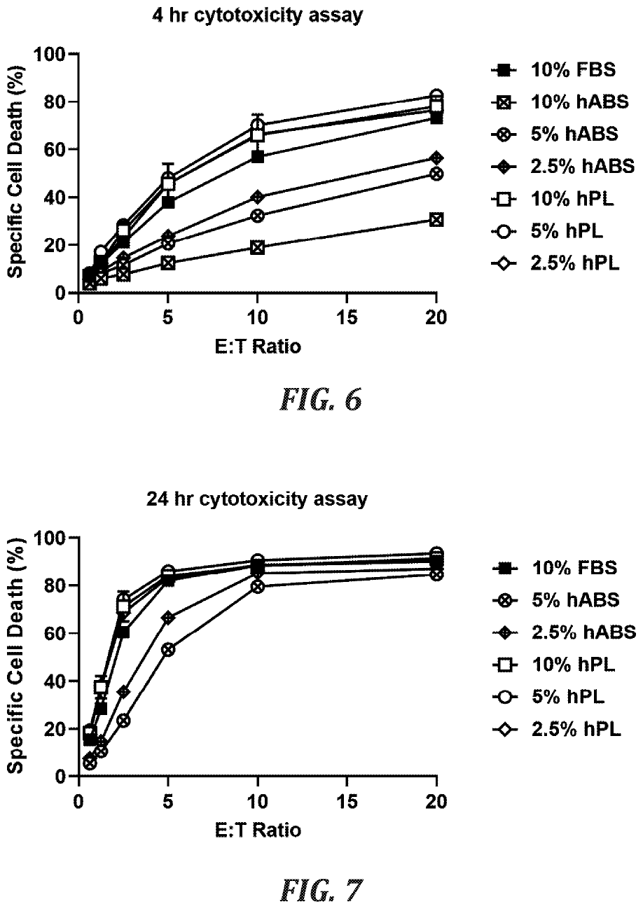

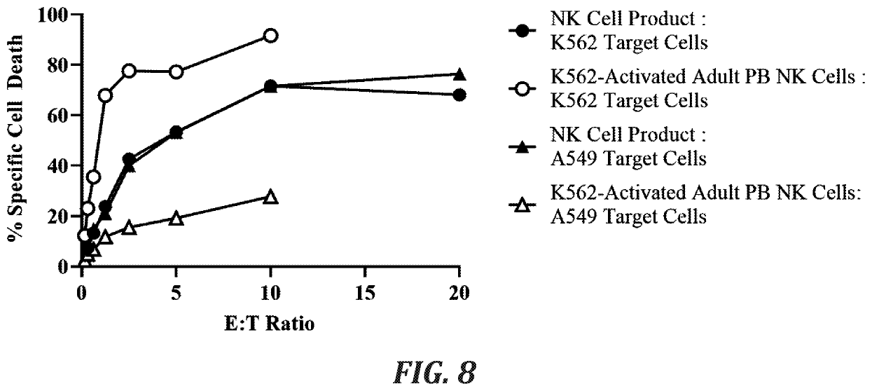

FIGS. 4 and 5 show the percent CD16⁺ cells (FIG. 4) and the percent NKp46⁺ cells (FIG. 5), both as a percent of CD56⁺ cells, on day 28 of culture comparing different serum supplements used in Phase 2 of the culture.

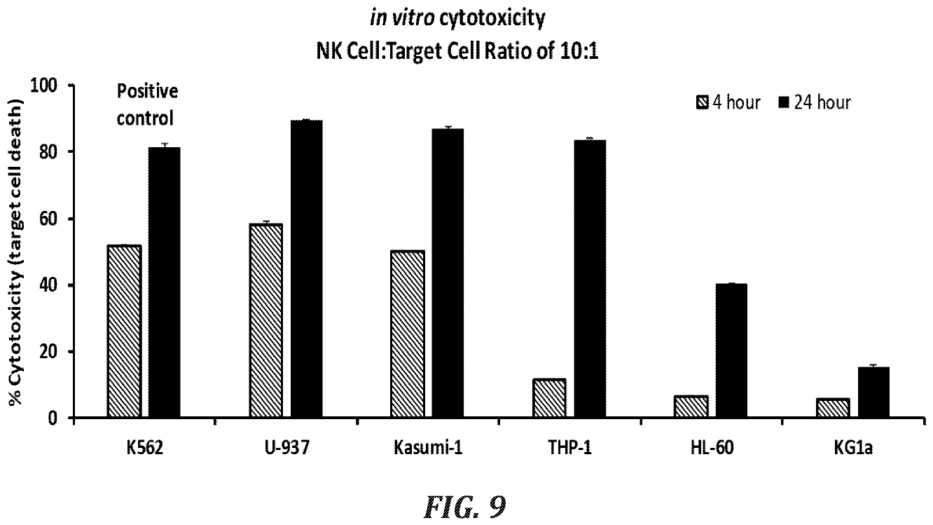

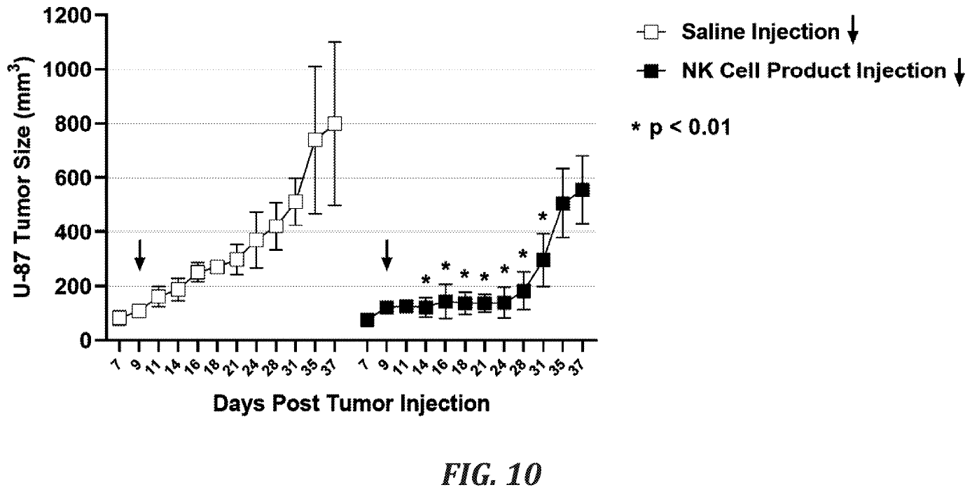

In vitro cytotoxicity analysis using an NK cell preparation, and K562 target cells, was undertaken to compare NK cell preparations cultured with the various serum supplements in Phase 2 (FBS, hABS, or hPL). FIGS. 6 and 7 show cultures supplemented with hPL developed a more cytotoxic NK cell population than those supplemented with hABS.

### Example 3: In Vitro Cytotoxicity Assay with an NK Cell Preparation

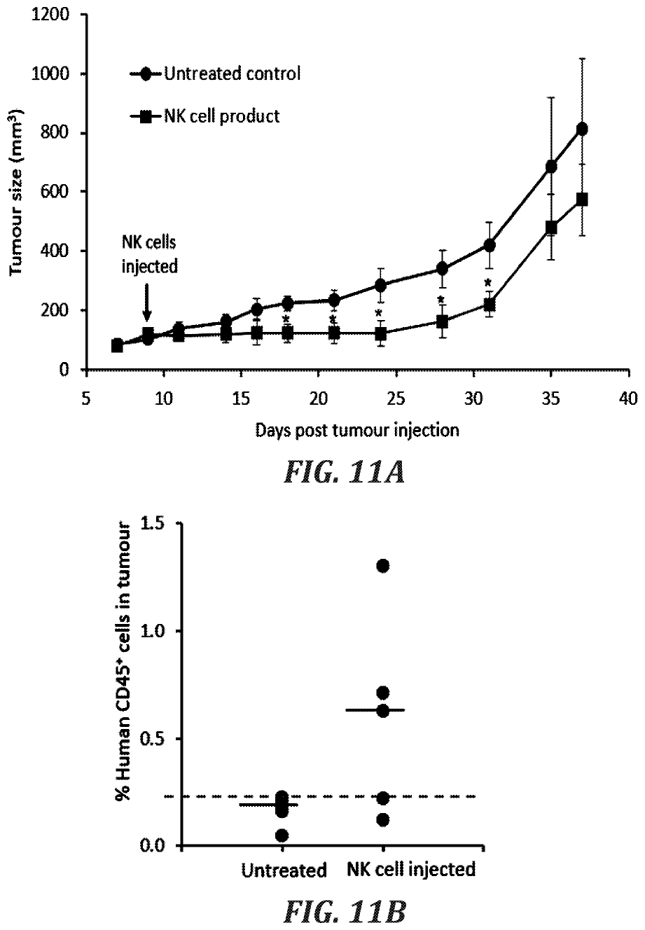

An in vitro cytotoxicity assay was performed using the Day 28 NK cell preparation, or K562-activated adult peripheral blood NK cells, as effector cells (E). The NK cell preparation was prepared as described above. Fluorescently labeled K562 cells (a chronic myelogenous leukemia cell line) or A549 cells (a lung carcinoma cell line) were included as target cells (T). The cells were co-incubated at the E:T ratios indicated in FIG. 8 for 4 hours at 37° C. Cells were then labeled with 4′,6-diamidino-2-phenylindole (DAPI), and the fluorescent target cells that co-labeled with DAPI identified as dying and dead target cells by flow cytometric analysis.

FIG. 8 shows the NK cell preparation (referred to as NK Cell Product) was active against K562 and A549 target cells with similar activity (curves indicated with filled circles and filled triangles). In contrast, adult peripheral blood NK cells, activated with K562 cells (referred to as K562-Activated Adult PB NK Cells), were active against K562 target cells (top curve with open circles), but not against A549 target cells (bottom line with open triangles). This study demonstrated the ability of the NK cell preparation to be active against various target cells.

### Example 4: Additional In Vitro Assays with an NK Cell Preparation

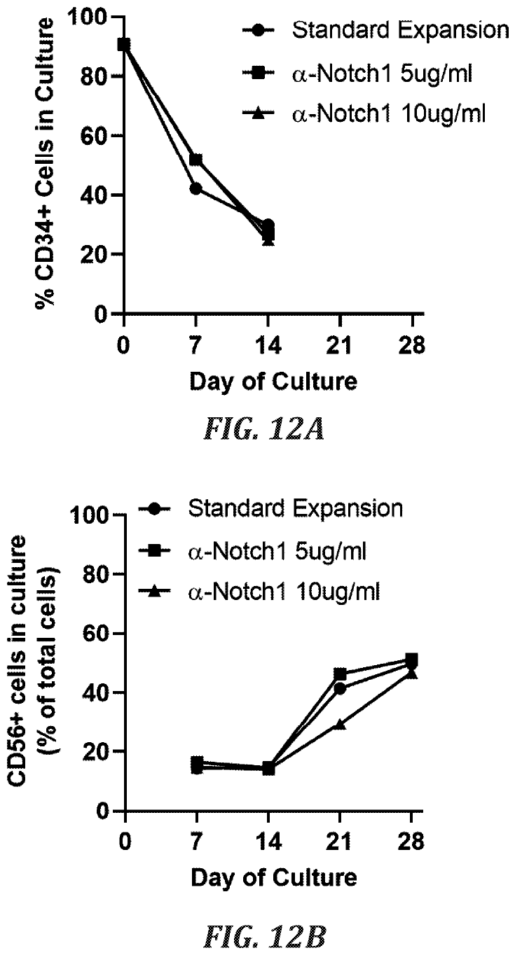

The cytotoxicity of the NK cell preparation was tested in a standard in vitro cell killing assay (as described above in Example 3) against a range of myeloid cell leukemia (similar to AML) cell lines. FIG. 9 demonstrates the NK cell preparation exhibited cell killing against many of these cancer cell lines. In FIG. 9, for each cell line, the left bar indicates cytotoxicity at 4 hours and the right bar indicates cytotoxicity at 24 hours. The K562 cell line is an accepted standard for NK cell activity and serves as a positive control.

### Example 5: In Vivo Mouse Xenograft Assay with an NK Cell Preparation

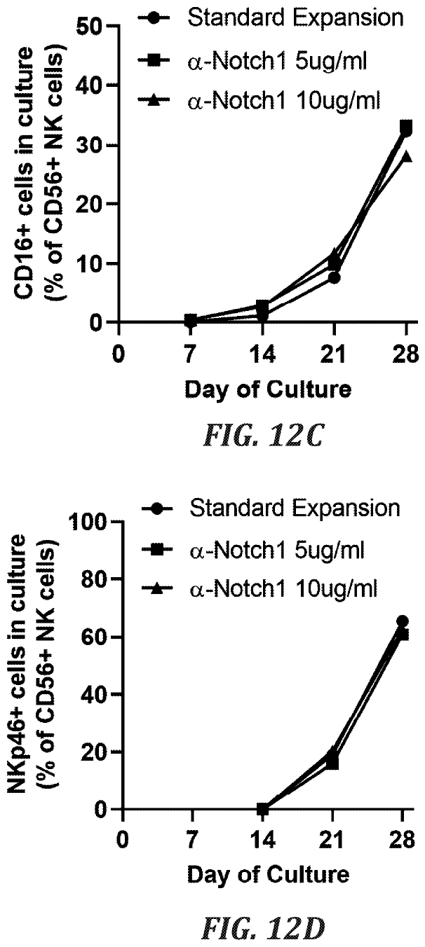

The activity of an NK cell preparation in a U-87 glioblastoma tumor model was tested in NSG mice with or without NK cell preparation treatment, by measuring U-87 tumor burden. U-87 cells were subcutaneously injected with Matrigel® (a gelatinous protein mixture secreted by Engelbreth-Holm-Swarm (EHS) mouse sarcoma cells intended to resemble the complex extracellular environment found in many tissues and as a substrate for culturing cells) into the rear flank of test NSG mice (2×10⁶ cells/mouse) and allowed to grow for 9 days to form a measurable tumor prior to intra-tumoral injection of 1×10⁷ cells of an NK cell preparation per mouse (referred to as NK Cell Product or Composition). Tumor growth was assessed 2 to 3 times/week by caliper measurement. FIG. 10 demonstrates the NK Cell Product was able to reduce tumor burden, as compared to the saline injection control.

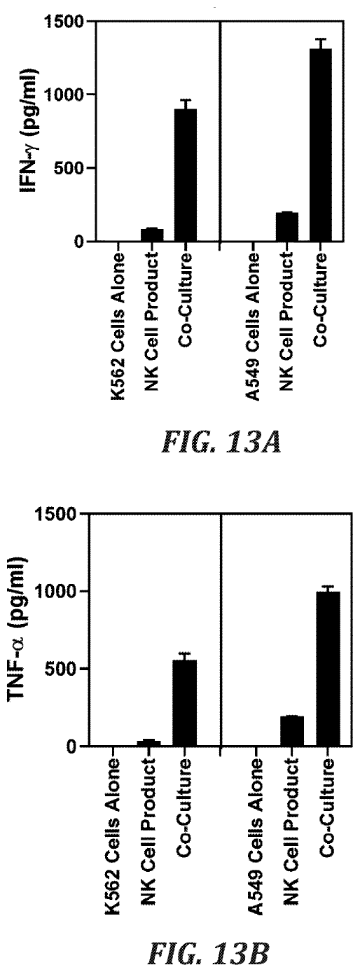

FIG. 11 shows a graph of the tumor size for the untreated control (line with circles) and for the NK cell preparation (line with squares) over time for this study. The NK cell preparation was able to cause a significant delay in tumor progression (FIG. 11). The NK cell preparation persisted and was detectable 37-days post injection, as measured by detecting human CD45⁺ cells in the tumor (FIG. 11).

### Example 6: Expansion of CD34⁺ HSPCs Using Notch 1 or Notch 2 Specific Antibodies

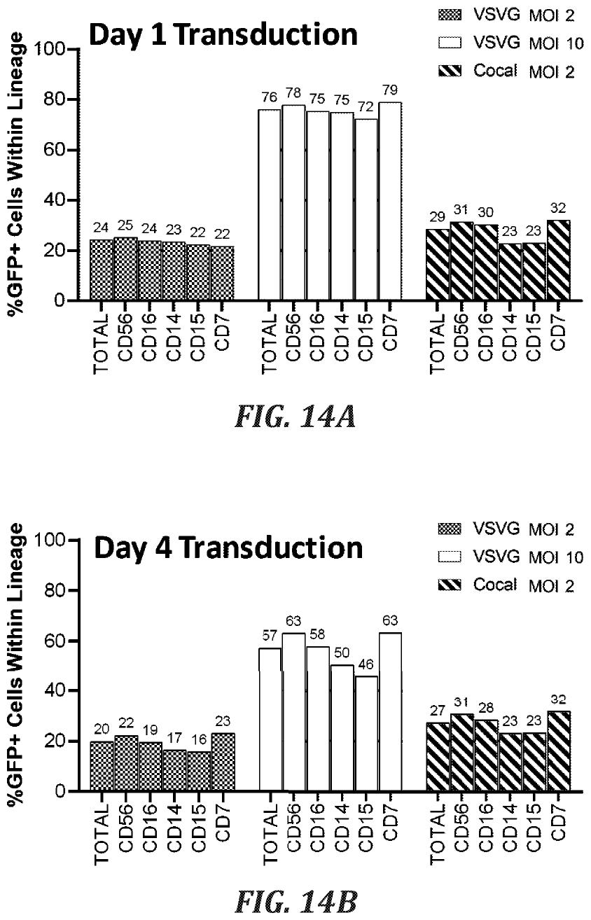

CD34⁺ HSPCs were expanded as described above (Example 1) on standard substrate (5 μg/mL RetroNectin®+2.5 g/mL DXI) or on 5 μg/mL RetroNectin®+5 g/mL anti-Notch1 antibody (anti-Notch 1 Ab) or on 5 μg/mL RetroNectin®+10 μg/mL anti-Notch1 Ab. FIGS. 12A-D show expansion of HSPCs with Notch 1 or Notch 2 antibodies (see US Patent Application Publication No. 2017/0107493, incorporated herein by reference) was comparable to expansion using DXI.

### Example 7: Cytokine Release by an NK Cell Preparation in the Presence of Tumor Cells

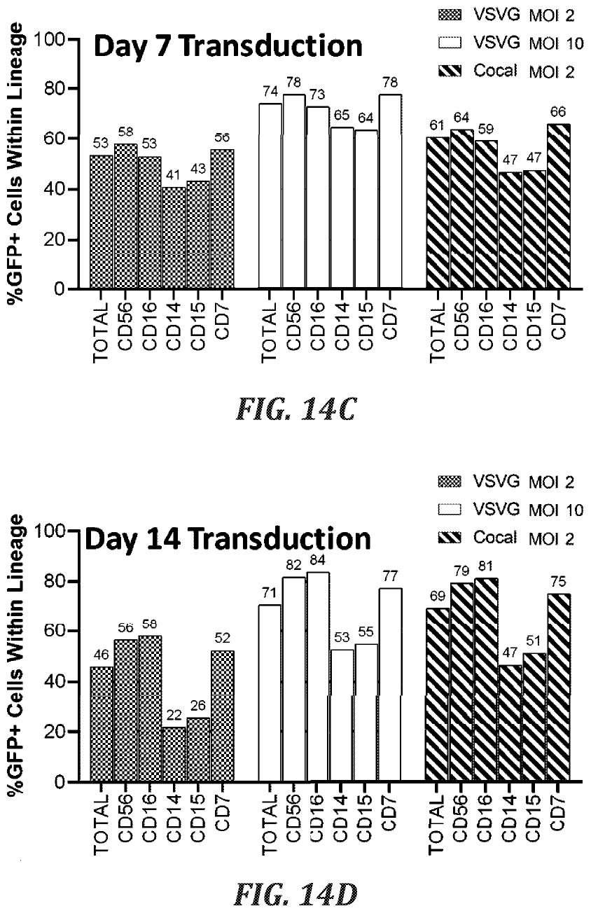

This study was performed to determine the expression of the cytokines interferon gamma (IFNγ) or tumor necrosis factor alpha (TNFα) by an NK cell preparation in the presence and absence of K562 or A549 tumor cells. FIG. 13 shows an NK cell preparation produced by the above methods (referred to as an NK Cell Product) or K562 or A549 tumor cells were cultured alone, or together for three days. The study was run in triplicate. After three days, cytokine levels were determined by Luminex® assay. (The limit of detection was 1 μg/mL of cytokine.) As shown in FIG. 13, both IFNγ (FIG. 13A) and TNFα (FIG. 13B) levels increased when an NK cell preparation was co-cultured in the presence of tumor cells. These results indicated that the NK cells in the preparation became more active in the presence of tumor cells.

### Example 8: Transduction Time Course for Production of Genetically Modified NK Cell Preparations

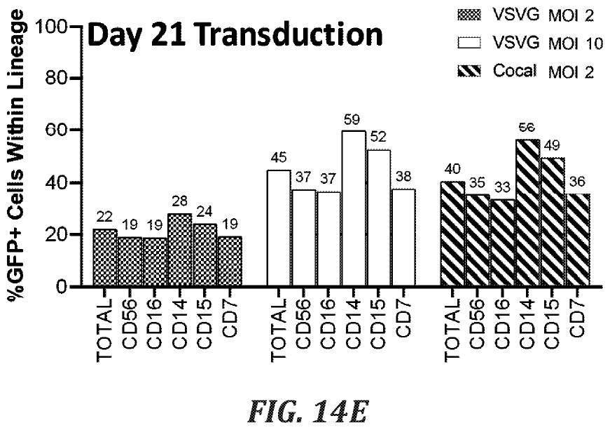

This study was performed to determine suitable times during the expansion and differentiation phases for genetic modification. GFP (green fluorescent protein) lentivirus with either VSVG (vesicular stomatitis virus) or Cocal pseudotyped virus envelope glycoprotein was used to transduce CD34⁺ cord blood cells using 2 different multiplicities of infection (MOIs) at different time points during the expansion phase (day 1, 4, 7, or 14) or the NK differentiation phase (day 21) and continued through 28 days of the NK cell culture. On day 28 cells were immunophenotyped for different cell populations including CD56, CD16, CD14, CD15, and CD7. FIG. 14 shows more consistent distribution across cell lineages was observed following transduction on day 1, 4, or 7.

### Example 9: Preparation of CAR Constructs Targeting Human CD19 Expressing Cells

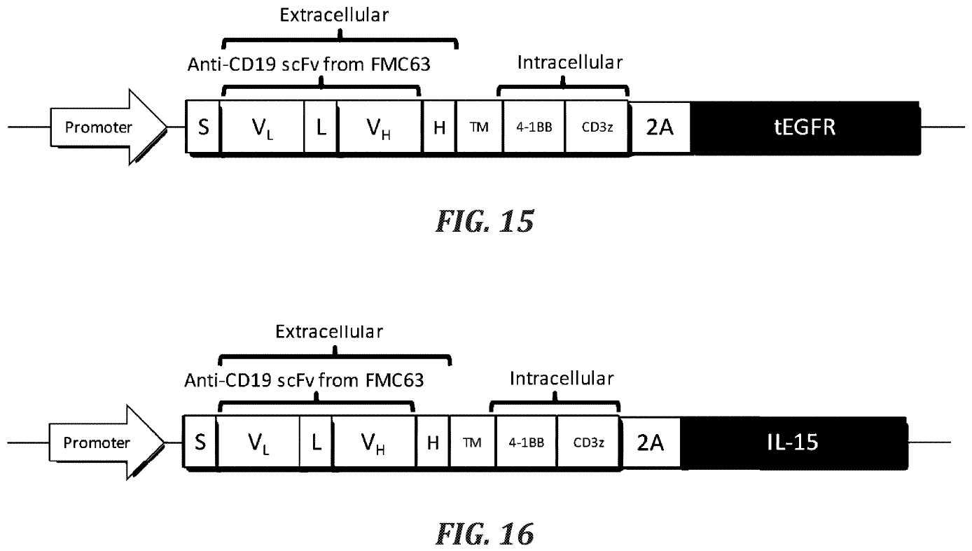

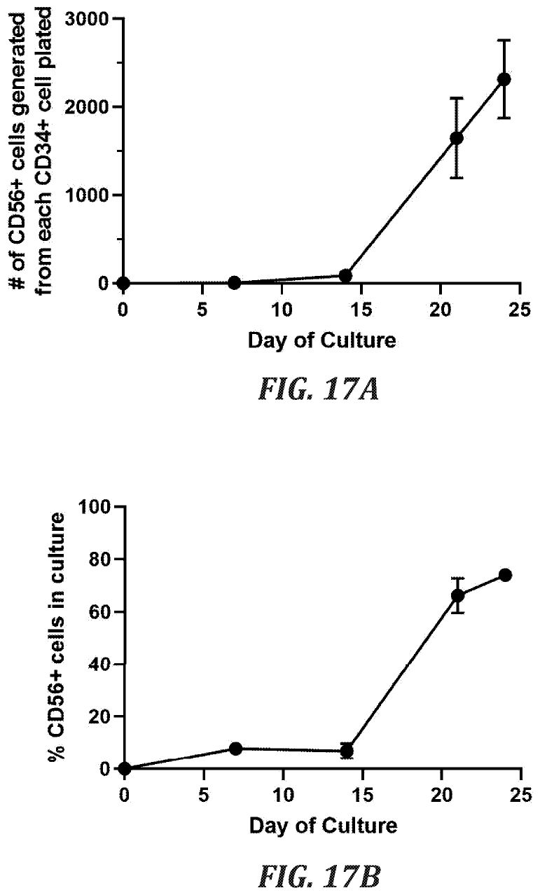

CAR constructs containing an anti-CD19 scFV (FMC63), a hinge region from human IgG₄, a CD28 transmembrane domain, a 4-1BB co-stimulatory domain and a CD3zeta intracellular signaling domain were prepared. The expression constructs include either tEGFR as a selectable marker (FIG. 15) or human IL-15 (FIG. 16). The CAR constructs were transduced into HSPCs, generally as described in Example 8.

### Example 10: Preparation and Characterization of NK Cells with IL-15 Priming During the Expansion Phase

This Example describes the production and characterization of an NK cell preparation with IL-15 added during the Phase 1 expansion stage.

Phase 1 involved expansion of hematopoietic stem and progenitor cells. CD34⁺ enriched hematopoietic stem and progenitor cells (HSPC) were seeded into tissue culture treated plastic vessels (Thermo Scientific Nunc™ EasYFlask™). The enriched CD34⁺ HSPCs were prepared as described above. The tissue culture treated plastic vessels were pre-coated with a solution of a recombinant human fibronectin fragment (Takara Bio, Inc.; RetroNectin®) Recombinant Human Fibronectin Fragment, 5 μg/mL and 0.8 μg/cm² surface area) and a Notch agonist (Delta1^{extIgG}; 2.5 μg/mL and 0.4 μg/cm² surface area) in DPBS (Gibco). Culture medium comprised a cell culture medium suitable and adapted for the culture and expansion of hematopoietic cells (StemSpan™ Serum-Free Expansion Medium II (SFEM II; StemCell Technologies)) supplemented with 50 ng/ml each of rhSCF (Miltenyi Biotec), Flt-3L (Miltenyi Biotec), TPO (Miltenyi Biotec), and IL-6 (Miltenyi Biotec), and 10 ng/mL IL-3 (Miltenyi Biotec). Cells were cultured for 14 days, passaging the cells into larger volume culture vessels when appropriate to maintain a cell density <2×10⁶ cells/mL. The expansion culture was primed by the addition of IL-15 (Pepro Tech) at 40 ng/ml from day 7 to 14 or day 10 to 14.

Phase 2 of the culturing process differentiates the expanded hematopoietic stem and progenitor cells toward an NK cell phenotype. At the end of the 14-day expansion phase (Phase 1 above), all cells were collected and re-plated at a cell density of 1×10⁶ cells/mL in 1000 mL culture medium into culture vessels (G-Rex®100M vessels, WilsonWolf) without a Notch agonist (Delta1^{extIgG} or RetroNectin®) coating. The culture medium comprised RPMI 1640 (Gibco) supplemented with 5% human platelet lysate (hPL; Mill Creek Life Sciences), 40 ng/mL rhIL-15 (PeproTech), and 50 U/mL rhIL-2 (PeproTech). Cells were cultured for 10 additional days, replacing 50% of the medium in the vessel with fresh differentiation medium 7 days into differentiation.

In addition to CD56, the CD56⁺ cells expressed a high frequency of NKp30, NKp46, NKp44, and NKG2A, and a moderate frequency of CD16, NKp44, and NKG2D.

Table 4 shows the phenotypic attributes of the CD56⁺ cell in an exemplary NK cell preparation produced by the method.

Table 4
Phenotypic attributes of CD56⁺ Cells

| CD56⁺ Cells | Frequency (Mean ± SD) |
|---|---|
| CD56+ population | 74.03 ± 1.40 |

Frequency within CD56⁺ population

| | |
|---|---|
| CD16⁺ | 35.21 ± 3.62 |
| NKp30⁺ | 71.29 ± 23.3 |
| NKp44⁺ | 25.39 ± 3.47 |
| NKp46⁺ | 84.31 ± 8.16 |
| NKG2A⁺ | 63.86 ± 4.49 |
| NKG2D⁺ | 50.01 ± 16.78 |

The NK cell preparation also included approximately 26% CD56⁻ cells. As with the prior method above, these cells are largely myeloid derived and can include dendritic cells, macrophages, and granulocytes. Table 5 shows the phenotypic attributes of the CD56⁻ cells in an exemplary NK cell preparation produced by this method.

TABLE 5
Phenotypic attributes of CD56⁻ cells

| CD56⁻ Cells | Frequency (Mean ± SD) |
|---|---|
| CD14⁻CD15⁺ | 6.46 ± 1.26 |
| CD14⁺CD15⁺ | 6.96 ± 1.19 |
| CD14⁺CD15⁻ | 0.55 ± 0.19 |
| CD14⁻CD15⁻ | (cell subsets below) |
| CD11b⁺CD11c⁺ | 3.67 ± 1.08 |
| HLA⁻DR⁺ | 4.06 ± 0.65 |
| CD7⁺ | 0.50 ± 0.14 |
| CD33⁺ | 1.83 ± 0.81 |
| CD34⁺ | 0.13 ± 0.14 |
| unidentified | 0.61 ± 0.21 |

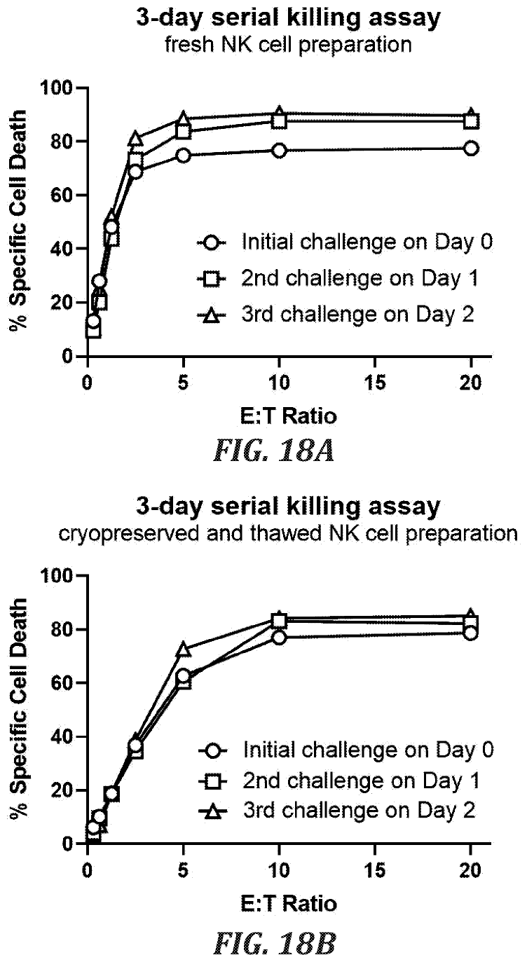

FIGS. 17A and 17B show the results of 4 batches of NK cell preparations produced at manufacturing scale following the method described above. FIG. 17A shows the increase in CD56⁺ cells/starting CD34⁺ cells over the course of a 24-day culture (phases 1 and 2). FIG. 17B demonstrates the reproducibility of CD56⁺ cell generation over the 24-day cell culture process.

### Example 11: In Vitro Cell Killing by an NK Cell Preparation Produced with IL-15 Priming

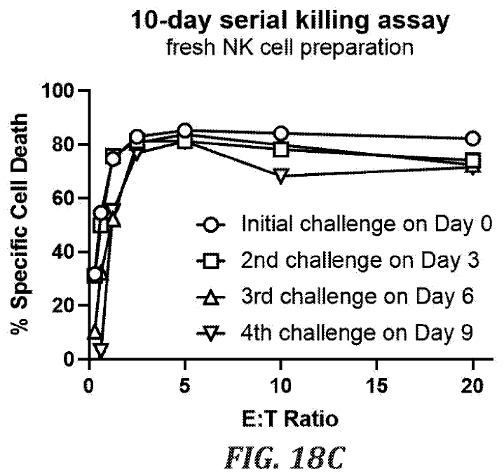

In this Example the capacity of the NK cell preparations produced with IL-15 priming to serially kill repeat doses of target cells is demonstrated. The serial killing assay was run over a prolonged duration in a modification of the standard in vitro cell killing assay described in Example 3. In this example, the cell killing assay used a Kasumi-1 myeloid cell leukemia cell line (similar to AML). Fresh NK cell preparations (FIG. 18A) or cryopreserved and thawed NK cell preparations (FIG. 18B) were plated into the assay at its initiation and received repeat doses of target cells every 24 hours for a total of three days, with analysis for specific cell death of the target cells performed 24 hours after each challenge. An additional assay was plated using a fresh NK cell preparation that received repeat doses of target cells every 3 days for a total of 10 days (FIG. 18C), with analysis for specific cell death of the target cells performed 24 hours after each challenge. Both the fresh NK cell preparation and the cryopreserved and thawed NK cell preparation achieved dose-dependent cell killing against repeat target additions over the duration of each experiment, demonstrating the ability of the NK cells to serially kill target cells for at least 10 days.

### Example 12: In Vivo Mouse AML Xenograft Assay with an NK Cell Preparation

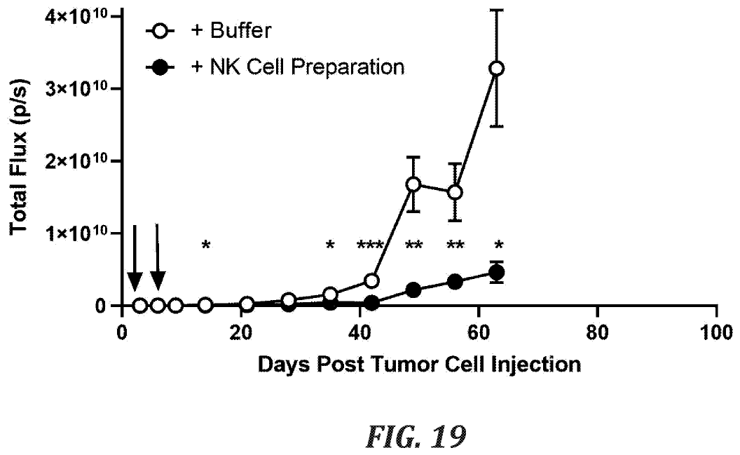

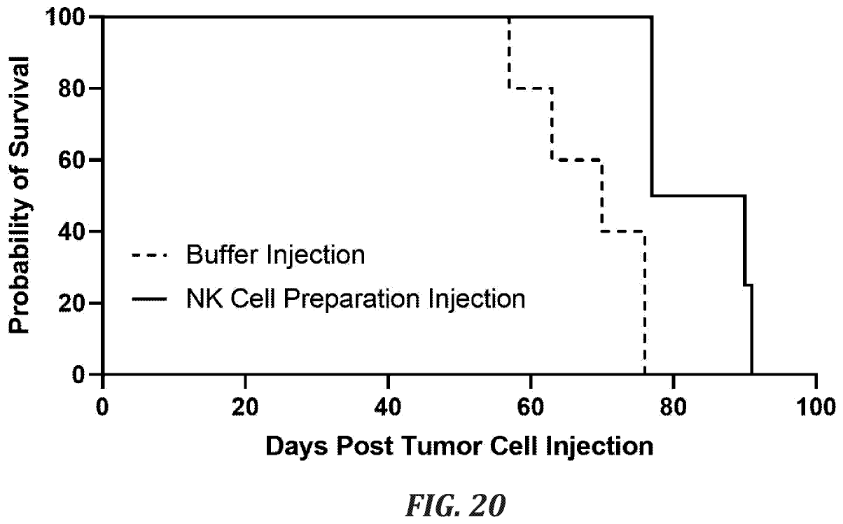

The activity of the NK cell preparation in the Kasumi-1 AML diffuse tumor model was tested in NSG mice with or without NK cell preparation treatment by measuring Kasumi-1 tumor burden and animal survival. Firefly-luciferase expressing Kasumi-1 cells were intravenously injected into the tail vein of NSG mice (2×10⁶ cells/mouse) on Day 0 prior to intravenous injection of two doses of 2×10⁷ cells of an NK cell preparation per mouse on Day 2 and Day 6. Tumor growth was assessed 1 to 2 times per week by bioluminescence imaging. Compared against buffer-injected control treatment, the NK cell preparation inhibited tumor progression (FIG. 19, p values: *=<0.05, **=<0.01, ***=<0.001) and significantly prolonged median survival in mice. (FIG. 20; 83.5 days versus 70 days, p=<0.01 by log-rank test).

### Example 13: Generation of Mesothelin CAR NK Cells-Transduction During the Expansion Phase

On Day 7 of Phase 1 (the expansion phase) HSPCs were transduced with a lentivirus expressing a mesothelin targeted chimeric antigen receptor (CAR). Expression of this CAR was driven using the EF1alpha promoter. This construct also contained a truncated CD19 (tCD19) extracellular domain, expressed from the same promoter and separated from the CAR sequence by a T2A self-cleaving domain. 1×10⁶ cells/mL in 0.5 mL of culture medium were plated in a 24-well plate that was precoated with a solution of recombinant human fibronectin fragment (Takara Bio, Inc.; RetroNectin® Recombinant Human Fibronectin Fragment) 5 μg/mL and 0.8 μg/cm² surface area), and a Notch agonist (Delta1^{ext-IgG}, 2.5 g/mL and 0.4 μg/cm² surface area) in DPBS (Gibco). Culture medium comprised a medium suitable for the growth and expansion of hematopoietic cells (StemSpan™ Serum Free Expansion Medium II (SFEM II; StemCell Technologies)) supplemented with 50 ng/mL each recombinant human SCF (Miltenyi Biotec), Flt-3 ligand (Miltenyi Biotec), TPO (Miltenyi Biotec), and IL-6 (Miltenyi Biotec), and 10 ng/ml IL-3 (Miltenyi Biotec). 8 μg/mL protamine sulfate (Millipore Sigma) was added along with the solution of lentivirus using a multiplicity of infection (MOI) of 30. A second addition of lentivirus at MOI of 30 was added to the well 3 hours later. Cells were incubated overnight at 37° C. in a 5% CO₂ incubator. On day 8, cells were passaged into a larger volume vessel (6-well plate) pre-coated with RetroNectin® and Delta1^{ext-IgG} by diluting cells in SFEM II media plus the above 5 growth factors to maintain a cell density <2×10⁶ cells/mL. On day 14, the cells were collected and replated at a density of 1×10⁶ cells/mL into tissue culture vessels (T25 tissue culture flasks) without the Delta1^{ext-IgG} or Retronectin® coatings. Differentiation culture medium comprised RPMI 1640 (Gibco) supplemented with 5% human platelet lysate (hPL) (Mill Creek Life Sciences), 40 ng/mL rhIL-15 (PeproTech), and 50 U/mL rhIL-2 (PeproTech). Cells were cultured for a total of 14 additional days, replacing 50% of the medium in the vessel with fresh differentiation medium on Day 21.

On day 28, the CAR NK cell preparation was collected and evaluated for CAR expression by flow cytometry and in an in vitro cytotoxicity assay against two different green fluorescent protein− (GFP−) expressing NOMO-1 AML tumor target cells. The parental NOMO-1 cell line endogenously expresses mesothelin that the CAR is targeted against. A knockout of mesothelin was prepared in this same cell line (NOMO-1^{MSLN-/-}).

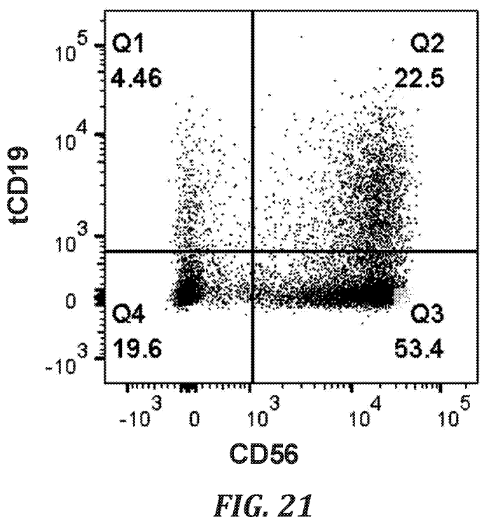

CAR expression was evaluated by indirect detection on the cell surface using an anti-CD19 antibody by flow cytometry. Cells were 76% CD56⁺ and 27% tCD19⁺ (FIG. 21).

CAR transduced and mock transduced control NK cells were tested as effector cells in an in vitro cytotoxicity assay by co-culturing with each of the two NOMO-1 target cells (parental and mesothelin knockout) in RPMI+10% FBS+40 ng/ml IL-15+50 U/mL IL-2. E:T ratios ranged between 0.3 to 20 and the assay proceeded for 24 hr. At the end of the incubation cells were labeled with a DAPI solution and analyzed by flow cytometry for GFP fluorescence of target cells and DAPI fluorescence of dead cells to determine the % specific cell death.

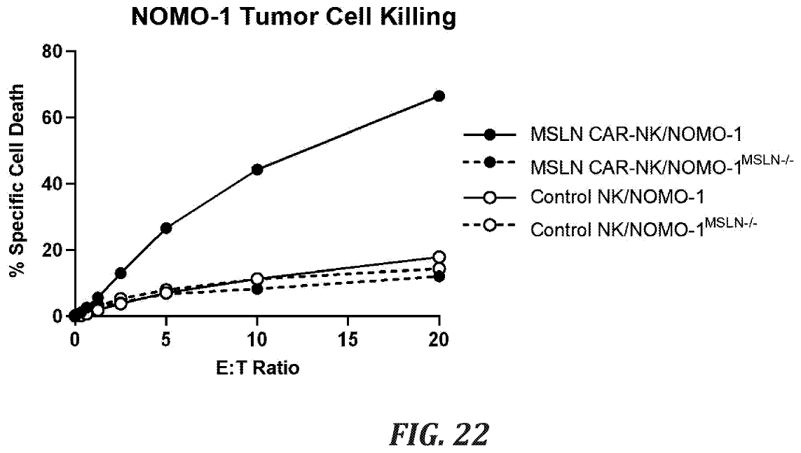

Results showed that the mesothelin CAR-NK cells demonstrated a significant and specific increase (up to 55%) in killing the parental NOMO-1 cells compared to the NOMO-1^{MSLN-/-} cells. Similar low-level killing was observed with the control NK cells against either NOMO-1 cell line (FIG. 22).

### Example 14: Generation of Mesothelin CAR NK Cells-Transduction after NK Cell Differentiation

In this example a CD34⁺ enriched non-immunologically matched pool of cells were processed as above through both phase 1 (expansion) and phase 2 (differentiation). Subsequent to full differentiation the NK cells were transduced with a CAR construct.

In particular, on day 23 of Phase 2 during the differentiation phase, bulk cells were enriched for human CD56⁺ NK cells using an immunomagnetic microbead based column isolation protocol (CD56 MicroBeads, human, Miltenyi Biotec). Enriched NK cells (>96% CD56⁺) were transduced with same lentivirus described in Example 12. 1×10⁶ cells/mL in 2 mL differentiation culture medium were plated in a 6-well plate along with 8 μg/mL protamine sulfate and lentivirus using an MOI of 40. Cells were incubated overnight at 37° C. in a 5% CO₂ incubator. On day 24, half the media volume was replaced with fresh culture medium.

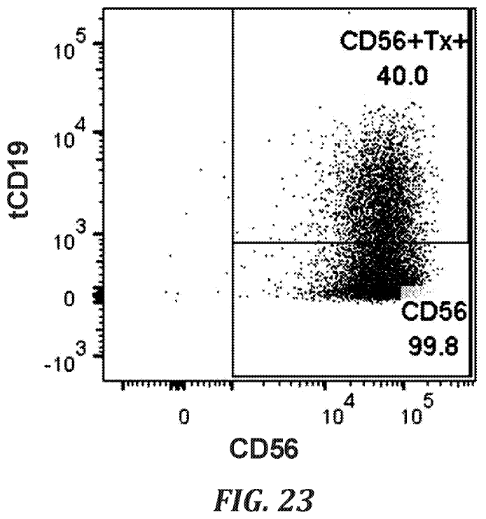

On day 27, NK cell preparations were collected and evaluated for CAR expression by flow cytometry and in an in vitro cytotoxicity assay against the parental NOMO-1 and NOMO-1^{MSLN-/-} target cells described in Example 13. CAR expression was evaluated by indirect detection on the cell surface using an anti-CD19 antibody by flow cytometry. Cells were >99% CD56⁺ and 40% tCD19⁺ (FIG. 23).

CAR transduced and mock transduced control NK cells were tested as effector cells in an in vitro cytotoxicity assay by co-culturing with each of the two NOMO-1 target cells (parental and mesothelin knockout) in RPMI+10% FBS+40 ng/ml IL-15+50 U/mL IL-2. E:T ratios ranged between 0.3 to 10 and the assay proceeded for 24 hr. At the end of the incubation cells were labeled with a DAPI and analyzed by flow cytometry for GFP fluorescence of target cells and DAPI fluorescence of dead cells to determine the % specific cell death.

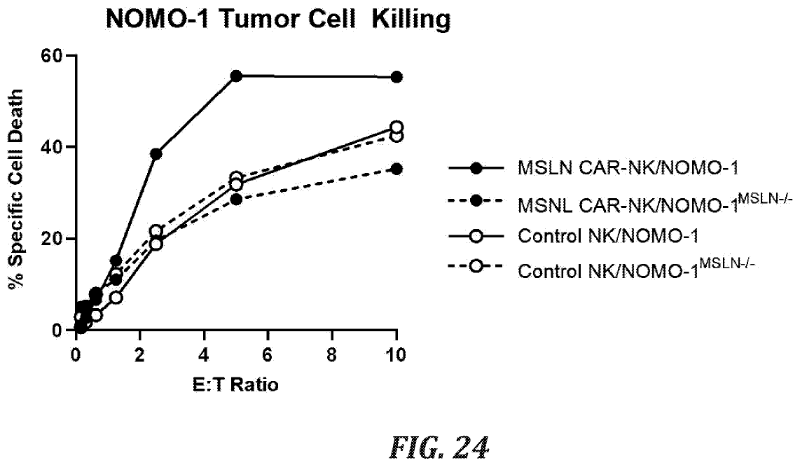

Results showed that the mesothelin CAR-NK cells demonstrated a significant and specific increase (up to 27%) in killing the parental NOMO-1 target cells compared to the NOMO-1^{MSLN-/-} target cells. Similar low-level killing was observed with the control NK cells against either NOMO-1 target cell line (FIG. 24).

The embodiments of the invention in which an exclusive property or privilege is claimed are defined as follows:

1. A method of preparing a genetically modified natural killer (NK) cell composition or preparation, consisting of:
selecting a plurality of umbilical cord blood or placental blood cells without immunological matching to each other;
preparing enriched cluster of differentiation 34 positive (CD34⁺) hematopoietic stem and progenitor cells (HSPCs) that are depleted of red blood cells and T cells;
culturing the CD34⁺ enriched HSPCs in an expansion culture medium comprising interleukin-3 (IL-3), interleukin-6 (IL-6), thrombopoietin (TPO), Fms-related tyrosine kinase 3 ligand (Flt-3 ligand (Flt-3L)), and stem cell factor (SCF) on a solid phase coated with a Notch ligand and recombinant human fibronectin or fragments thereof, and in the absence of exogenous feeder cells, for at least 7 days to produce expanded HSPCs, wherein the expanded HSPCs do not differentiate to produce more cluster of differentiation 56 positive (CD56⁺) cells than CD34⁺ cells during the expansion; and
culturing the expanded HSPCs in a differentiation culture medium comprising effective amounts of interleukin-2 (IL-2) and interleukin-15 (IL-15), a non-animal sourced serum replacement, and in the absence of the exogenous feeder cells for at least 7 days to produce an NK cell composition or preparation comprising 50 to 80% CD56⁺ cells and 20 to 50% endogenous CD56 negative (CD56⁻) cells, or 50 to 80% CD56⁺ cells and 15 to 50% endogenous CD56⁻ cells;
wherein the CD56⁺ cells express a high frequency of 60 to 100% of natural cytotoxicity receptor 3 (NKp30), natural cytotoxicity receptor 1 (NKp46), natural cytotoxicity receptor 2 (NKp44), immune inhibitory receptor natural killer group 2 member A (NKG2A), and granzyme B, a moderate to high frequency of 20 to 100% of perforin and lysosomal-associated membrane protein 1 (LAMP-1) or cluster of differentiation 107a (CD107a), a low to moderate frequency of 1 to 60% of cluster of differentiation 16 (CD16), and substantially no expression or less than 1% of killer-cell immunoglobulin-like receptors (KIRs); and wherein the CD56⁻ cells express a moderate to high frequency of 20 to 100% of granzyme B, a high frequency of 60 to 100% of CD107a, and a low frequency of 1 to 20% of perforin;
wherein the cells of the NK cell composition or preparation are genetically modified.

2. The method of claim 1, wherein genetic modification is during the expansion phase or subsequent to differentiation of the NK cells.

3. The method of claim 1, wherein the cells of the NK cell composition or preparation are genetically modified to express an antigen recognizing receptor.

4. The method of claim 3, wherein the genetic modification is introduction of a polynucleotide expressing a TCR of a CAR.

5. The method of claim 4, wherein the TCR or the CAR specifically binds to a viral antigen, a bacterial antigen, a tumor specific, or tumor associated antigen.

6. The method of claim 5, wherein the viral antigen is present in a Cytomegalovirus (CMV), an Epstein Barr Virus (EBV), a Human Immunodeficiency Virus (HIV), a Herpes simplex virus (HSV), a Hepatitis virus, a zika virus, an influenza virus, or a coronavirus.

7. The method of claim 6, wherein the Herpes virus is HSV 1 or HSV 2, the Hepatitis virus is Hepatitis A, B, or C, and the coronavirus is SARS-CoV or SARS-CoV-2.

8. The method of claim 5, wherein the tumor specific or tumor associated antigen is carbonic anhydrase IX (CAIX), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell, cutaneous lymphocyte-associated antigen (CLA), epithelial glycoprotein-2 (EGP-2), epithelial glycoprotein-40 (EGP-40), epithelial cell adhesion molecule (EpCAM), receptor tyrosine protein kinases erb-B2, erb-B3, erb-B4, folate-binding protein (FBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13Rα2), kappa light chain, kinase insert domain receptor (KDR), Lewis Y (LeY), L1 cell adhesion molecule (L1CAM), melanoma antigen family A, 1 MAGE-A1, mucin 16 (MUC16), mucin 1 (MUC1), mesothelin (MSLN), ERBB2, MAGEA3, p53, MARTI, gp100, Proteinase 3 (PR1), tyrosinase, Survivin, hTERT, EphA2, an NKG2D ligand, cancer-testis antigen NY-ESO-1, oncofetal antigen (h5T4), prostate stem cell antigen (PSCA), prostate specific membrane antigen (PSMA), RORI, tetraspanin 8 (TSPAN8), tumor-associated glycoprotein 72 (TAG-72), vascular endothelial growth factor R2 (VEGF-R2), Wilms tumor protein (WT-1), BCMA, GPC3, NKCS1, EGF1R, EGFR-VIII, CRLF2, or ERBB.

9. A genetically modified natural killer (NK) cell composition or preparation for use in immunotherapy, the NK cell composition or preparation comprising a population of NK cells of: 50 to 80% CD56⁺ cells and 20 to 50% CD56⁻ cells, or 50 to 85% CD56⁺ cells and 15 to 50% CD56⁻ cells, wherein the CD56⁺ cells express a high frequency of 60 to 100% of NKp30, NKp46, NKp44, NKG2A, NKG2D, and granzyme B, a moderate to high frequency of 20 to 100% of perforin and CD107a, a low to moderate frequency of 1 to 60% of CD16 and substantially no expression or less than 1% of KIRs; and wherein the CD56⁻ cells express a moderate to high frequency of 60 to 100% of granzyme B, a high frequency of 60 to 100% of CD107a, and a low frequency of 1 to 20% of perforin, and a pharmaceutically acceptable carrier.

10. The NK cell composition or preparation according to claim 9, wherein the cells of the composition or preparation are genetically modified to express an antigen recognizing receptor.

11. The NK cell composition or preparation according to claim 10, wherein the genetic modification comprises introduction of a polynucleotide expressing a TCR or a CAR.

12. The NK cell composition or preparation according to claim 11, wherein the TCR or the CAR specifically binds to a viral antigen, a bacterial antigen, or a tumor associated or tumor specific antigen.

13. The NK cell composition or preparation according to claim 12, wherein the viral antigen is present in a Cytomegalovirus (CMV), an Epstein Barr Virus (EBV), a Human Immunodeficiency Virus (HIV), a Herpes simplex virus (HSV), a Hepatitis virus, a zika virus, an influenza virus, or a coronavirus.

14. The NK cell composition or preparation according to claim 13, wherein the Herpes virus is HSV 1 or HSV 2, the Hepatitis virus is Hepatitis A, B, or C, and the coronavirus is SARS-CoV or SARS-CoV-2.

15. The NK cell composition or preparation according to claim 12, wherein the tumor specific or tumor associated antigen is carbonic anhydrase IX (CAIX), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell, cutaneous lymphocyte-associated antigen (CLA), epithelial glycoprotein-2 (EGP-2), epithelial glycoprotein-40 (EGP-40), epithelial cell adhesion molecule (EpCAM), receptor tyrosine protein kinases erb-B2, erb-B3, or erb-B4, folate-binding protein (FBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13Rα2), kappa light chain, kinase insert domain receptor (KDR), Lewis Y (LeY), L1 cell adhesion molecule (L1CAM), melanoma antigen family A, 1 (MAGE-AI), mucin 16 (MUC16), mucin 1 (MUC1), mesothelin (MSLN), ERBB2, MAGEA3, p53, MARTI, gp100, proteinase3 (PR1), tyrosinase, survivin, hTERT, ephA2, an NKG2D ligand, cancer-testis antigen NY-ESO-1, oncofetal antigen (h5T4), prostate stem cell antigen (PSCA), prostate specific membrane antigen (PSMA), ROR1, tetraspanin 8 (TSPAN8), tumor-associated glycoprotein 72 (TAG-72), vascular endothelial growth factor R2 (VEGF-R2), Wilms tumor protein (WT-1), BCMA, GPC3, NKCS1, EGF1R, EGFR-VIII, CRLF2, or ERBB.

16. The NK cell composition or preparation of claim 15, wherein the tumor specific or tumor associated antigen is CD19, ROR1, HER2, PSMA, PSCA, mesothelin, or CD20.

17. The NK cell composition or preparation of claim 11, wherein the CAR further comprises an intracellular signaling domain comprising a signaling domain of CD3zeta, CD28, and 4-1BB; at least one co-stimulatory domain comprising a co-stimulatory domain of CD27, CD28, 4-1BB, 2B4, DAP10, DAP12, OX40, CD30, CD40, lymphocyte function associated antigen-1 (LFA-1), CD2, CD7, LIGHT, NKG2C, or B7-H3; a transmembrane domain comprising a transmembrane domain of CD8, CD28, CD3zeta, CD4, 4-1BB, OX40, ICOS, or NKG2D; and a spacer region comprising a hinge region of IgG₁, the CH2CH3 region of an immunoglobulin, a portion of CD3, a portion of CD28, or a portion of CD8.

18. The NK cell composition or preparation of claim 1, wherein the NK cell composition comprises from 50 million to 2 billion viable NK cells.

19. A method for treating a subject in need thereof, comprising administering a therapeutically effective amount of the NK cell composition or preparation of claim 1 to the subject.

20. The method of claim 19, wherein the subject has a viral or a bacterial infection, or has a cancer expressing a tumor antigen and the genetically modified NK cells of the composition or preparation express an antigen recognizing receptor that binds to the virus, or bacteria, or binds to the tumor antigen.


SEQUENCE LISTING (75 sequences)

SEQ ID NO: 1 - protein, 11 aa, synthetic construct
RASQDISKYLN

SEQ ID NO: 2 - protein, 7 aa, synthetic construct
SRLHSGV

SEQ ID NO: 3 - protein, 9 aa, synthetic construct
GNTLPYTFG

SEQ ID NO: 4 - protein, 5 aa, synthetic construct
DYGVS

SEQ ID NO: 5 - protein, 16 aa, synthetic construct
VTWGSETTYYNSALKS

SEQ ID NO: 6 - protein, 7 aa, synthetic construct
YAMDYWG

SEQ ID NO: 7 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 8 - protein, 247 aa, Homo sapiens
MASPLTRELSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 9 - protein, 69 aa, synthetic construct
STTTKPVLRTPSPVHPTGTSQPQRPEDCRPRGSVKGTGLDFACDIYIWAPLAGICVALLLSLIITLICY

SEQ ID NO: 10 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 188 aa, synthetic construct
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 13 - protein, 113 aa, synthetic construct
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 14 - protein, 218 aa, synthetic construct
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 15 - DNA, 767 bp, Homo sapiens
  source 1..767: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 16 - DNA, 601 bp, Homo sapiens
  source 1..601: mol_type = other DNA
atgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaaggggggcaaatactctgcgatctcactaagacaaaaggaagtggaaacacagtgtccattaagagtctgaaattctgccattctcagttatccaacaacagtgtctctttttttctacaaccttggaccattctcatgccaactattacttctgcaacctatcaatttttgatcctcctccttttaaagtaactcttacaggaggatatttgcatatttatgaaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 17 - DNA, 282 bp, Homo sapiens
  source 1..282: mol_type = other DNA
atgatccatctgggtcacatcctcttcctgcttttgctcccagtggctgcagctcagacgaccccaggagagagatcatcactccctgccttttaccctggcacttcaggctcctgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctga

SEQ ID NO: 18 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 19 - protein, 277 aa, synthetic construct
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 20 - protein, 199 aa, synthetic construct
MKSGLWYFFLFCLRIKVLTGEINGSANYEMFIFHNGGVQILCKYPDIVQQFKMQLLKGGQILCDLIKTKGSGNTVSIKSLKFCHSQLSNNSVSFFLYNLDHSHANYYFCNLSIFDPPPFKVTLIGGYLHIYESQLCCQLKFWLPIGCAAFVVVCILGCILICWLTKKKYSSSVHDPNGEYMFMRAVNTAKKSRLTDVTL

SEQ ID NO: 21 - protein, 485 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 462 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 23 - protein, 491 aa, synthetic construct
MALPVTALLLPLALLLHAEVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSSGGGGSGGGGSGGGGSDIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 491 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 121 aa, synthetic construct
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 26 - protein, 115 aa, synthetic construct
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRAD

SEQ ID NO: 27 - protein, 116 aa, synthetic construct
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDP

SEQ ID NO: 28 - protein, 15 aa, synthetic construct
GGGGSGGGGSGGGGS

SEQ ID NO: 29 - protein, 117 aa, synthetic construct
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 30 - protein, 112 aa, synthetic construct
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 31 - protein, 119 aa, synthetic construct
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 32 - protein, 106 aa, synthetic construct
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 33 - protein, 120 aa, synthetic construct
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTV

SEQ ID NO: 34 - protein, 107 aa, synthetic construct
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 35 - protein, 117 aa, synthetic construct
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 36 - protein, 106 aa, synthetic construct
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 37 - protein, 118 aa, synthetic construct
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 38 - protein, 107 aa, synthetic construct
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 39 - protein, 120 aa, synthetic construct
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 40 - protein, 107 aa, synthetic construct
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 41 - protein, 119 aa, synthetic construct
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 42 - protein, 107 aa, synthetic construct
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 43 - protein, 123 aa, synthetic construct
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 44 - protein, 107 aa, synthetic construct
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 45 - protein, 244 aa, synthetic construct
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSSGGGGSGGGGSGGGGSQIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 46 - protein, 244 aa, synthetic construct
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIKGGGGSGGGGSGGGGSEVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 47 - protein, 240 aa, synthetic construct
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSSGGGGSGGGGSGGGGSQIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 48 - protein, 240 aa, synthetic construct
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELKGGGGSGGGGSGGGGSQVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 49 - protein, 244 aa, synthetic construct
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSAGGGGSGGGGSGGGGSDIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 50 - protein, 244 aa, synthetic construct
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELKGGGGSGGGGSGGGGSEVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSA

SEQ ID NO: 51 - protein, 238 aa, synthetic construct
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSSGGGGSGGGGSGGGGSENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 52 - protein, 238 aa, synthetic construct
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIKGGGGSGGGGSGGGGSEVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 53 - protein, 240 aa, synthetic construct
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSSGGGGSGGGGSGGGGSDIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 54 - protein, 240 aa, synthetic construct
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELKGGGGSGGGGSGGGGSQVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 55 - protein, 242 aa, synthetic construct
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSSGGGGSGGGGSGGGGSDIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 56 - protein, 242 aa, synthetic construct
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELKGGGGSGGGGSGGGGSQVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 57 - protein, 241 aa, synthetic construct
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSSGGGGSGGGGSGGGGSDIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 58 - protein, 241 aa, synthetic construct
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIKGGGGSGGGGSGGGGSEVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 59 - protein, 245 aa, synthetic construct
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSSGGGGSGGGGSGGGGSDIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 60 - protein, 245 aa, synthetic construct
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELKGGGGSGGGGSGGGGSEVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 61 - protein, 842 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 62 - protein, 647 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 6 aa, synthetic construct
DNSKSQ

SEQ ID NO: 64 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 66 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 67 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 68 - protein, 161 aa, synthetic construct
RISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - DNA, 1944 bp, synthetic construct
  source 1..1944: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 70 - protein, 725 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 71 - DNA, 2177 bp, synthetic construct
  source 1..2177: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgtggaggaggaagaggaaggagaagcagagcgagacaagccctaaggagtttctgacaatctatgaagacgtgaaggacctgaagacacggagaaaccacgagcaggagcagaccttccctggaggaggcagcacaatctactccatgatccagtctcagagcagcgcccccacctcccaggagcctgcctacacactgtatagcctgatccagccatcccggaagtctggcagcaggaagcgcaaccactccccctcttttaattctaccatctatgaagtgatcggcaagagccagcccaaggcacagaaccccgcacgactgagcaggaaggaactggagaactttgatgtctactctagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggagagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 72 - protein, 640 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPPFFFCCFIAVAMGIRFIIMVTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - DNA, 1923 bp, synthetic construct
  source 1..1923: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctcccttctttttctgctgttttatcgccgtggctatgggcatccggttcatcatcatggtgaccaaacggggcagaaagaaactcctgtatatattcaaacaaccatttatgagaccagtacaaactactcaagaggaagatggctgtagctgccgatttccagaagaagaagaaggaggatgtgaactgagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 74 - protein, 718 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - DNA, 2157 bp, synthetic construct
  source 1..2157: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctcccttctttttctgctgttttatcgccgtggctatgggcatccggttcatcatcatggtgacctggaggaggaagaggaaggagaagcagagcgagacaagccctaaggagtttctgacaatctatgaagacgtgaaggacctgaagacacggagaaaccacgagcaggagcagaccttccctggaggaggcagcacaatctactccatgatccagtctcagagcagcgcccccacctcccaggagcctgcctacacactgtatagcctgatccagccatcccggaagtctggcagcaggaagcgcaaccactccccctcttttaattctaccatctatgaagtgatcggcaagagccagcccaaggcacagaaccccgcacgactgagcaggaaggaactggagaactttgatgtctactctagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa